(19) United States
(12) Patent Application Publication
(10) Pub. No.: US 2024/0158784 A1
(43) Pub. Date: May 16, 2024

(54) MOLECULAR BARCODES AND RELATED METHODS AND SYSTEMS

(71) Applicant: Oregon Health & Science University, Portland, OR (US)

(72) Inventor: Corey M. Dambacher, Portland, OR (US)

(73) Assignee: Oregon Health & Science University, Portland, OR (US)

(21) Appl. No.: 18/548,864

(22) PCT Filed: Mar. 4, 2022
(86) PCT No.: PCT/US22/19045

§ 371 Date: Sep. 1, 2023

Related U.S. Application Data
(60) Provisional application No. 63/156,858, filed on Mar. 4, 2021.

(51) Int. Cl. C12N 15/10

(52) U.S. Cl. CPC C12N 15/1065

(57) ABSTRACT

The present disclosure relates to molecular barcodes comprising a set of one or more identimers. Methods of using the molecular barcodes, such as to classify a material, are disclosed. Methods of making the molecular barcodes and systems for using the molecular barcodes are disclosed herein.

### CROSS-REFERENCE TO RELATED APPLICATIONS

This is the 371 National Phase of International Application No. PCT/US22/19045, filed on Mar. 4, 2022, which claims priority to and the benefit of the earlier filing of U.S. Provisional Application No. 63/156,858, filed on Mar. 4, 2021, each of which is incorporated by reference herein in its entirety.

### REFERENCE TO SEQUENCE LISTING

The nucleic acid and/or amino acid sequences described herein are shown using standard letter abbreviations, as defined in 37 C.F.R. § 1.822. Only one strand of each nucleic acid sequence is shown, but the complementary strand is understood as included in embodiments where it would be appropriate. A computer readable text file, entitled “0046-0083US_SeqList.txt” created on or about Sep. 1, 2023, with a file size of 20,000 bytes, contains the sequence listing for this application and is hereby incorporated by reference in its entirety.

### COPYRIGHT NOTICE

© 2023 Oregon Health & Science University. A portion of the disclosure of this patent document contains material that is subject to copyright protection. The copyright owner has no objection to the facsimile reproduction by anyone of the patent document or the patent disclosure, as it appears in the Patent and Trademark Office patent file or records, but otherwise reserves all copyright rights whatsoever. 37 CFR § 1.71(d).

### TECHNICAL FIELD

This disclosure relates to biotechnology. More specifically, this disclosure relates to molecular barcodes and related methods and systems

### BRIEF DESCRIPTION OF THE DRAWINGS

FIG. 1 is a graphical representation of construction steps for non-DNA barcodes.

FIG. 2 is a graphical representation of construction steps for barcodes linked to Next Generation Sequencing (NGS) barcodes.

FIG. 3 is a graphical representation of a non-nucleic acid barcode and potential imaging results during deconvolution of the barcode.

FIG. 4 is a graphical representation of construction steps for unlabeled barcodes linked to NGS barcodes. These may be optionally labeled.

FIG. 5 is a graphical representation of construction steps to encode chemical libraries without using DNA.

FIG. 6 is a graphical representation of construction steps for cleavable barcodes linked to NGS barcodes.

FIG. 7 is a graphical representation of decoding steps to identify non-DNA molecular barcodes.

FIG. 8 is a graphical representation of decoding steps to identify barcodes linked to NGS barcodes.

FIG. 9 is a graphical representation of a double stranded DNA NGS-compatible barcodes with endonuclease recognition sites and potential imaging results during deconvolution of the barcode.

FIG. 10 is a graphical representation of a non-nucleic acid NGS-compatible barcode with orthogonal protease recognition sites and potential imaging results during deconvolution of the barcode.

FIG. 11 is a graphical representation of deconvolution steps of a visual NGS barcode.

FIG. 12 is a graphical representation of construction of a non-DNA barcode linked to chemical building blocks.

FIG. 13 is a graphical representation of a non-DNA barcode encoded chemical library and potential imaging results during deconvolution of the barcode.

FIG. 14 is a graphical representation of an exemplary dual labeled, double stranded DNA identimer.

FIG. 15 is a graphical representation of a scaffolding portion connected to detectable labels.

FIG. 16 is a graphical representation of a non-nucleic acid barcode with dual labeled cyclic peptide building blocks and how the barcode may be decoded.

FIG. 17 is a graphical representation of a mini well with capping beads that may be a surface for attaching barcodes for single cell analysis.

FIG. 18 is a textual representation of a set of five single-label FND identimers having scaffold portions configured with orthogonal sticky-ends, orthogonal recognition moieties and cleavage sites as well as modified nucleotides.

FIG. 19A represents a streptavidin bead labeled with a first labeled dsDNA identimer segment (Id₁/HindIII-AF750) attached through ligation to an unlabeled dsDNA that is attached to the bead through a biotin moiety.

FIG. 19B represents a streptavidin bead labeled with a second labeled dsDNA identimer segment (Id₂/SpeI-AF647).

FIG. 19C represents a streptavidin bead labeled with a third labeled dsDNA identimer segment (Id₃/XhoI-ATT0550).

FIG. 19D represents a streptavidin bead labeled with a fourth labeled dsDNA identimer segment (Id₄/NotI-ATT0488).

FIG. 20A represents un-cleaved beads imaged in four fluorescence channels; 647 nm (upward diagonal stripes), 550 nm (downward diagonal stripes), 488 nm (horizontal stripes) and 750 nm (dotted), and mean intensity values obtained for each fluorophore were plotted to the right.

FIG. 20B represents mean intensity of beads following exposure to the NotI enzyme.

FIG. 20C represents mean intensity of beads following exposure to the XhoI enzyme.

FIG. 20D represents mean intensity of beads following exposure to the SpeI enzyme.

FIG. 21A presents a bar graph representing OCS results obtained for single-label dsDNA identimer chains where the order of the cycles was reversed for analysis.

FIG. 21B presents a bar graph representing OCS results obtained for mixed-label dsDNA identimer chains where the order of the cycles was reversed for analysis.

FIG. 22A represents the first of six I beads bearing the same single-label identimer chain sequence used to generate data from individual beads in a FOV tracked over 3 cycles of an OCS experiment.

FIG. 22B represents the second of six I beads bearing the same single-label identimer chain sequence used to generate data from individual beads in a FOV tracked over 3 cycles of an OCS experiment.

FIG. 22C represents the third bead bearing the same single-label identimer chain sequence used to generate data from individual beads in a FOV tracked over 3 cycles of an OCS experiment.

FIG. 22D represents the fourth bead bearing the same single-label identimer chain sequence used to generate data from individual beads in a FOV tracked over 3 cycles of an OCS experiment.

FIG. 22E represents the fifth bead bearing the same single-label identimer chain sequence used to generate data from individual beads in a FOV tracked over 3 cycles of an OCS experiment.

FIG. 22F represents the sixth bead bearing the same single-label identimer chain sequence used to generate data from individual beads in a FOV tracked over 3 cycles of an OCS experiment.

FIG. 23A graphs the bead library for a bead labeled with the sequence Id₁/HindIII-AF647-Id₂/SpeI-AF750-Id₃/XhoI-ATT0488-Id₄/NotI-ATT0488 across the OCS experiment.

FIG. 23B graphs the bead library for a bead labeled with the sequence Id₁/HindIII-ATT0488-Id₂/SpeI-ATT0550-Id₃/XhoI-AF647-Id₄/NotI-ATT0488 across the OCS experiment.

FIG. 23C graphs the bead library for a bead labeled with the sequence Id₁/HindIII-ATT0550-Id₂/SpeI-AF750-Id₃/XhoI-ATT0488-Id₄/NotI-AF750 across the OCS experiment.

FIG. 24A represents SpeI enzyme cleavage of an AF-750-labelled hairpin oligo containing two AF-750 labels.

FIG. 24B presents a graph of signals from both the AF-647 and AF-750 labels before and following SpeI enzyme cleavage.

FIG. 25A represents ligation of an ATTO-550 label from a streptavidin bead and confirmation by fluorescence imaging.

FIG. 25B represents a second labeled DNA hairpin, containing the AF-647 label orthogonally attached through ligation to the second of the three acceptor oligos on the bead and confirmation by fluorescence imaging.

FIG. 25C represents a third labeled DNA hairpin, containing the ATTO-488 label orthogonally attached through ligation to the last of three acceptor oligos on the bead and confirmation by fluorescence imaging.

FIG. 26A represents a bead with three different ring identimers, Id1/SpeI-ATTO-550, Id2/XhoI-AF-647, and Id3/NotI-ATTO-488 before and after SpeI enzyme cleavage.

FIG. 26B presents a bar graph demonstrating efficient SpeI cleavage of the ATTO-550 label.

FIG. 27A represents a first encoding cycle (ATTO-488 labeling) of a streptavidin bead containing a first labeled ssDNA that is hybridized to a first unlabeled ssDNA that is attached to the bead through a biotin moiety.

FIG. 27B represents a second encoding cycle (AF-647 labeling) of the streptavidin bead.

FIG. 28A represents a first cleavage with a first enzyme (NotI) on a bead containing three different hybridized identimers, Id₁/NotI-ATTO-488, Id₂/SpeI-AF-647, and Id₃/XhoI-ATTO-550.

FIG. 28B represents a second cleavage with a second enzyme (SpeI) on the bead containing three different hybridized identimers.

FIG. 28C provides a graph of fluorescence imaging confirmation of efficient NotI and SpeI enzymatic cleavage.

### DETAILED DESCRIPTION

In a first embodiment, the molecular barcodes described herein comprise a set of one or more identimers, where each identimer includes one or more detectable labels operatively connected to a scaffold portion. The scaffold portion comprises a recognition moiety that is orthogonal to the recognition moiety of at least one identimer in the set of identimers and a cleavage site operatively connected to the one or more detectable labels, concatenated to each other in a three-dimensional arrangement to encode and form the molecular barcode.

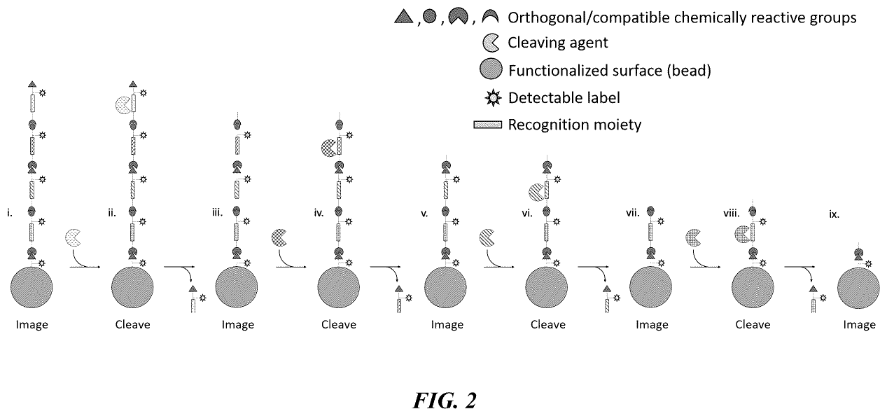

There may be instances where a molecular barcode includes an uncleavable identimer (FIG. 2). In some embodiments, an uncleavable identimer token may be included in a molecular barcode to facilitate detection of its signal response after the detectable labels of cleavable identimer tokens have dissociated from the molecular barcode. In some embodiments, an uncleavable identimer token may be operatively connected to a solid phase material as a firstly encoded identimer token. In some embodiments, uncleavable identimers comprise scaffolding portions lacking a chemically reactive recognition moiety or cleavage site.

The phrases “operatively coupled to” and “coupled to” refer to any form of interaction between two or more entities, including electrostatic, enzymatic, covalent, ionic, or other chemical interaction. Two entities may interact with each other even though they are not in direct contact with each other. For example, two entities may interact with each other through an intermediate entity.

As used herein, protease cleavage site sequences and protease recognition sequences are peptide sequences at which an orthogonally reactive protease binds. As used herein, a protease cleavage site sequences further includes a cleavage site within its peptide sequence.

The application of orthogonally reactive cleaving agents to the molecular barcode cleaves it at the cleavage sites of identimers having recognition moieties that are reactive to the cleaving agents to dissociate the detectable labels of the identimers from the three-dimensional arrangement according to the orthogonality of the identimers and thereby induce a detectable signal response to decode the molecular barcode.

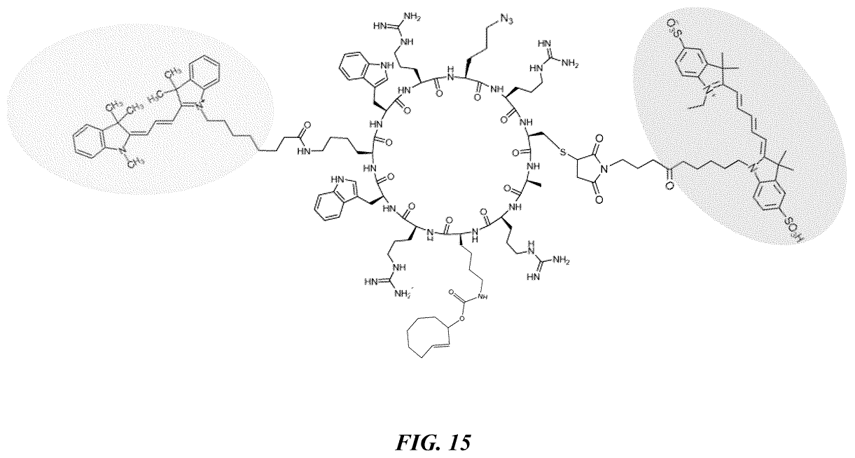

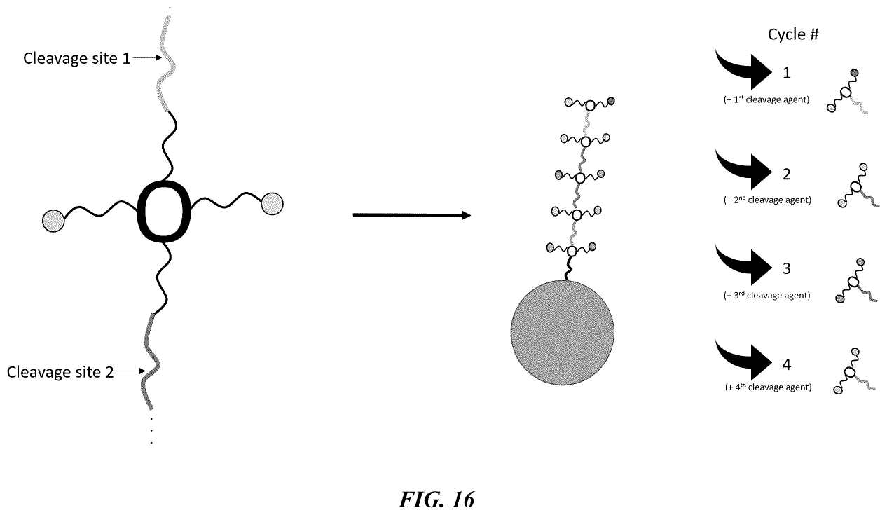

The scaffold portion of the molecular barcode may comprise polypeptides, amino acids, cyclic peptides, nucleic acids, or a combination thereof. The scaffold portion may comprise polyethylene glycol (PEG)n. “n” may equal 1-12, more preferably 1-6, most preferably 2-5. Examples of labeled cyclic peptides are shown in FIG. 15 and FIG. 16. Detectable labels may be fluorophores or chemically-protected quantum dots, for example. Spacing detectable labels away from each other can prevent quenching and/or spectral shifting. Cleavage sites may be a chemical linker, peptide, ssDNA, dsDNA, or RNA for cleavage by a small molecule, protease, endonuclease, or RNAse H respectively.

In some embodiments, the scaffold portion comprises N-terminal lysine residues, the N-terminal lysine residues being modifiable with a chemical group (e.g., transcyclooctene (TCO), using NHS-PEG₄-TCO) compatible for concatenating to the scaffold portions of other identimers containing a C-terminal methyltetrazine group. C-terminal modification of identimers may be achieved using commercially available heterobifunctional crosslinkers containing the maleimide reactive moiety for attachment to the installed cysteine sulfhydryl group (Methyltetrazine-PEG 4-Maleimide). In some embodiments, the scaffold portions may be modified through their C-terminal cysteine residues to contain the DBCO group (using DBCO-PEG₄-Maleimide) while the scaffold portions of N-terminal lysate-bearing identimers may be modified through the N-terminal lysine residues to contain the azide group. In some embodiments, a terminal identimer may be included in a molecular barcode to acts as a “capping” identimer, and therefore is not modified on its C-terminal end to contain a chemically reactive click moiety.

In some embodiments, an identimer scaffold portion comprises a peptide fragment covalently linked through its N- and C-terminal ends to linker reagents containing chemically reactive groups that enable the orthogonal chemical concatenation of identimer tokens using a split-pooling approach. In some embodiments, the recognition moiety of the identimer is a protease cleavage site sequences. In some embodiments, the recognition moiety of the identimer is a protease recognition sequence. In some embodiments, a identimer scaffold portion comprises a polypeptide fragment modified to contain an N-terminal lysine residue, a C-terminal cysteine residue, and an internal non-natural amino acid bearing the azide group, and may be ordered from a commercial supplier (e.g., Anaspec Inc., located at 34801 Campus Dr. Fremont, CA 94555).

In some embodiments, the scaffold portion comprises a N-terminal cysteine residue to facilitate concatenation of N-terminal cysteine bearing identimers to each other by native chemical ligation. In some embodiments, the scaffold portion comprises enzymatic recognition tags to facilitate concatenation of enzymatic recognition tag-bearing identimers.

In some embodiments, a click chemistry may be used to concatenate identimers because click reactions are orthogonal may have desirable reaction kinetics compared to native chemical ligation, possibly requiring less identimers to form molecular barcodes and thereby reduce costs.

In some embodiments, the scaffolding portion comprises a cyclic peptide, spacing linker, or other spacing molecule to facilitate concatenation of identimers through chemically linkage. Here, the cyclic peptide, spacing linker, or spacing molecule acts to space identimer monomeric units from each other by the molecular distance of the cyclic peptide, spacing linker, or spacing molecule.

In a preferred embodiment, a scaffolding portion is operatively connected to a set of one or more detectable labels. In embodiments comprising scaffolding portions operatively connected to two or more detectable labels (i.e., dual labeling,) the scaffolding portion may be configured to space the detectable labels of an identimer apart from one another. For example, spacing detectable labels apart relative to each other may enhance combinatorial labeling performance of identimers.

As shown in FIG. 16, a scaffolding portion comprises a cyclic peptide and may be compatibly attached (or inserted) between two identimers. In the configuration shown in FIG. 16, the orientation of each identimer would not be relevant (identimers can be concatenated by attachment of either their N- or C-terminal ends) because the detectable labels may be attached to the scaffolding portion between the identimers, and the orientation of the proteolytic site will not affect protease activity.

In some embodiments, a scaffold portions comprises a single-stranded DNA (ssDNA) molecule having a 5′ and 3′ end that is fluorescently labeled with a detectable label through a modified internal base, and is pre-hybridized with a complementary ssDNA to form a dsDNA duplex. The formed dsDNA duplex may encode one or more copies of a single endonuclease restriction sequence (or site type). One having ordinary skill in the art, with the benefit of this disclosure, will understand that efficient dissociation of detectable labels from a formed and encoded molecular barcode may be facilitated by selectively spacing the endonuclease restriction sequences along the scaffold portion. In some embodiments, one strand of the dsDNA duplex containing the nuclease recognition sequence(s) comprises an internal-amino modification for attachment of a pre-designated fluorophore.

In a preferred embodiment, the detectable labels comprise one or more Q-dots because of their brightness (i.e., quantum yield) and multiplexing capabilities. In some embodiments, the detectable labels comprise fluorophores having different emission wavelengths that may be covalently attached to the scaffold portion of an identimer. In some embodiments, the combination of detectable signal responses of a formed and encoded molecular barcode may or may not comprise contiguous emission spectra, as the three-dimensional arrangement of the identimer tokens forming the molecular barcode may vary.

In some embodiments, a molecular barcode may be formed and encoded to include about one million (10⁶) identimer class variations if Q-dots are used in parallel by split-pooling. Dual labeling of each identimer with combinatorial Q-dot labels may expand this number to about one billion (10¹⁰) identimer class variations. In some embodiments, the use of fluorophore-doped nanoparticles may provide further identimer class variations.

The recognition moiety of at least one identimer in the set of identimers may comprise a chemical linker, peptide, or nucleic acid. The cleavage site may be within the recognition moiety. In particular, each recognition moiety in the set of identimers may comprise at least one moiety selected from a protease recognition moiety, an endonuclease recognition moiety, an epitope recognizable by an affinity reagent, a nucleic acid probe recognition moiety, a modified peptide side chain, and an unnatural peptide side chain.

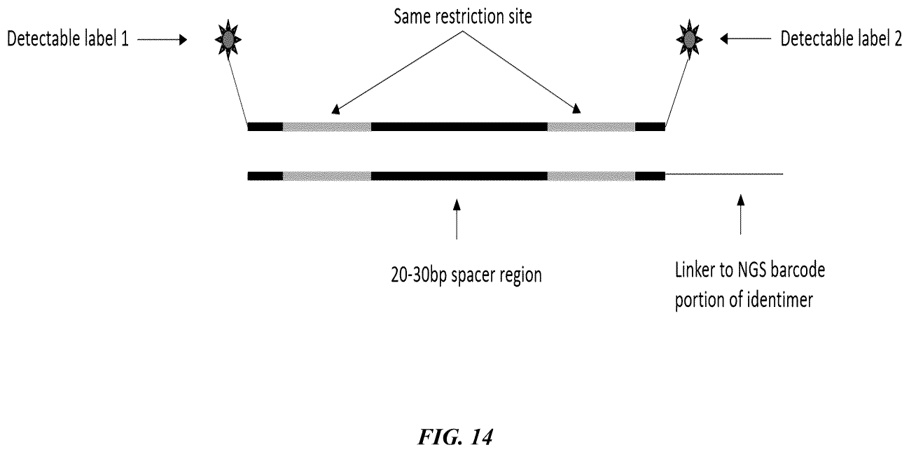

An exemplary identimer having two detectable labels and two of the same recognition site is shown in FIG. 14.

Molecular barcodes disclosed herein may be constructed by attaching an identimer to another identimer via specific compatible chemistries, such as click chemistry moieties, native chemical ligation linkers, or enzyme mediated ligation linkers, for example DNA ligation by T4 DNA ligase. The molecular barcode may further comprise an identimer linker between each identimer in the set of identimers. The identimer linker may be selected from a group of click chemistry moieties, native chemical ligation linkers, and enzyme-mediated ligation linkers.

In some embodiments, identimers may be sequentially added in a pre-defined order to form barcodes over sequential rounds of splitting and pooling. Each round of identimer addition will enable concatenation of identimers through orthogonal chemical ligation.

In some embodiments, concatenation of identimers by ligation may be performed in the same reaction buffer (1× Phosphate Buffered Saline (PBS) or 1×T4 DNA ligase buffer) using a molar excess of the incoming identimer to be concatenated.

In some embodiments, N-terminal lysine residues installed within scaffold portions may be modified with a chemical group (transcyclooctene (TCO), using NHS-PEG₄-TCO) compatible for attachment to other identimers that contain a C-terminal methyltetrazine group. C-terminal modification of identimers may be achieved using commercially available heterobifunctional crosslinkers containing the maleimide reactive moiety for attachment to the installed cysteine sulfhydryl group (Methyltetrazine-PEG₄-Maleimide). In some embodiments, some identimers may be modified through their C-terminal cysteine residues to contain the DBCO group (using DBCO-PEG 4-Maleimide), while some identimers may be modified through their N-terminal lysine residues to contain the azide group. In some embodiments, a terminal identimer is included in the formed molecular barcode and acts as a “capping” identimer, and therefore is not modified on its C-terminal end to contain a chemically reactive click moiety.

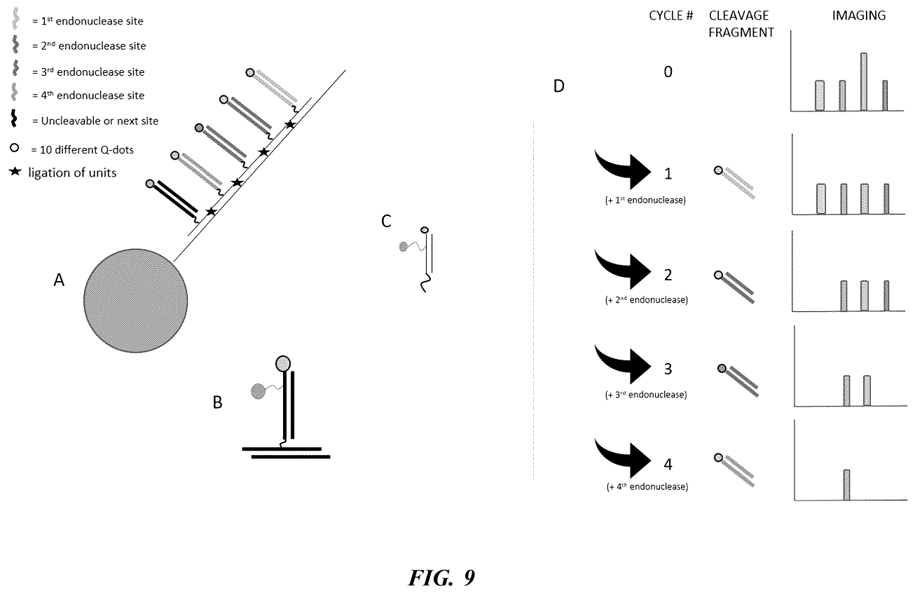

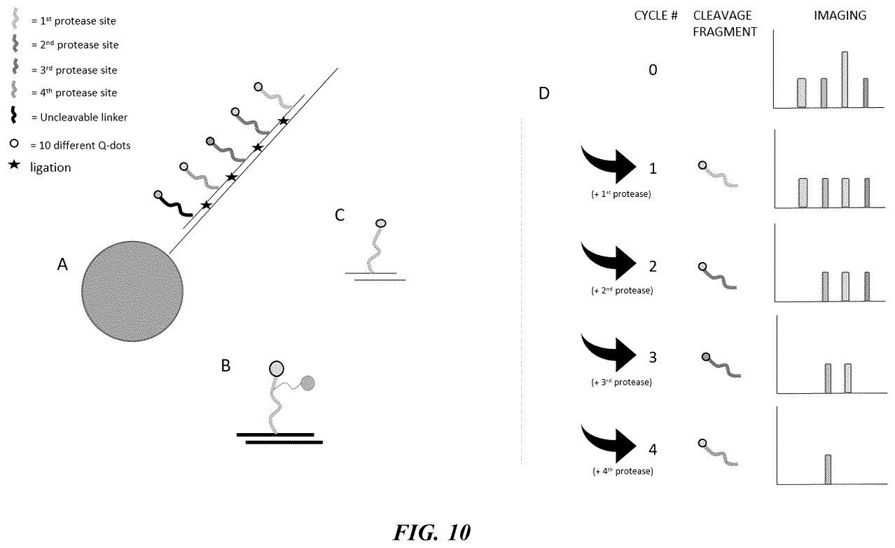

Molecular barcodes linked to Next Generation Sequencing barcodes may have the scaffolding portion comprising double stranded DNA and the recognition moiety comprising protein, DNA, RNA, or a chemically cleavable moiety. The double stranded DNA may have a 3′ overlap for capture such as Poly(T), a unique molecular identifier, and/or a 5′ PCR handle (FIG. 9 and FIG. 10).

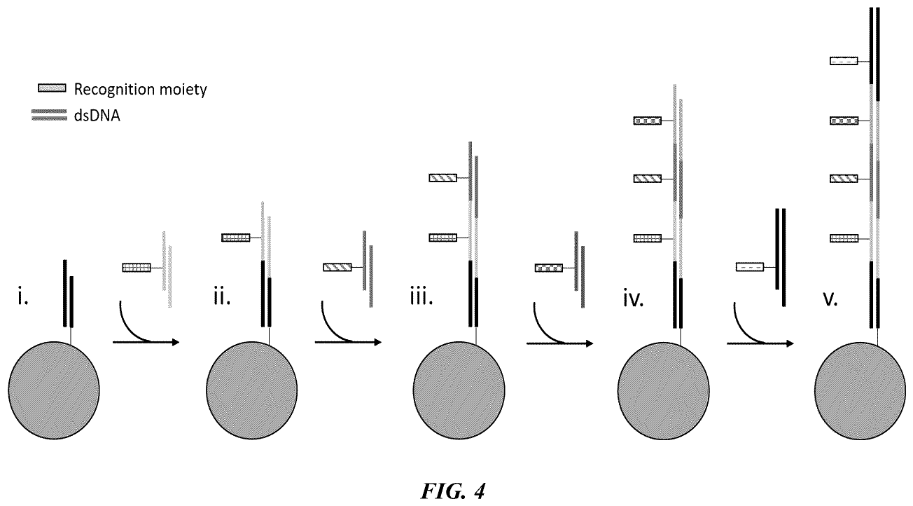

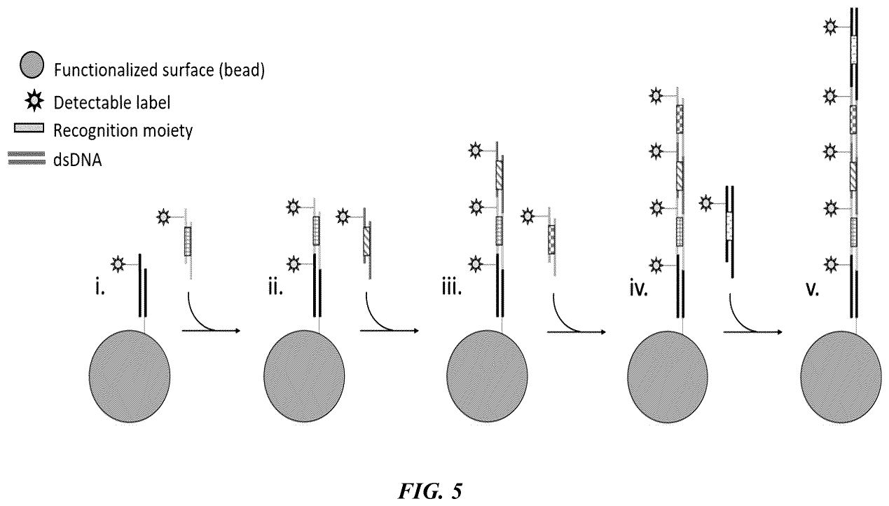

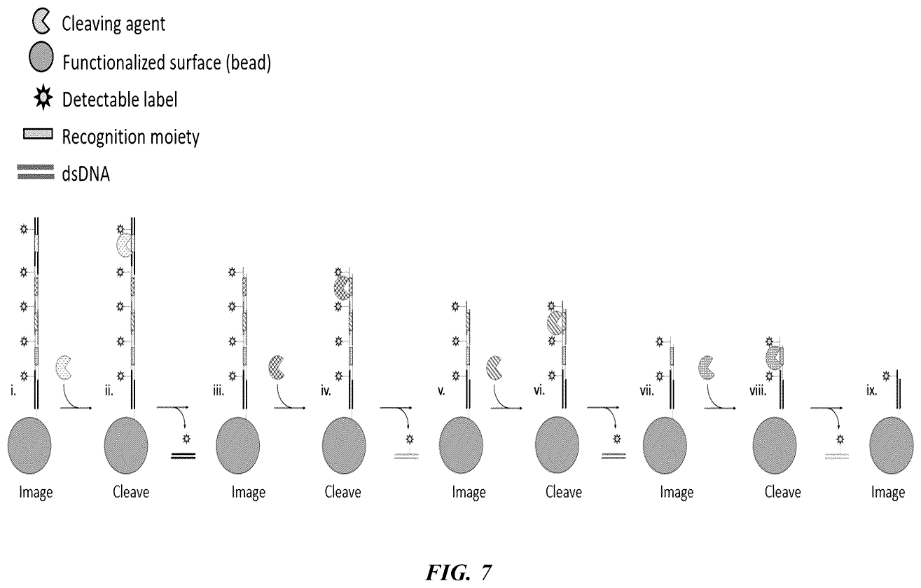

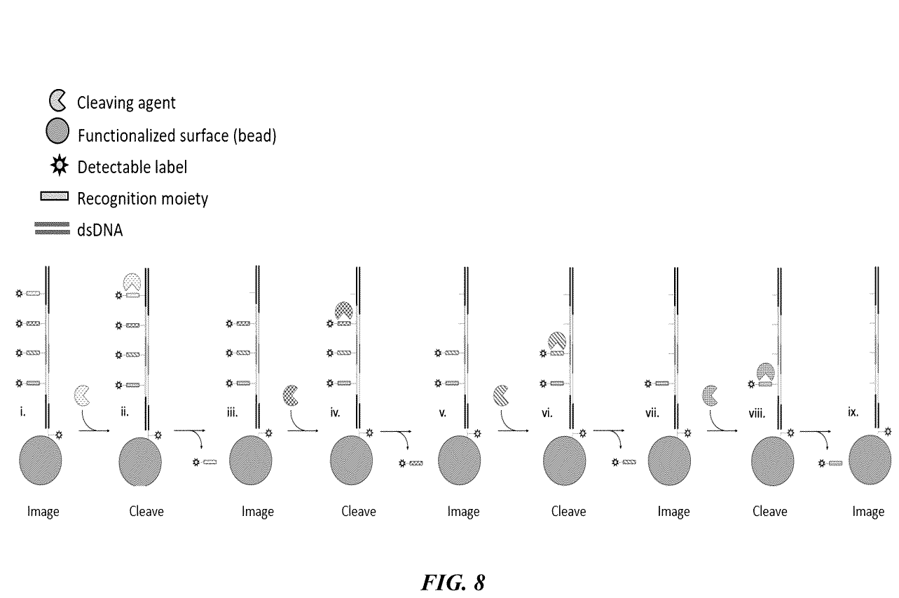

The recognition moiety may comprise a single endonuclease cleavage site type for each identimer class making up the barcode (FIG. 5). The cleavage agents for these molecular barcodes may be proteases (FIG. 8, FIG. 10), nucleases (FIG. 7, FIG. 9), or chemical cleavage agents. Many endonucleases are known in the art to possess orthogonal reactivity with respect to one another. For example, NotI, XhoI, SpeI, and HindIII possess specificity for dsDNA sequences containing GCGGCCGC, CTCGAG, ACTAGT, and AAGCTT, respectively. Therefore, in some embodiments, NotI should not specifically cleave sites recognized by XhoI, SpeI or HindIII with high efficiency. The same is true with respect to each of the nucleases listed, as each should be capable of cleaving (primarily), only their respective substrates in the presence of substrates recognized by the others.

The molecular barcode may have the scaffold portion comprising double stranded DNA, the recognition moiety comprising a nuclease recognition moiety, and the one or more detectable labels comprising a fluorophore. Alternatively, the molecular barcode may have the scaffold portion comprising double stranded DNA, the recognition moiety comprising a protease recognition moiety, and the one or more detectable labels comprising a fluorophore. Further alternatively, the molecular barcode of the first embodiment may have the scaffold portion comprising double stranded DNA, the recognition moiety comprising a chemical cleavage recognition moiety, and the one or more detectable labels comprising a fluorophore.

Non-DNA molecular barcodes may have the scaffolding portion comprising amino acids, peptides, or protein. The recognition moiety may be made of a chemical cleavable moiety and may include more than one detectable label, for example, a combination of two or more different labels. The cleavage agents for these barcodes may be proteases or chemical cleavage agents, which may be added sequentially. Alternatively, the molecular barcode may have the scaffold portion comprising amino acids, the recognition moiety comprising a chemical cleavage recognition moiety, and the one or more detectable labels comprising a fluorophore. Further alternatively, the molecular barcode may have the scaffold portion comprising amino acids, the recognition moiety comprising a protease recognition moiety, and the one or more detectable labels comprising a fluorophore.

The recognition moiety may comprise a chemical linker, peptide, or nucleic acid. Identimers containing peptides of the above recognition motifs belong to different classes; a class is defined by the protease it is identified by.

The one or more of the detectable labels may comprise a fluorophore.

A benefit of the present disclosure is that the molecular barcode may be associated with a material having mutual information with the three-dimensional arrangement of the molecular barcode. Accordingly, the material may be classified by decoding the molecular barcode. For example, a set of one or more molecular barcodes may be introduced into a milieu (e.g., a chemical or biochemical system), wherein at least one molecular barcode in the set has mutual information with a material also present in the milieu. Orthogonally reactive cleaving agents to the set of molecular barcodes may be selectively applied to cleave the molecular barcodes at the cleavage sites that are reactive to the cleaving agents. The cleavage dissociates the detectable labels of identimers from the three-dimensional arrangements according to the orthogonality of the identimers. The dissociation of the detectable labels induces a detectable signal response. The signal response may be used to decode the set of one or more molecular barcodes (i.e., determine the identity and order of the specific identimers). Decoding the set of one or more molecular barcodes reveals the mutual information shared with the material (e.g., the identity and/or concentration of the material). This material may be also linked to the same bead as the molecular barcode used for encoding its identity.

As used herein, “to encode” refers to converting information, data, or classification instructions into a converted format.

As used herein, “to decode” refers to reversing an encoding process to extract information from a converted format.

As used herein, “mutual information” refers to a quantity information obtainable about a first variable through observing a second variable.

As used herein, “orthogonal” refers to a component in a multicomponent system that has chemical reactivity with a particular reagent under a specific set of reaction conditions while at least one other component in the multicomponent system has limited or no reactivity with the reagent, even though all components in the multicomponent system are present in the same milieu.

As used herein, “orthogonal reactivity” refers to a component in a multicomponent system that has chemical reactivity with a particular reagent under a specific set of reaction conditions while at least one or more components in the system does not, even though all the components in the system are present in the same milieu. Likewise, “orthogonally reactive” refers to a material having orthogonal reactivity.

As used herein, an “identimer” is a name given to a single “identifying molecule.” As disclosed herein, a single identimer molecule comprises a set of one or more detectable labels operatively connected to scaffold portion, the scaffold portion comprising a recognition moiety and a cleavage site, prior to their incorporation and encoding of a molecular barcode.

As used herein, an “identimer class” is a name given to a set of one or more identimers having an essentially identical configuration. Thus, identimers in a class should react essentially the same to a stimulus.

“As used herein, an “identimer token” is a tangible instance of an identimer class that has been incorporated into a molecular barcode or has been physically associated with a material.

As used herein, a “recognition moiety” and “recognition portion” are used interchangeably and refer to chemical moieties that are reactive to chemical or enzymatic cleaving agents.

As used herein, a “cleavage site” is the point of cleavage between two disassociated molecules. In some embodiments, a cleavage site is located within the recognition moiety. In some embodiments, a cleavage site is located outside of or distant from, the recognition moiety.

As used herein, a “token” is a thing acting as a visible or tangible representation of information, such as a fact, quality, data, or other form of information.

For example, the material may comprise a test agent operatively coupled to a molecular barcode (collectively referred to as a “test construct”), wherein the three-dimensional arrangement of the molecular barcode has mutual information with the test agent. In this example, decoding the barcode indicates that the test agent is or was present in the milieu with the molecular barcode. Accordingly, test constructs utilizing the barcodes may be used in screening.

For example, methods of screening may include introducing a plurality of test constructs into a milieu, wherein each test construct comprises a test agent operatively coupled to a unique molecular barcode, wherein the three-dimensional arrangement of the molecular barcode has mutual information with the test agent. The plurality of test constructs is screened against a set of one or more targets. The activity of one or more of the test constructs is determined. Orthogonally reactive cleaving agents are selectively applied to the plurality of test constructs to cleave coupled molecular barcodes at the cleavage sites that are reactive to the cleaving agents (i.e., primarily cleaves it at the cleavage sites of only the identimers having recognition moieties that are reactive to the cleaving agents). This dissociates the detectable labels of identimers from the three-dimensional arrangements only according to the orthogonality of the identimers. Therefore, the detectable signal response can be used to decode the barcodes (i.e., identify the barcode identimer sequence, in this example) and thereby identify the corresponding test agents with activity against the set of targets.

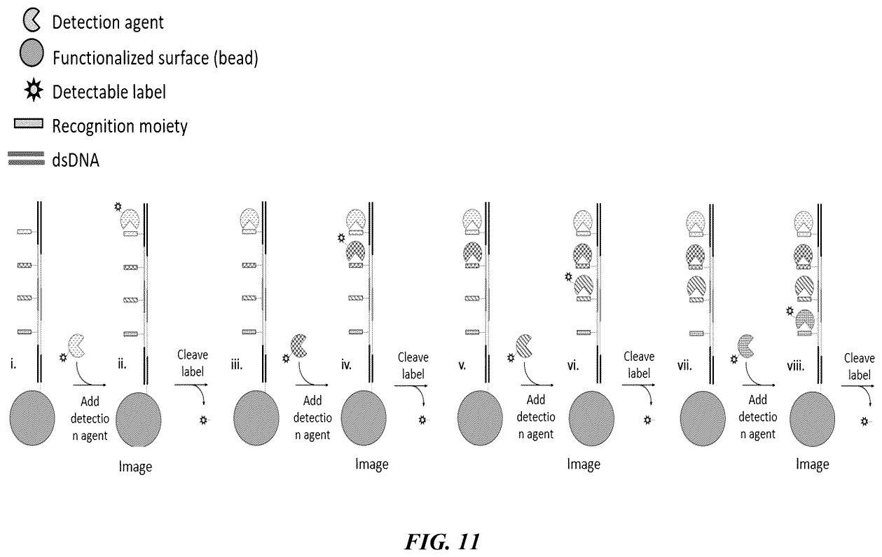

Recognition and cleavage of detectable labels in some molecular barcodes may need to be performed in sequence from the outermost segment toward the bead (e.g., FIG. 7), or barcode information may be lost. Additionally, recognition and cleavage of detectable labels in some molecular barcodes may need to be performed in a known sequence to retain the barcode information (e.g., FIG. 8, FIG. 11). Recognition moiety can be made of protein or a chemically cleavable moiety. Optionally the recognition moiety contains more than one detectable label, for example, combination of two or more different labels.

Beneficially, the detectable signal response may be induced without enrichment. The detectable signal response may be an increase or decrease in signal intensity, such as fluorescence intensity.

Selectively applying orthogonally reactive cleavage agents to the plurality of test constructs may involve applying the cleavage reagents sequentially (e.g., applying a single cleaving agent iteratively or two or more cleaving agents individually and sequentially). Cleavage agents may be different proteases and/or different chemical cleavage agents.

Methods for generating a molecular barcode include providing a set of one or more identimers, each identimer in the set of identimers comprising a set of one or more detectable labels operatively connected to a scaffold portion, the scaffold portion comprising a recognition moiety that is orthogonal to the recognition moiety of at least one identimer in the set of identimers and a cleavage site operatively connected to a set of one or more detectable labels. The identimers may be labeled in a combinatorial way. For example, 10 different quantum dots may be encoded with the same protease site for each identimer in a 7-identimer barcode, which could possibly result encoding 10 million different barcodes or beads. The methods include selecting first and second identimers from the set of identimers and concatenating the first identimer to the second identimer in a three-dimensional arrangement to encode and form a molecular barcode. The method may continue with selecting a identimer from the set of identimers and concatenating it to the molecular barcode to modify the three dimensional arrangement and further encode and form the molecular barcode. This last step may be repeated one to nth times.

Identimer chains may be built by a sequential split-pooling approach, and a different class is added in each round. In some embodiments, a variety of identimer classes may be concurrently used to encode and form molecular barcodes by split-pool ligation to create a combinatorial library of molecular barcodes without reliance on DNA sequencing-based decoding. In some embodiments, individual molecular barcodes may be generated in the presence of other molecular barcodes being formed and encoded simultaneously. Such other molecular barcodes may include Next Generation Sequencing barcodes attached to sites on the bead other than the site of identimer barcode attachment.

In some embodiments, identimers may be used to build combinatorial chains of identimer-based molecular barcodes by splitting and pooling of beads. Essentially, each round of addition adds a unique identimer token to each combinatorial chain. Essentially, each resulting molecular barcode will contain a combination of detectable labels organized according to the three-dimensional arrangement of the identimer tokens incorporated into the molecular barcode.

Identimers may be attached by native chemical ligation, click chemistry, enzymatically by peptide ligases, sortase or SFP synthase for example. Only one class of identimer within a barcode is identified during each cycle of cleavage.

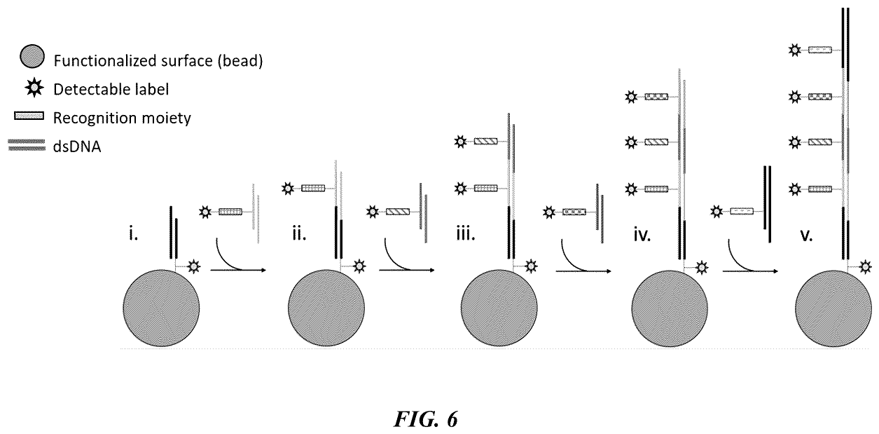

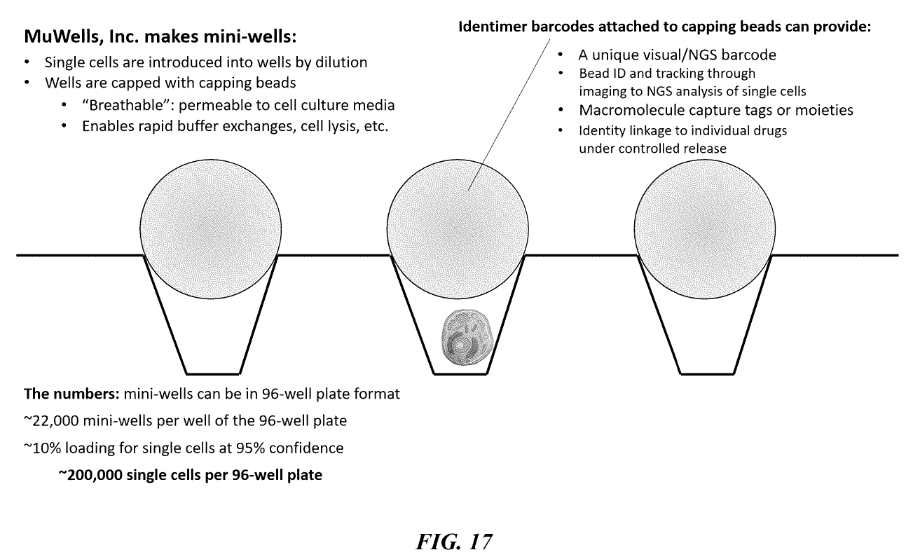

The first identimer may be operatively coupled to a material, such as a surface of a material, such as the surface of a bead. The first identimer may be attached to the bead by using common chemistry. The first identimer may be an oligomer and attached to the bead by its 5′ end (FIG. 6). The bead may be operatively coupled to a test agent. The bead may be a capping bead (FIG. 17). The first identimer may be operatively coupled to the surface of the material by a high-affinity binding protein or a three-armed linker.

Providing a set of one or more identimers may include configuring each identimer in the set of identimers to concatenate to a preceding identimer and to facilitate concatenation of the identimers in a linear three-dimensional arrangement and thereby form and encode the molecular barcode sequentially. Configuring the set of identimers to bind specifically to a preceding identimer may include concatenating by ligation to, extension from, or synthesizing onto, the preceding identimer. For example, identimers comprising double stranded DNA may have overhanging ends for ligation.

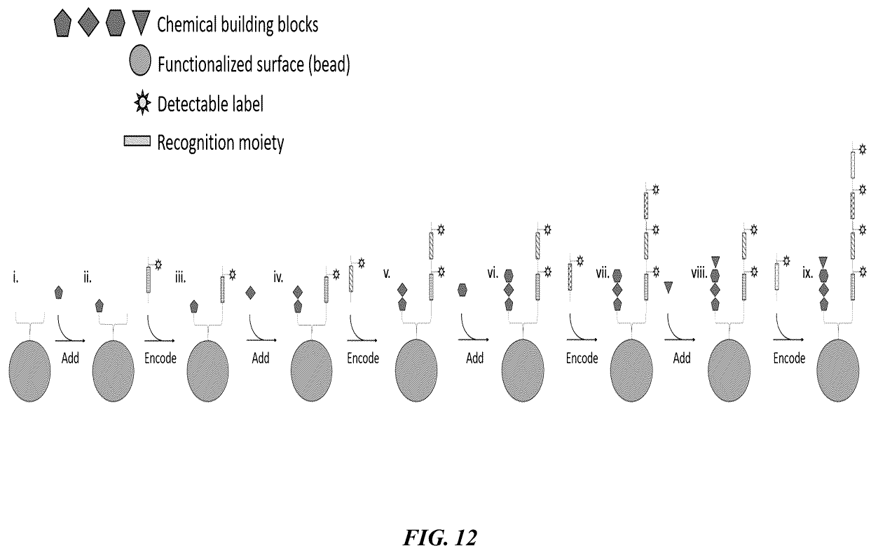

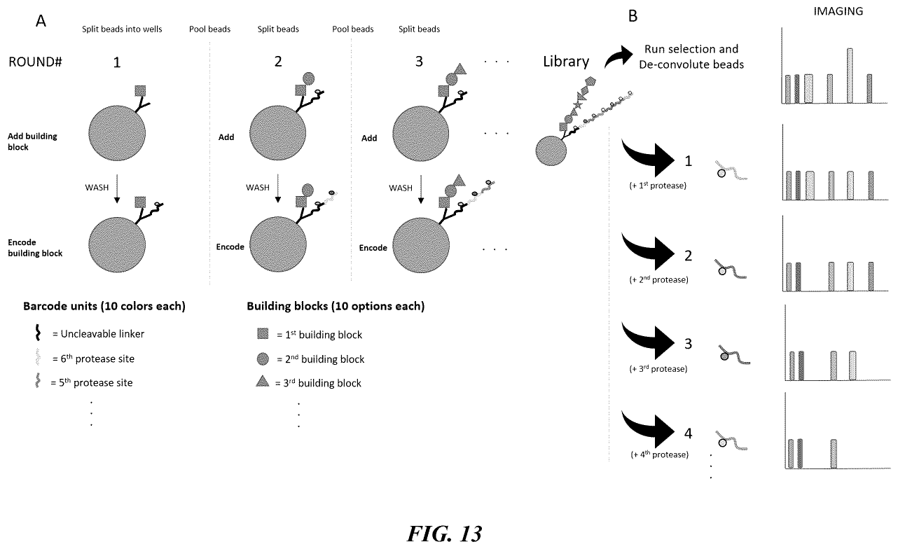

The methods may include providing a set of one or more chemical building blocks, each chemical building block in the set of chemical building blocks being configured to concatenate to a preceding chemical building block; selecting a first chemical building block from the set of chemical building blocks and concatenating it to the first identimer. In other words, following the attachment of a chemical building block, the building block is encoded by a visual barcode segment (FIG. 12 and FIG. 13). The methods may continue with selecting a chemical building block from the set of chemical building blocks configured to concatenate to the preceding chemical building block and concatenating it to the preceding chemical building block and thereby form a series of chemical building blocks. This last step may be repeated one to nth times.

Beneficially, concatenating the series of chemical building blocks may include the use of non-nucleic acid compatible reactions. Thus, reactions may be used to build the molecular barcodes that are not typically available for barcodes that are primarily nucleic acid based.

After a chemical library is built, each bead can display a unique compound whose identity is encoded within the combinatorial visual barcode. The library of compounds may then be selected upon using a variety of known selection schemes. Enrichment of selected compounds over unproductive compounds may not be required following a selection. This barcode type may enable construction and encoding of highly diverse chemical libraries without using DNA. Library members may be distinguished in massively parallel fashion as described previously, using orthogonal cleavage of barcode segments for example.

In some embodiments, molecular barcode libraries may be formed on beads, and the beads can be immobilized on a surface and imaged before and after contact with experimental solutions. Immobilized beads may first be imaged using a standard fluorescence microscope configured with appropriate excitation wavelengths and emission filters to record the combination of visual detectable labels making up each barcode in a given field of view or region of interest.

In place of a standard fluorescence microscope, an inverted fluorescence microscope, any magnified apparatus such as a fluorescence scanner with the appropriate configurations built in, an apparatus that can image fluorescent beads may work. Automated imagers may be useful in a variety of applications. For example, a Molecular Dynamics scanner or Nexcelom scanner for a cell array application using standard 4 fluorophores may be used. For detecting Q-dots, special configurations are required, for example a filter wheel capable of distinguishing all 11 possible Q-dots.

In particular embodiments, after a certain number of fluorophores are attached, one constant color may need to be on all built identimers. For example, if there are 11 distinguishable Q-dots, barcodes may be built using 10 Q-dots and the 11th Q-dot may be used as a standard color to be used as a constant color on all identimers. This may be beneficial as this fluorophore may act as a quantitative standard against which the other fluorophores in any given chain can be compared to in each image. This may allow a user to see how other signals compare relative to that one constant signal and to measure fractions of signals with more accuracy. One of ordinary skill in the art, with the benefit of this disclosure, would understand that other types of internal controls could also be used.

Also disclosed herein is a system for encoding and decoding molecular barcodes. Such systems include a barcode encoding system configured to introduce into a milieu a set of one or more encoded molecular barcodes, in which at least one molecular barcode in a set of encoded molecular barcodes has mutual information with a material. The system further includes a cleavage system configured to selectively apply orthogonally reactive cleaving agents to the milieu to cleave the set of molecular barcodes at the cleavage sites of identimers, in which there are recognition moieties that are reactive to the cleaving agents to dissociate the detectable labels from the three-dimensional arrangement of molecular barcodes in the molecular barcode set according to the orthogonality of the identimers, thereby induce a detectable signal response to decode the set of molecular barcodes. The system further includes a detection system for detecting the detectable signal response from the set of molecular barcodes, and may further include an illumination system configured to selectively convey light to the set of molecular barcodes to induce the detectable signal response.

### Flow Cells

Flow cells were constructed using pre-fabricated plastic covers with adhesive purchased from Microfluidic Chip Shop—cut to enable the generation of 16 individual lanes once mounted on a slide. Flow cell lanes each had approximately a 15-20 μl volume. Poly-L-lysine coated glass slides were used for constructing flow cells; in this way, free primary amines on lysine side chains could be used for modification with NHS-LC-Biotin (at a concentration of 500 μM in NHS Conjugation Buffer at room temperature for at least 1 hour). After biotinylation, flow cell lanes were flushed with 200 μl of 2× Hybridization buffer to prepare them for introduction of oligonucleotide-coated streptavidin beads for immobilization and downstream decoding experiments.

### Molecular Barcode Deconvolution

20 μl of 0.2-1 mg/ml streptavidin beads coated with molecular barcodes were introduced into flow cell lanes in 1× Hybridization buffer. Beads were allowed to settle for at least 1 hour to promote surface attachment. Flow cell lanes were then flushed with 200 μl of 1× Hybridization buffer to remove unbound beads, then 100 μl of 1× CUTSMART® buffer. All identimer decoding experiments were carried out using cleavage solutions containing between 50-500 U of a single restriction enzyme. In some embodiments, a concentration of about 5 U/μl of a single restriction enzyme was used. These solutions were substantially removed of glycerol by first diluting enzyme stocks 2-fold in 1× CUTSMART® buffer (NEB), and buffer exchanging into 1× CUTSMART® buffer via a 7K MWCO Zeba column (commercially available from ThermoFisher, Inc.). Images of uncleaved 1 μm MyOne T1 beads coated with various molecular barcodes were acquired prior to the introduction of the first cleavage agent. Cleavage solutions were introduced into lanes of the flow cell and incubated for 15-30 minutes at 37° C. during each decoding cycle on a heat block. Following each decoding cycle, images of beads were acquired and compiled for downstream analyses.

### Collection of Streptavidin Bead Images

Imaging of streptavidin beads was performed on a Leica Thunder system with a HCX PL FLUOTAR L 40× (NA-0.6) CORR PH2 objective with a Lumencor Spectra X Light Engine (395, 440, 470, 550, 640, 748) and a Leica DFC9000 GTC camera. For image acquisition the following filter sets were utilized (Quad Cube-Ex: 375-407, 462-496, 542-566, 622-654, DC: 415, 500, 572, 660, Em: 420-450, 506-532, 578-610, 666-724 and Y7 cube Ex: 672-748, DC: 760, Em: 765-855). An additional DFT5 fast filter wheel was downstream of the cubes and included the following LP filters: 440, 510, 590, 700 and 100%.

### Image Analysis of Cyclic Decoding of Molecular Barcodes Conjugated to Streptavidin Beads

Images were loaded into a Volocity database and analyzed in the following manner. First, the individual images were made into a time series in the reverse order in which they were acquired. Images were then movement corrected and cropped to the area from the middle of the field to minimize uneven illumination. For each image same size ROI's were drawn around 10 beads and one background area, for calculating background subtractions. Values obtained for each label were subtracted from the next image in the cycle, to clearly identify the fluorophore labels released during each cleavage cycle. For example, in an OCS experiment of three images (uncleaved, cleaved by RE1, and cleaved by RE2), data acquired in the last image (following cleavage by RE2) is subtracted from data acquired in the second to last image (following cleavage by RE1). Data acquired in second to last image (following cleavage by RE1) is subtracted from data acquired in the first image (uncleaved). This enabled accurate identification of signal loss during each cleavage cycle.

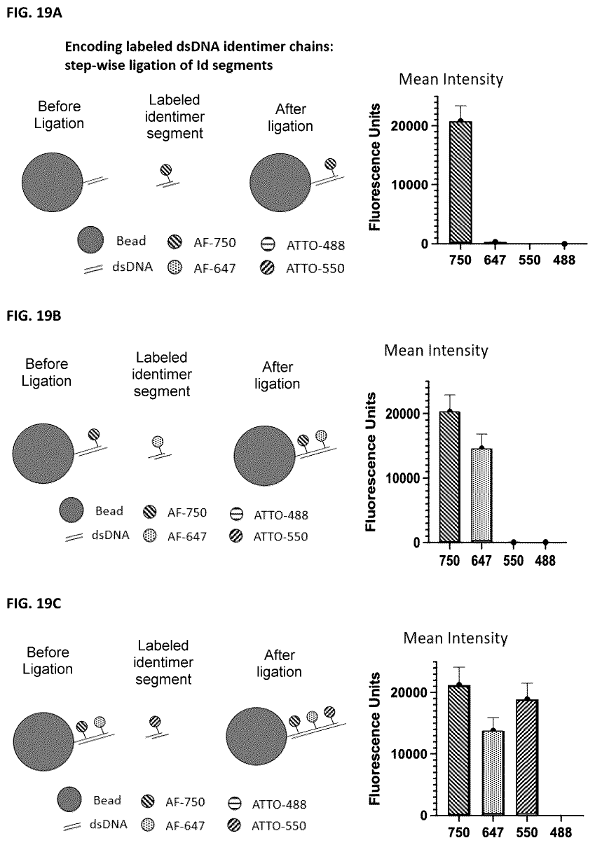

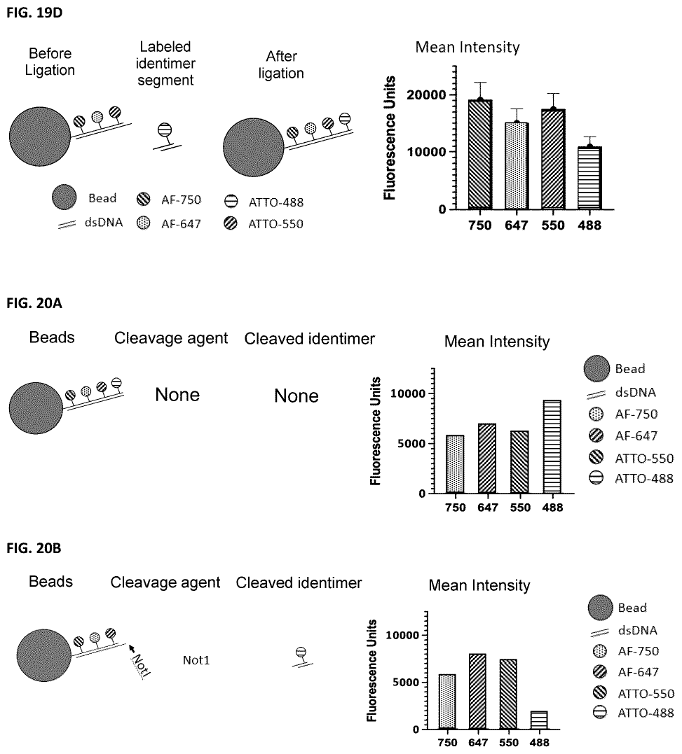

A streptavidin bead is shown in FIG. 19A, containing a first labeled dsDNA identimer segment (Id₁/HindIII-AF750) attached through ligation to an unlabeled dsDNA that is attached to the bead through a biotin moiety. The dsDNA identimer segment, also shown to the right of the bead, contains the AF750 label (AF-750) (downward diagonal stripes) attached to one of the oligos of the DNA duplex through an internal amino-modified base using NHS-AF750. The attached labeled identimer also contains an enzyme-accessible restriction endonuclease site (HindIII), positioned between the attached label and the bead. The Id₁/HindIII-AF750 oligo duplex is attached to the bead through a ligation reaction to an unlabeled oligo (Id₀) that does not contain a recognizable restriction endonuclease site (not cleavable), but does contain a 5′-biotin modification for attachment to the streptavidin bead. Ligation of the dsDNA identimer was performed in-solution, beads were washed, and a sample of the beads were immobilized on a biotin-modified surface in a flow cell for imaging. Attachment of Id₁/HindIII-AF750 to the bead was confirmed by fluorescence imaging in four fluorescence emission channels, 488 nm, 550 nm, 647 nm, and 750 nm, where strong emission signal was observed in only the 750 nm wavelength. Images confirming efficient ligation of the Id₁/HindIII-AF750 dsDNA identimer to the Id₀ oligo duplex are represented in a bar graph plotted using data acquired from all 4 emission wavelengths imaged; 488 nm (horizontal stripes), 550 nm (upward diagonal stripes), 647 nm (dotted), and 750 nm (downward diagonal stripes), and from many beads bearing the same oligos (mean intensity values). The height of the bars in the graph correspond to the relative magnitude of observed emission in all four channels imaged, represented in fluorescence intensity units (FU).

A streptavidin bead is shown in FIG. 19B, containing a second labeled dsDNA identimer segment (Id₂/SpeI-AF647). The second dsDNA identimer segment, also shown to the right of the bead, contains the AF647 label (AF-647) (dotted) attached to one of the oligos of the DNA duplex through an internal amino-modified base using NHS-AF647. The second attached labeled identimer in the chain also contains an enzyme-accessible restriction endonuclease site (SpeI), positioned between the attached label and the bead. The Id2/SpeI-AF647 oligo duplex is attached to the bead through a ligation reaction to the first labeled dsDNA identimer segment (Id₁/HindIII-AF750) duplex. Ligation of the dsDNA identimer was performed in-solution, beads were washed, and a sample of the beads were immobilized on a biotin-modified surface in a flow cell for imaging. Successful formation of the Id₁/HindIII-AF750-Id₂/SpeI-AF647 ligation product on the bead was confirmed by fluorescence imaging in four fluorescence emission channels, 488 nm, 550 nm, 647 nm, and 750 nm, where strong signal was now observed for both the 750 nm and 647 nm emission wavelengths, but not for the 550 nm or 488 nm wavelengths. Images confirming efficient ligation of the Id₂/SpeI-AF647 dsDNA identimer to the Id₁/HindIII-AF750 dsDNA identimer are represented in a bar graph plotted using data acquired from all 4 emission wavelengths imaged, 488 nm (horizontal stripes), 550 nm (upward diagonal stripes), 647 nm (dotted), and 750 nm (downward diagonal stripes), and from many beads bearing the same oligos (mean intensity values). The height of the bars in the graph correspond to the relative magnitude of observed emission in all four channels imaged, represented in fluorescence intensity units (FU).

A streptavidin bead is shown in FIG. 19C, containing a third labeled dsDNA identimer segment (Id₃/XhoI-ATT0550). The third dsDNA identimer segment, also shown to the right of the bead, contains the ATT0550 label (ATTO-550) (upward diagonal stripes) attached to one of the oligos of the DNA duplex through an internal amino-modified base using NHS-ATT0550. The third attached labeled identimer in the chain also contains an enzyme-accessible restriction endonuclease site (XhoI), positioned between the attached label and the bead. The Id₃/XhoI-ATT0550 oligo duplex is attached to the bead through a ligation reaction to the second labeled dsDNA identimer segment (Id₂/XhoI-AF647) duplex. Ligation of the dsDNA identimer was performed in-solution, beads were washed, and a sample of the beads were immobilized on a biotin-modified surface in a flow cell for imaging. Successful formation of the Id₁/HindIII-AF750-Id₂/SpeI-AF647-Id₃/XhoI-ATT0550 ligation product on the bead was confirmed by fluorescence imaging in four fluorescence emission channels, 488 nm, 550 nm, 647 nm, and 750 nm, where strong signal was now observed for the 750 nm, 647 nm and 550 nm emission wavelengths, but not for the 488 nm wavelength. Images confirming efficient ligation of the Id₃/XhoI-ATT0550 dsDNA identimer to the growing identimer chain are represented in a bar graph plotted using data acquired from all 4 emission wavelengths imaged, 488 nm (horizontal stripes), 550 nm (upward diagonal stripes), 647 nm (dotted), and 750 nm (downward diagonal stripes), and from many beads bearing the same oligos (mean intensity values). The height of the bars in the graph correspond to the relative magnitude of observed emission in all four channels imaged, represented in fluorescence intensity units (FU).

A streptavidin bead is shown in FIG. 19D, containing a fourth labeled dsDNA identimer segment (Id₄/NotI-ATT0488). The fourth dsDNA identimer segment, also shown to the right of the bead, contains the ATT0488 label (ATTO-488) (horizontal stripes) attached to one of the oligos of the DNA duplex through an internal amino-modified base using NHS-ATT0488. The fourth attached labeled identimer in the chain also contains an enzyme-accessible restriction endonuclease site (NotI), positioned between the attached label and the bead. The Id₄/NotI-ATT0488 oligo duplex is attached to the bead through a ligation reaction to the third labeled dsDNA identimer segment Id₃/XhoI-AF647) duplex. Ligation of the dsDNA identimer was performed in-solution, beads were washed, and a sample of the beads were immobilized on a biotin-modified surface in a flow cell for imaging. Successful formation of the Id1/HindIII-AF750-Id₂/SpeI-AF647-Id₃/XhoI-ATT0550-Id₄/NotI-ATT0488 ligation product on the bead was confirmed by fluorescence imaging in four fluorescence emission channels, 488 nm, 550 nm, 647 nm, and 750 nm, where strong signal was now observed for the 750 nm, 647 nm 550 nm and 488 nm emission wavelengths. Images confirming efficient ligation of the Id4/NotI-ATT0488 dsDNA identimer to the growing identimer chain are represented in a bar graph plotted using data acquired from all 4 emission wavelengths imaged, 488 nm (horizontal stripes), 550 nm (upward diagonal stripes), 647 nm (dotted), and 750 nm (downward diagonal stripes), and from many beads bearing the same oligos (mean intensity values). The height of the bars in the graph correspond to the relative magnitude of observed emission in all four channels imaged, represented in fluorescence intensity units (FU).

Decoding Labeled dsDNA Identimer Chains; 3 Cleavage Cycles:

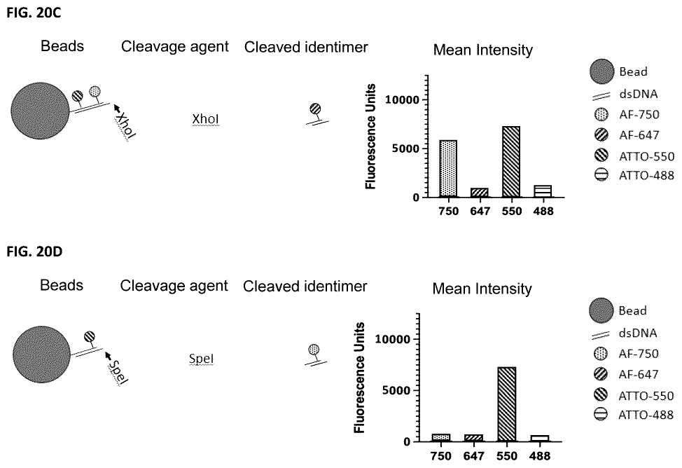

Beads were encoded with a single combination of four different fluorophore labels, where each unique label was attached to a different identimer segment within the dsDNA identimer chain. The dsDNA identimer chain was formed by four rounds of ligation as described previously, and as shown in FIG. 19. For this experiment, the chain sequence was: Id₁/HindIII-ATT0550-Id₂/SpeI-AF750-Id₃/XhoI-AF647-Id₄/NotI-ATT0488. It is therefore to be expected that beads bearing such a dsDNA identimer chain sequence would lose fluorescence signal in the 488 nm channel following exposure to only the NotI enzyme in a first cycle of orthogonal cleavage, then in the 647 nm channel following exposure to only the XhoI enzyme in a second cycle of orthogonal cleavage, and in the 750 nm channel following exposure to only the SpeI enzyme in a third cycle of orthogonal cleavage. Here, beads were immobilized on the surface of a flow cell and subjected to three cycles of orthogonal cleavage sequencing (OCS) using these enzymes in the aforementioned cycling order, with images acquired before and after each cycle. First, un-cleaved beads (FIG. 20A) were imaged in four fluorescence channels; 647 nm (upward diagonal stripes), 550 nm (downward diagonal stripes), 488 nm (horizontal stripes) and 750 nm (dotted), and mean intensity values obtained for each fluorophore were plotted to the right. Signal for all four fluorophores was observed in the acquired images of un-cleaved beads (bar graphs to the right). The flow cell was then placed on a 37 C heat block and exposed to a solution containing 1× CUTSMART® buffer and the NotI enzyme at 5 U/μl concentration for 15 minutes. The flow cell lane was then flushed with 1× CUTSMART® buffer, and the flow cell was returned to the microscope for imaging. Images of beads following exposure to the NotI enzyme (FIG. 20B) were acquired from the same location of the same flow cell lane that was imaged for quantification of un-cleaved beads (although the flow cell was removed from the microscope and placed on a heat block for incubation during each cleavage cycle, it was returned to the microscope and this FOV was maintained throughout all images acquired during the experiment). Beads showed significantly lower signal in the 488 nm channel following exposure to NotI, as shown in FIG. 20B (mean intensity plot to the right); the mean intensity of 488 nm signal emission from beads prior to NotI exposure was around 9,300 FU, and following exposure to NotI this signal dropped to around 2,000 FU. Importantly, signal intensities observed for all other fluorescent labels remained substantially unchanged. To initiate a second cycle of orthogonal cleavage, the flow cell was returned to the 37 C heat block and exposed to a solution containing 1× CUTSMART® buffer and the XhoI enzyme at 5 U/μl concentration for 15 minutes. The flow cell lane was then flushed with 1× CUTSMART® buffer, and the flow cell was returned to the microscope for imaging. Images of beads following exposure to the XhoI enzyme (FIG. 20C) showed a significant reduction in signal emission from the AF647 label. The mean intensity of 647 nm signal emission from beads prior to XhoI exposure was around 8,000 FU, and following exposure to XhoI this signal dropped to around 1,000 FU. Importantly, signal intensities observed for the two un-cleaved fluorescent labels remained substantially unchanged. To initiate a third cycle of orthogonal cleavage, the flow cell was returned to the 37 C heat block and exposed to a solution containing 1× CUTSMART® buffer and the SpeI enzyme at 5 U/μl concentration for 15 minutes. The flow cell lane was then flushed with 1× CUTSMART® buffer, and the flow cell was returned to the microscope for imaging. Images of beads following exposure to the SpeI enzyme showed a significant reduction in signal emission from the AF750 label (FIG. 20D). The mean intensity of 750 nm signal emission from beads prior to SpeI exposure was around 5,500 FU, and following exposure to SpeI this signal dropped to around 400 FU. Importantly, signal intensities observed for the final, un-cleaved fluorescent label (ATT0550) remained substantially unchanged. Exposure of the chain sequence: Id₁/HindIII-ATT0550-Id₂/SpeI-AF750-Id₃/XhoI-AF647-Id₄/NotI-ATT0488 to NotI, then XhoI, followed by SpeI produced the expected decoding results.

Decoding Differentially-Labeled dsDNA Identimer Chains: Single and Mixed Segments

Single-label chains were constructed whereby segments of Id₁/HindIII, Id₂/SpeI, Id₃/XhoI and Id₄/NotI were each differentially labeled with the four fluorophores listed previously (ATT0488, ATT0550, AF647, and AF750) to make a total of 16 different labeled dsDNA identimer segments. One differentially-labeled segment of each type (Id₁/HindIII, Id₂/SpeI, Id₃/XhoI and Id₄/NotI) was selected for encoding a pre-defined chain sequence. The single-label dsDNA identimer chain sequence used here was: Id₁/HindIII-ATT0550-Id₂/SpeI-AF750-Id₃/XhoI-AF647-Id₄/NotI-ATT0488. Streptavidin beads containing the pre-defined single-label chain sequence were constructed by the split-pooling ligation strategy described previously, and immobilized on a biotin-modified surface in one lane of a flow cell for imaging. Many beads in a single FOV were imaged in all four fluorescent channels prior to contact with solutions containing restriction enzymes. Beads were then subjected to three cycles of OCS (NotI in cycle 1, XhoI in cycle 2, and SpeI in cycle 3) as described previously, followed by imaging after each cycle. For each acquisition in the imaging series, data for many beads in a single FOV were averaged, and the same FOV was imaged across all cycles of the OCS experiment. Although Id₁ is cleavable by HindIII, and was labeled with ATT0488, this Id segment was not cleaved here, as signal from a single fluorophore label was easily visualized without needing to cleave this segment. Intensity data obtained by imaging of all four labels before and after each cycle of cleavage was entered into a table, with cycles listed in chronological order. It was clear from the raw data, which fluorophore was cleaved during each OCS cycle. However, to more clearly define the order of labels attached to each Id segment in the chain, data were plotted as sequences obtained from bead “reads”. OCS results obtained for single-label dsDNA identimer chains is displayed in the bar graphs shown in FIG. 21A, where the order of the cycles was reversed for analysis. In this way, images taken following the last cycle of orthogonal cleavage in this experiment (cleavage by SpeI) became the “first” image in the series, images acquired following XhoI cleavage became the “second” image in the series, images acquired following NotI cleavage became the “third” image in the series, and images acquired of un-cleaved beads became the “last” image in the series. While in reverse-chronological order, the intensity data for all four fluorophores was subtracted from the “previous” cycle. The resulting data for all four fluorophores were then plotted for each cycle in single-label dsDNA identimer chain decoding imaging series, as shown in FIG. 21A. In this example, beads were imaged following SpeI cleavage, and strong signal was observed for the only remaining fluorophore on the bead, ATT0550 (downward diagonal stripes), which was attached to Id₁. Observed intensity data for all four fluorophores in images following SpeI cleavage were subtracted from images acquired following XhoI cleavage, to arrive at a clear, single label that was cleaved during the SpeI cleavage cycle, AF750 (light dotted), which was attached to Id₂. Likewise, signals in all four channels observed on beads following XhoI cleavage were subtracted from images acquired following NotI cleavage, to enable observation of a single label that was removed upon XhoI cleavage, AF647 (dark dotted), which was attached to Id₃. Finally, all intensity observed on beads in images following NotI cleavage were subtracted from fluorophore intensities obtained from images acquired of un-cleaved beads, to enable observation of a single label that was removed upon NotI cleavage, ATT0488 (horizontal stripes), which was attached to Id4.

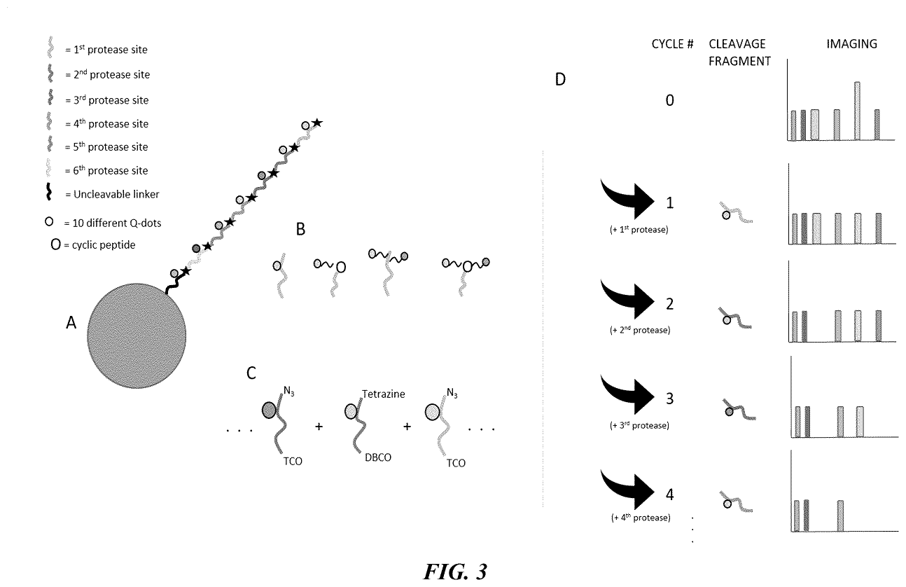

To increase the theoretical diversity of libraries encoded using dsDNA identimer chains, mixed-segment chains were constructed whereby two differentially-labeled dsDNA identimers of a common type were equally mixed at a 1:1 molar ratio in 10 different pre-defined, visually discernable combinations (for example, Id₂/SpeI-ATT0488 was mixed with Id₂/SpeI-AF647) prior to ligation of the segment. 10 fluorophore combinations can be made visually distinguishable with the 4 different labels used; 488/488, 488/550, 488/647, 488/750, 550/550, 550-/647, 550/750, 647/647, 647-/750, and 750/750. One combination of differentially-labeled segments of each type (Id₁/HindIII, Id₂/SpeI, Id₃/XhoI and Id₄/NotI) was selected for encoding a pre-defined chain sequence with mixed labels at each Id segment position in the chain. The mixed-label dsDNA identimer chain sequence used here was: Id₁/HindIII-ATT0550/AF750-Id₂/SpeI-AF647/ATT0488-Id₃/XhoI-ATT0550/AF647-Id₄/NotI-AF750/ATT0488. Streptavidin beads containing the pre-defined mixed-label chain sequence were constructed by the split-pooling ligation strategy described previously, and immobilized on a biotin-modified surface in one lane of a flow cell for imaging. Many beads in a single FOV were imaged in all four fluorescent channels prior to contact with solutions containing restriction enzymes. Beads were then subjected to three cycles of OCS (NotI in cycle 1, XhoI in cycle 2, and SpeI) as described previously, followed by imaging after each cycle. For each acquisition in the imaging series, data for many beads in a single FOV were averaged, and the same FOV was imaged across all cycles of the OCS series. Although Id₁ is cleavable by HindIII, and was used as a 1:1 mixture labeled with both AF750 and ATT0488, this Id segment was not cleaved here, as signal from only two remaining fluorophore labels were easily visualized (following SpeI cleavage) without needing to cleave this segment. Intensity data obtained by imaging of all four labels before and after each cycle of cleavage was entered into a table, with cycles listed in chronological order. It was not initially clear from the raw data which fluorophores were cleaved during each OCS cycle. To more clearly define the order of labels attached to each Id segment in the chain, data were plotted as sequences obtained from bead “reads”, as performed with single-label chain data. OCS results obtained for mixed-label dsDNA identimer chains is displayed in the bar graphs shown in FIG. 21B, where the order of the cycles was reversed for analysis as performed for single-label chain data. In this way, images taken following the last cycle of orthogonal cleavage in this experiment (cleavage by SpeI) became the “first” image in the series, images acquired following XhoI cleavage became the “second” image in the series, images acquired following NotI cleavage became the “third” image in the series, and images acquired of un-cleaved beads became the “last” image in the series. While in reverse-chronological order, the intensity data for all four fluorophores was subtracted from the “previous” cycle. The resulting data for all four fluorophores were then plotted for each cycle in the mixed-label dsDNA identimer chain decoding imaging series, as shown in FIG. 3B. In this example, beads were imaged following SpeI cleavage, and strong signal was observed for the only two remaining fluorophores on the bead, ATT0550 and AF750 (downward diagonal stripes and light dotted bars, respectively), which were the pre-defined combination of labels attached to Id₁. Observed intensity data for all four fluorophores in images following SpeI cleavage were subtracted from images acquired following XhoI cleavage, to arrive at two clearly-resolved labels that were cleaved during the SpeI cleavage cycle, ATT0488 and AF647 (horizontal stripes and dark dotted bars, respectively), which were the pre-defined combination of labels attached to Id₂. Likewise, signals in all four channels observed on beads following XhoI cleavage were subtracted from images acquired following NotI cleavage, to enable observation of the labels that were removed following XhoI cleavage (unfortunately an imaging error made fluorophores attached to Id₃ undistinguishable in this experiment). Finally, all intensity observed on beads in images following NotI cleavage were subtracted from fluorophore intensities obtained from images acquired of un-cleaved beads, to enable identification of the two labels that were removed upon NotI cleavage, ATT0488 and AF750 (horizontal stripes and light dotted bars, respectively), which were the combination of labels that were attached to Id₄.

Decoding dsDNA Identimer Chains: Tracking Individual Beads Over 3 Cycles (4 Images)

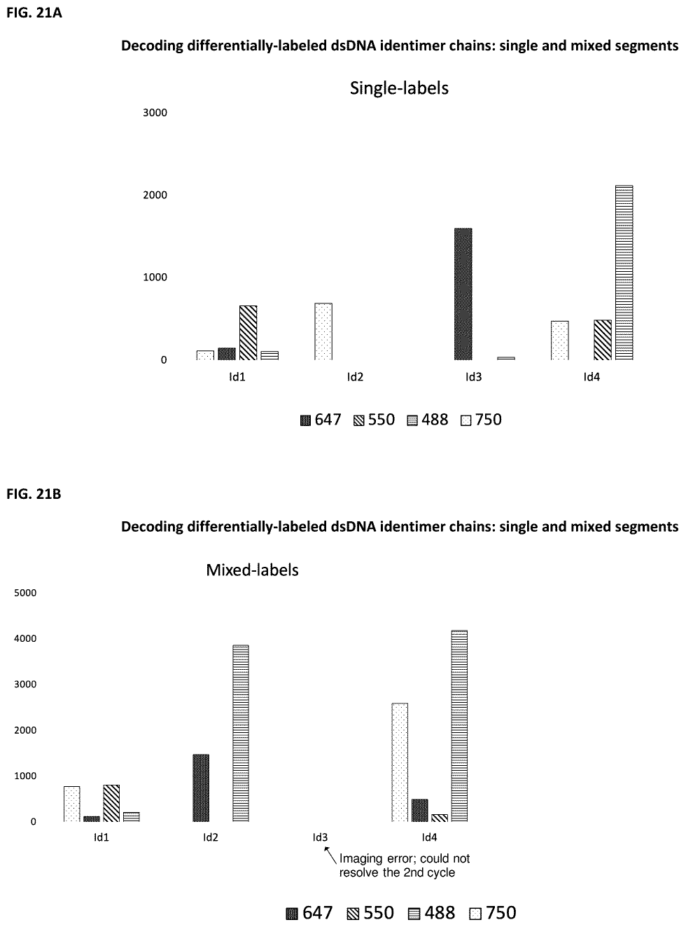

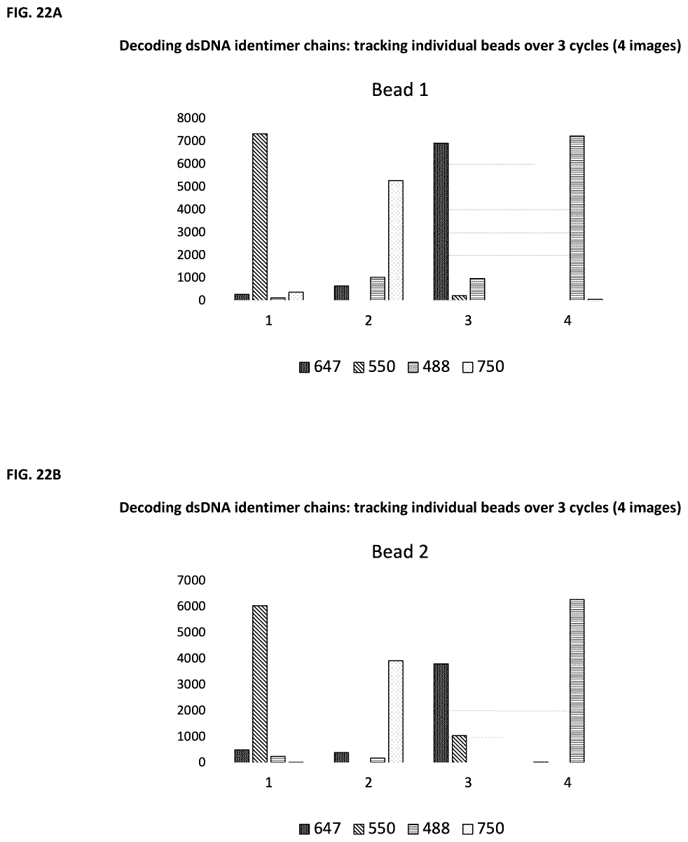

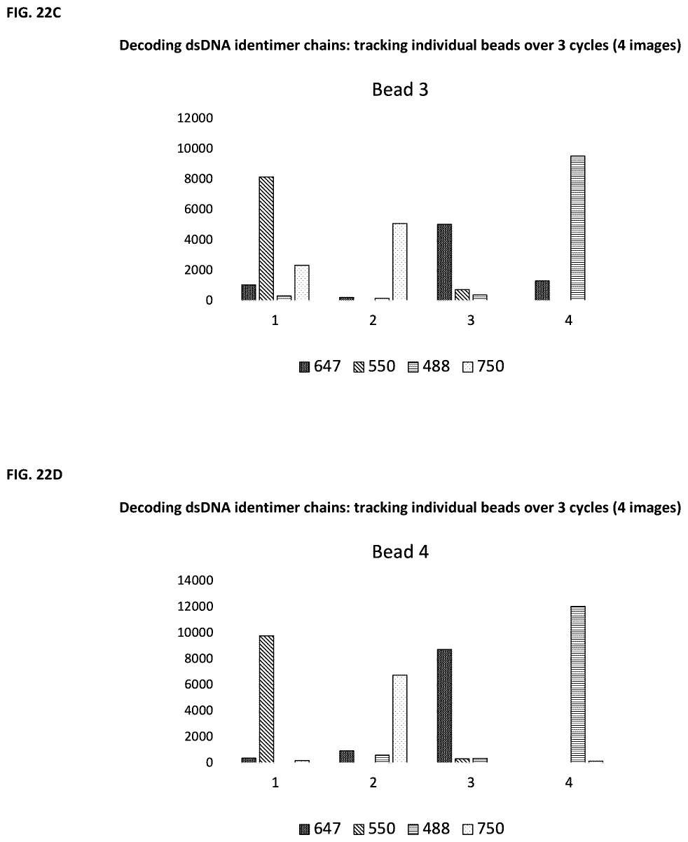

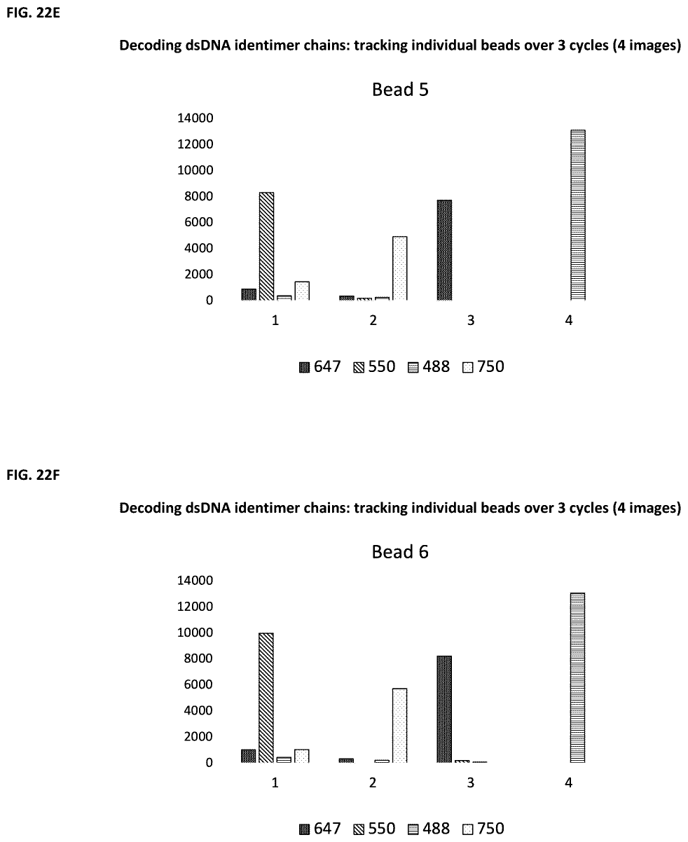

To encode and decode bead libraries of high diversity, accurate tracking of fluorescence intensities from individual beads in multiple fluorescence channels across all cycles of an OCS decoding experiment is required. FIG. 22A through 22F show six individual beads bearing the same single-label identimer chain sequence used to generate data from individual beads in a FOV (Id₁/HindIII-ATT0550-Id₂/SpeI-AF750-Id₃/XhoI-AF647-Id₄/NotI-ATT0488; FIG. 21A), tracked over 3 cycles of an OCS experiment. Beads were immobilized in a flow cell as described previously, and flow cells were transferred to a heat block for incubation with each cleavage agent during its ascribed cycle as described above. Data obtained following imaging of all 4 fluorophores before and after each OCS cycle was plotted following analysis of cycles in reverse chronological order as described above for generation of FIG. 21. All individual beads tracked in this experiment (10 in total; 6 are shown in FIG. 21) demonstrated the expected sequence of fluorophore removal over three OCS cycles. First, ATT0488 (horizontal stripes) was cleaved from the Id₄ segment by NotI, followed by removal of AF647 (dark dotted) from Id₃ by XhoI, followed by removal of AF750 (light dotted) from the Id₂ segment by SpeI, and the Id₁ segment remained as the only labeled segment on the bead following SpeI cleavage, which contained ATT0550 (downward diagonal stripes). Based on these data, the experimental and analytical configuration was ready for simultaneous tracking of many individual beads in massively parallel fashion across at least 3 OCS cycles.

Labeled dsDNA Identimer Chain Structures: Decoding a Library of 256 Labeled Beads

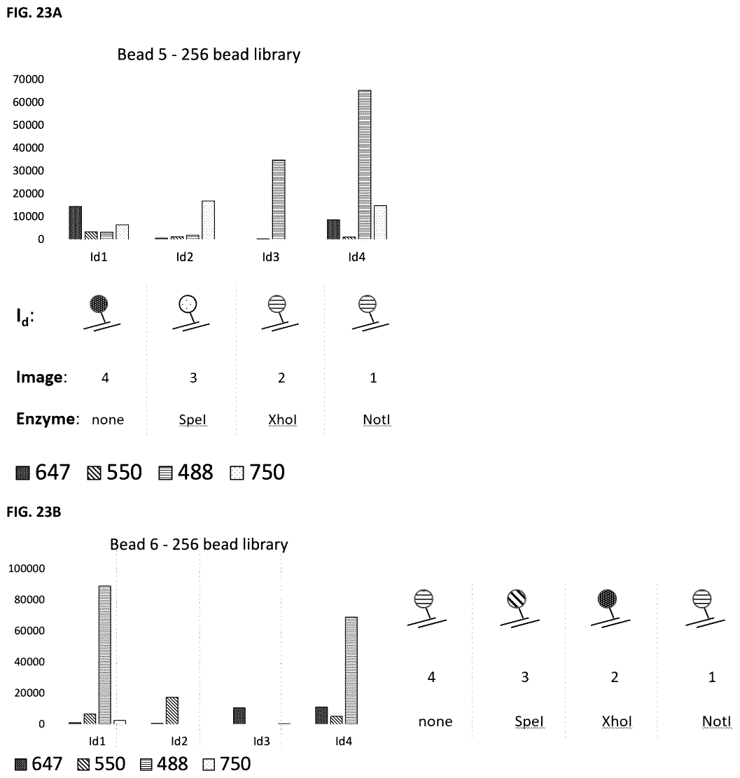

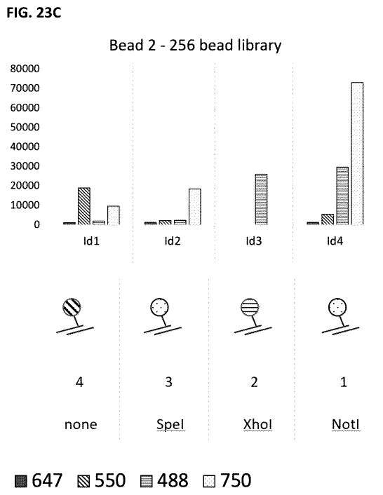

To demonstrate combinatorial encoding of a labeled dsDNA identimer chain-bearing bead library, four different distinguishable labels, ATT0488 (horizontal stripes), ATT0550 (downward diagonal stripes), AF647 (dark dotted), and AF750 (light dotted) were attached to four dsDNA identimer chain segments (Id₁₋₄) to create 4 differentially-labeled options at each dsDNA identimer segment position. A library of 256 different sequences (combinations) of fluorophore-labeled dsDNA identimer chains were constructed over 4 rounds of splitting and pooling by ligation. In brief, beads containing the Id₀ ligation acceptor dsDNA duplex (this duplex does not contain a visual label, but does contain a 5′-biotin modification for attachment to the bead, as well as a 5′ overhang compatible for ligation with Id₁ segments) were split into 4 wells for attachment of a first labeled Id segment (either Id₁/HindIII-ATT0488, Id₁/HindIII-ATT0550, Id₁/HindIII-AF647, or Id₁/HindIII-AF750) by ligation as described in the methods section. Ligations were quenched, beads were then washed and pooled to mix, and beads were then split into 4 wells for attachment of a second labeled Id segment (either Id₂/SpeI-ATT0488, Id₂/SpeI-ATT0550, Id₂/SpeI-AF647, or Id₂/SpeI-AF750) by ligation. Ligations were quenched, beads were then washed and pooled to mix, and beads were then split into 4 wells for attachment of a third labeled Id segment (either Id₃/XhoI-ATT0488, Id₃/XhoI-ATT0550, Id₃/XhoI-AF647, or Id₃/XhoI-AF750) by ligation. Ligations were quenched, beads were then washed and pooled to mix, and beads were then split into 4 wells for attachment of a fourth labeled Id segment (either Id₄/NotI-ATT0488, Id₄/NotI-ATT0550, Id₄/NotI-AF647, or Id₄/NotI-AF750) by ligation. After quenching the final ligation reactions, beads were washed, pooled and immobilized on a biotin-modified surface in a single lane of a flow cell. The beads in the flow cell lane were subjected to 3 cycles of decoding in an OCS workflow. OCS data obtained from individual beads that were tracked throughout the experiment are shown in FIG. 23.

Here beads were imaged prior to restriction enzyme exposure, then imaged following exposure to each individual enzyme (each orthogonal cleavage event) as described previously. In cleavage cycle 1, a solution containing 5 U/μl of the NotI enzyme was flowed into the flow cell lane and incubated for 15 minutes on a heat block set to 37 C. Following cleavage cycle 1 the flow cell was returned to the microscope, and the same FOV used for imaging un-cleaved beads was imaged following exposure to the NotI enzyme. This enabled tracking of the same individual beads across the first two images in the series. This process was repeated with exposure to the XhoI enzyme in cleavage cycle 2, and to the SpeI enzyme in cleavage cycle 3. Intensity values were obtained for all four fluorophore labels attached to the three beads tracked across the OCS experiment shown here, the images were analyzed in reverse chronological order with “previous” cycle subtraction as described above, and values were plotted for cycle-by-cycle visualization of label removal. Three beads tracked across all cleavage cycles during this experiment are shown in FIGS. 23A, 23B, and 23C. Each of the three beads shown in FIG. 23 contained unique dsDNA identimer chain sequences that were easily resolved a) bead 5 sequence: Id₁/HindIII-AF647-Id₂/SpeI-AF750-Id₃/XhoI-ATT0488-Id₄/NotI-ATT0488; b) bead 6 sequence: Id₁/HindIII-ATT0488-Id₂/SpeI-ATT0550-Id₃/XhoI-AF647-Id₄/NotI-ATT0488; and c) bead 2 sequence: Id₁/HindIII-ATT0550-Id₂/SpeI-AF750-Id₃/XhoI-ATT0488-Id₄/NotI-AF750. Below bar graphs displaying data obtained at each imaging step in the series, an illustration of each Id segment released (Id) by each enzyme is shown for the 3 beads tracked. Imaging cycles are separated by dotted vertical lines.

### Labeled DNA Hairpin Structures

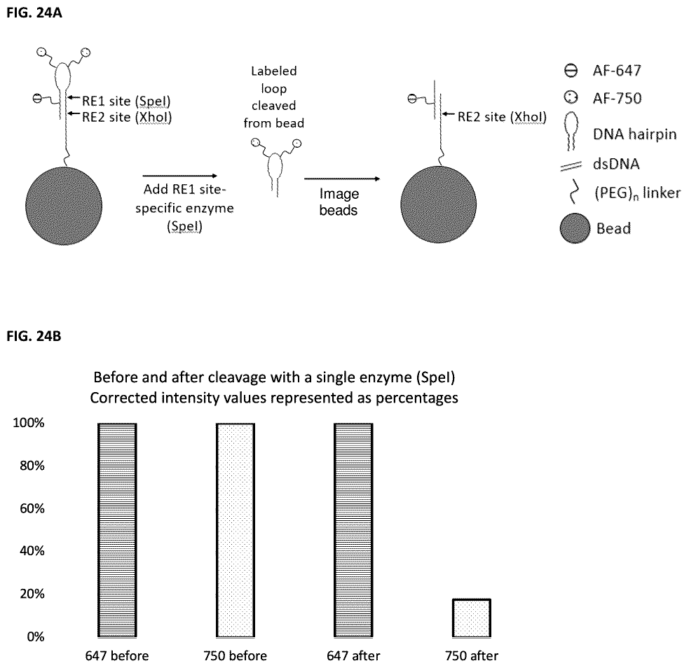

Identimers were constructed by ligation of a pre-hybridized dsDNA duplex (containing a biotin modification on the 5′-end of one oligo strand of the duplex, and a detectable label (AF-647, horizontal stripes in a and b) attached to the other strand of the duplex), and hairpin oligo containing two AF-750 labels (light dotted in a and b). The resulting ligated hairpin contains three detectable labels, a 5′ biotin modification for attachment to a streptavidin bead, and two fully formed and orthogonal restriction sites that flank the AF-647 label. The first of the two restriction sites (RE1 site, specific for cleavage by SpeI) is positioned in between the AF-647 label attached within the stem of the hairpin, and the AF-750 labels attached to the loop of the hairpin. This identimer structure enables building of short identimer chains for decoration of beads with many different combinations of fluorophores attached through orthogonally-cleavable linkers. This hairpin structure ensures that restriction sites remain as double-stranded regions prior to cleavage, because here the complementary strand is fused through the loop of the hairpin structure. Following ligation, this oligo was attached to streptavidin beads, and beads were immobilized on a biotin-modified surface in a flow cell for imaging before and after exposure to one cycle of OCS (FIG. 24A). Values were obtained from many beads in a single field of view, and average intensity values were plotted as percentages. Prior to enzyme exposure, the beads displayed clear, measurable signal from both the AF-647 and AF-750 labels (FIG. 24B before), and these values were therefore 100% of the signal expected from the images after cleavage if no cleavage took place. Upon exposure to the SpeI enzyme, the dual AF-750-labeled hairpin “cap” was liberated from hairpin identimer, as observed in images taken following cleavage (FIG. 24B after). In images taken following cleavage, beads contained slightly greater than 100% of the AF-647 signal observed in images acquired before cleavage, but contained less than 20% of the AF-750 signal following exposure to SpeI. Although the second label of the hairpin is cleavable by XhoI, this fluorophore was clearly distinguishable as the highest remaining signal on the bead, and therefore its cleavage was not necessary for its identification. Additionally, successful cleavage of the structure that remained on the bead has been demonstrated previously (here).

### Encoding Identimer Ring Structures by Step-Wise Ligation

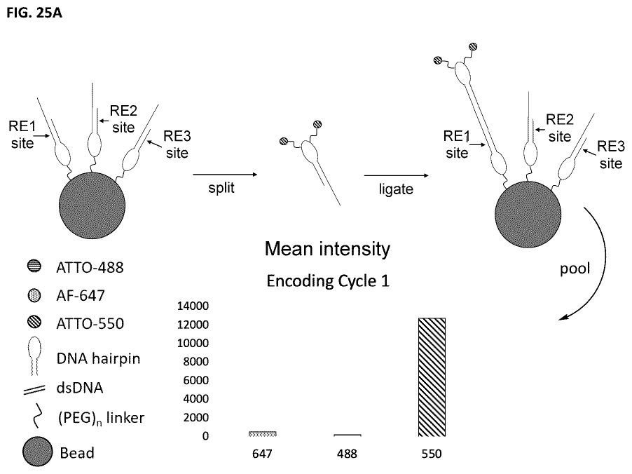

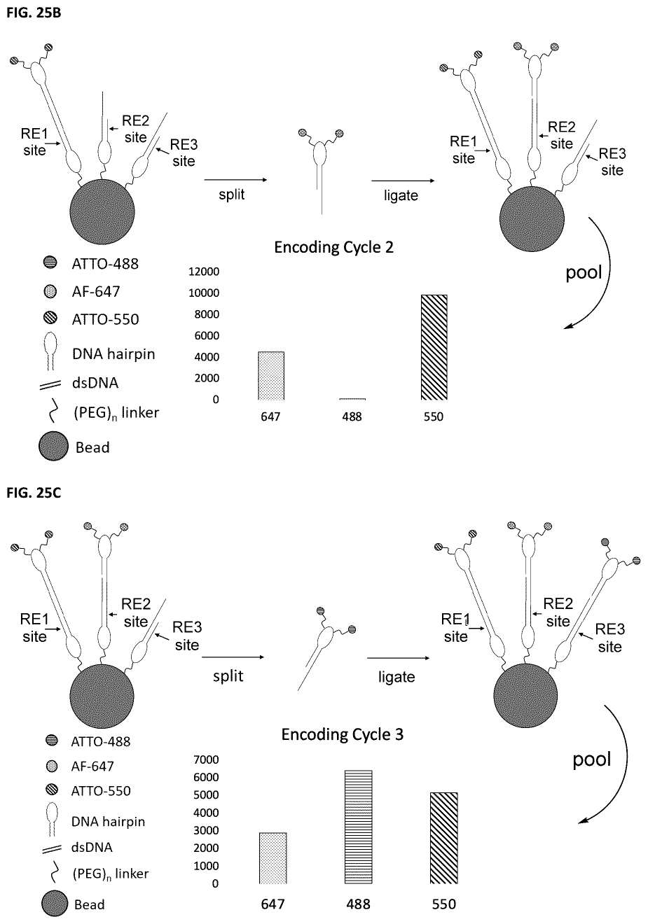

A streptavidin bead is shown in FIG. 25A, containing a first labeled DNA hairpin that is attached through ligation to a first unlabeled DNA hairpin that is attached to the bead through a biotin moiety. The first labeled DNA hairpin generates a labeled identimer segment upon templated ligation with the first ligation acceptor DNA hairpin oligo attached to the bead, as a ssDNA ring containing an encoded restriction site within the dsDNA region. Ligation of each labeled DNA hairpin is designed to be specific to only one acceptor hairpin oligo, enabling orthogonal, step-wise ligation of one labeled hairpin oligo at a time. This allows for a splitting and pooling approach to be taken when constructing libraries of beads containing identimers composed of DNA rings. To demonstrate step-wise construction of DNA ring identimers on beads, three labeled DNA hairpins were used in three rounds of encoding, to mimic a splitting and pooling workflow. The first hairpin, containing the ATT0550 label (downward diagonal stripes) was attached to one of the three acceptor oligos on the bead. The newly formed, labeled DNA ring identimer also contains an enzyme-accessible restriction endonuclease site (SpeI), positioned between the attached labels and the bead. Ligation of the ATT0550 label to the bead was confirmed by fluorescence imaging in three fluorescence emission channels, 488 nm, 550 nm, and 647 nm, where strong emission signal was observed in only the 550 nm wavelength. Images confirming efficient ligation are represented in a bar graph plotted using data acquired from all 3 emission wavelengths imaged; 488 nm (horizontal stripes), 550 nm (downward diagonal stripes), 647 nm (dotted), and from many beads bearing the same oligos (mean intensity values). The height of the bars in the graph correspond to the relative magnitude of observed emission in all three channels imaged, represented in fluorescence intensity units (FU). FIG. 25B shows a second labeled DNA hairpin, containing the AF-647 label (dotted) that was orthogonally attached through ligation to the second of the three acceptor oligos on the bead. The newly formed, labeled DNA ring identimer also contains an enzyme-accessible restriction endonuclease site (XhoI), positioned between the attached labels and the bead. Ligation of the AF-647 label to the bead was confirmed by fluorescence imaging in three fluorescence emission channels, 488 nm, 550 nm, and 647 nm, where strong emission signal was now observed for both the ATTO-550 label and for the AF-647 label. Images confirming efficient ligation are represented in a bar graph plotted using data acquired from all 3 emission wavelengths imaged; 488 nm (horizontal stripes), 550 nm (downward diagonal stripes), 647 nm (dotted), and from many beads bearing the same oligos (mean intensity values). The height of the bars in the graph correspond to the relative magnitude of observed emission in all three channels imaged, represented in fluorescence intensity units (FU). FIG. 25C shows a third labeled DNA hairpin, containing the ATTO-488 label (horizontal stripes) that was orthogonally attached through ligation to the last (third) of the three acceptor oligos on the bead. The newly formed, labeled DNA ring identimer also contains an enzyme-accessible restriction endonuclease site (NotI), positioned between the attached labels and the bead. Ligation of the ATTO-488 label to the bead was confirmed by fluorescence imaging in three fluorescence emission channels, 488 nm, 550 nm, and 647 nm, where strong emission signal was now observed for all three labels (ATTO-550, AF-647, and ATTO-488). Images confirming efficient ligation are represented in a bar graph plotted using data acquired from all 3 emission wavelengths imaged; 488 nm (horizontal stripes), 550 nm (downward diagonal stripes), 647 nm (dotted), and from many beads bearing the same oligos (mean intensity values). The height of the bars in the graph correspond to the relative magnitude of observed emission in all three channels imaged, represented in fluorescence intensity units (FU).

### Decoding Identimer Ring Structures

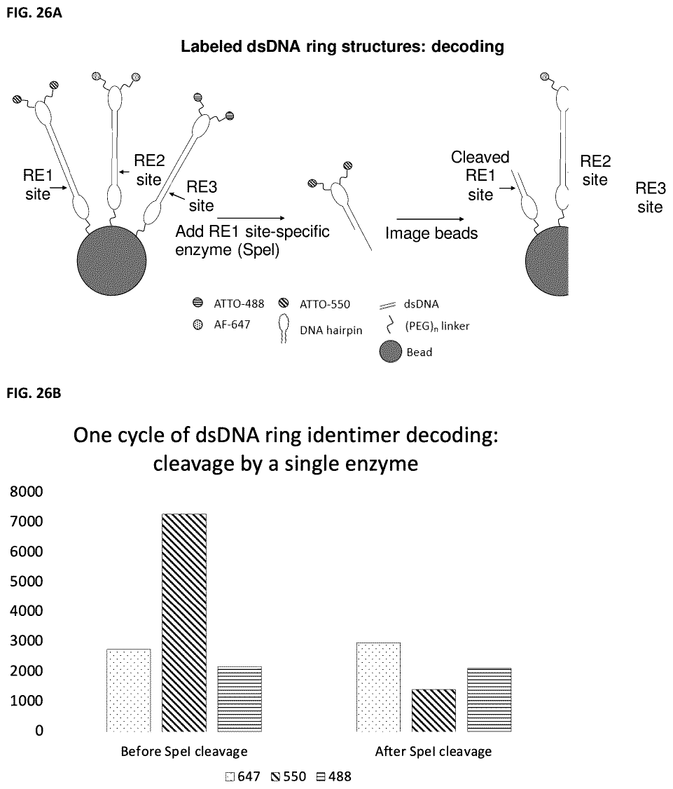

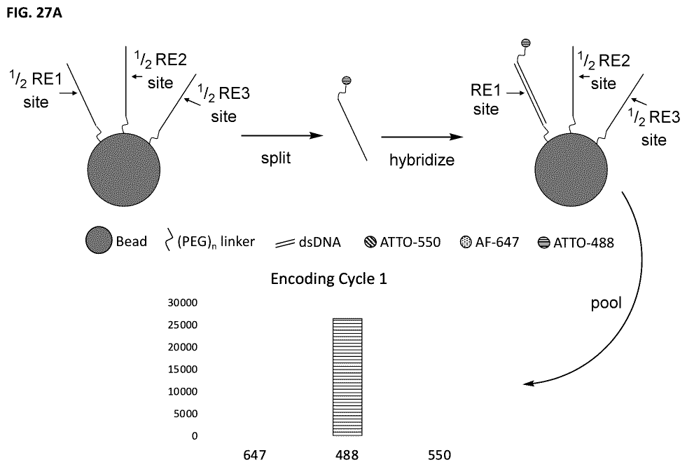

To confirm that labeled identimer ring structures generated here are functional in OCS workflows, beads subjected to three rounds of differentially-labeled DNA ring identimer construction were exposed to a single cycle of orthogonal cleavage. The same beads constructed in (FIG. 25) were immobilized on a biotin-modified surface in a flow cell, and used here. These beads contained three different ring identimers, Id₁/SpeI-ATTO-550, Id₂/XhoI-AF-647, and Id₃/NotI-ATTO-488 (FIG. 26A). Beads were imaged before and after exposure to the SpeI enzyme. Following exposure to SpeI, efficient cleavage of the ATTO-550 label from the bead was confirmed by fluorescence imaging in three fluorescence emission channels, 488 nm, 550 nm, and 647 nm, where a significant loss of signal was observed for the ATTO-550 label relative to signals obtained for the two other labels. Images confirming efficient SpeI cleavage are represented in a bar graph (FIG. 26B) plotted using data acquired from all 3 emission wavelengths imaged; 488 nm (horizontal stripes), 550 nm (downward diagonal stripes), 647 nm (dotted), and from many beads bearing the same oligos (mean intensity values). The height of the bars in the graph correspond to the relative magnitude of observed emission in all three channels imaged, represented in fluorescence intensity units (FU).

### Encoding Identimers by Step-Wise Hybridization

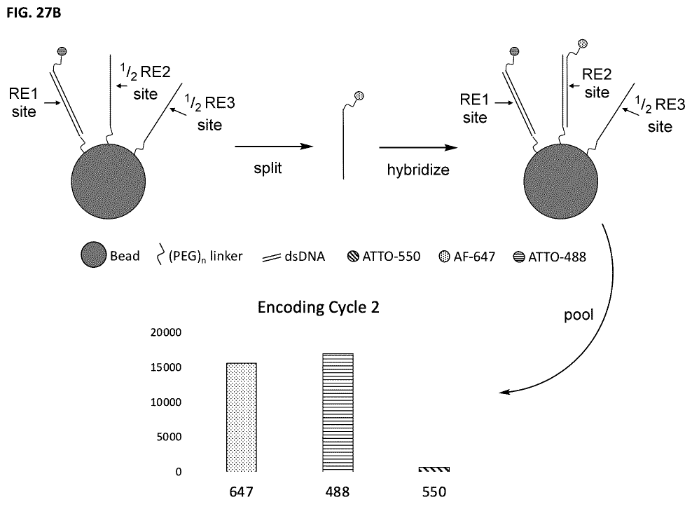

A streptavidin bead is shown in FIG. 27A, containing a first labeled ssDNA that is hybridized to a first unlabeled ssDNA that is attached to the bead through a biotin moiety. The first labeled DNA generates a labeled identimer segment upon templated hybridization with the first of three orthogonal complement strands attached to the bead, as a dsDNA duplex containing an encoded restriction site within the dsDNA region. One half of the required restriction enzyme recognition sequence is encoded in each complementary strand. Hybridization of each labeled ssDNA is designed to be specific to only one complementary oligo on the bead, enabling orthogonal, step-wise hybridization of one labeled ssDNA oligo at a time. This allows for a splitting and pooling approach to be taken when constructing libraries of beads containing identimers composed of labeled hybridized dsDNA. To demonstrate step-wise construction of hybridized identimers on beads, three labeled ssDNA strands were used in three rounds of encoding, to mimic a splitting and pooling workflow. The first ssDNA oligo, containing the ATTO-488 label (horizontal stripes) was hybridized to one of the three complementary oligos on the bead. The newly formed, labeled dsDNA hybridized identimer also contains an enzyme-accessible restriction endonuclease site (NotI), positioned between the attached label and the bead. Ligation of the ATTO-488 label to the bead was confirmed by fluorescence imaging in three fluorescence emission channels, 488 nm, 550 nm, and 647 nm, where strong emission signal was observed in only the 488 nm wavelength. Images confirming efficient hybridization are represented in a bar graph plotted using data acquired from all 3 emission wavelengths imaged; 488 nm (horizontal stripes), 550 nm (downward diagonal stripes), 647 nm (dotted), and from many beads bearing the same oligos (mean intensity values). The height of the bars in the graph correspond to the relative magnitude of observed emission in all three channels imaged, represented in fluorescence intensity units (FU). FIG. 27B shows a second labeled ssDNA oligo, containing the AF-647 label (dotted) that was orthogonally hybridized to the second of the three complementary oligos on the bead. The newly formed, labeled dsDNA hybridized identimer also contains an enzyme-accessible restriction endonuclease site (SpeI), positioned between the attached label and the bead. Hybridization of the AF-647 label to the bead was confirmed by fluorescence imaging in three fluorescence emission channels, 488 nm, 550 nm, and 647 nm, where strong emission signal was now observed for both the ATTO-488 label and for the AF-647 label. Images confirming efficient hybridization are represented in a bar graph plotted using data acquired from all 3 emission wavelengths imaged; 488 nm (horizontal stripes), 550 nm (downward diagonal stripes), 647 nm (dotted), and from many beads bearing the same oligos (mean intensity values). The height of the bars in the graph correspond to the relative magnitude of observed emission in all three channels imaged, represented in fluorescence intensity units (FU). FIG. 27C shows a third labeled ssDNA oligo, containing the ATTO-550 label (downward diagonal stripes) that was orthogonally hybridized to the last (third) of the three complementary oligos on the bead. The newly formed, dsDNA identimer also contains an enzyme-accessible restriction endonuclease site (XhoI), positioned between the attached labels and the bead. Ligation of the ATTO-550 label to the bead was confirmed by fluorescence imaging in three fluorescence emission channels, 488 nm, 550 nm, and 647 nm, where strong emission signal was now observed for all three labels (ATTO-550, AF-647, and ATTO-488). Images confirming efficient hybridization are represented in a bar graph plotted using data acquired from all 3 emission wavelengths imaged; 488 nm (horizontal stripes), 550 nm (downward diagonal stripes), 647 nm (dotted), and from many beads bearing the same oligos (mean intensity values). The height of the bars in the graph correspond to the relative magnitude of observed emission in all three channels imaged, represented in fluorescence intensity units (FU).

### Decoding Hybridized Identimers

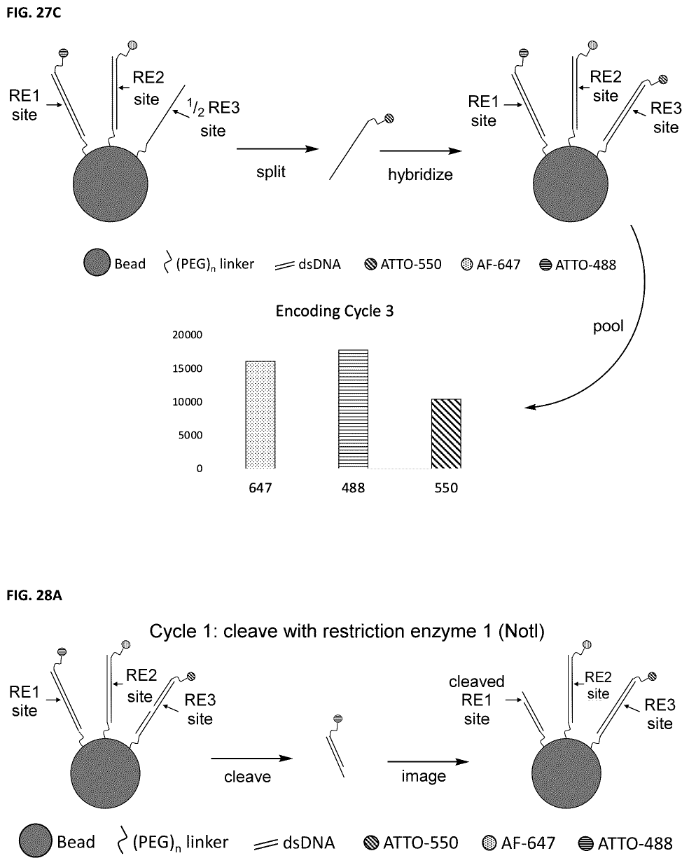

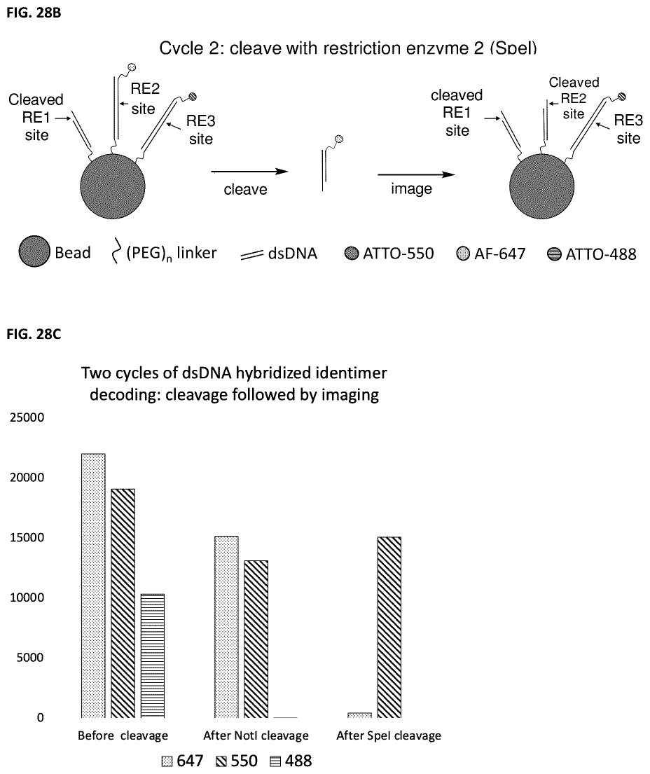

To confirm that labeled identimer structures generated by hybridization here are functional in OCS workflows, beads subjected to three rounds of differentially-labeled ssDNA identimer construction by hybridization were exposed to two cycles of orthogonal cleavage. The same beads constructed in (FIG. 27) were immobilized on a biotin-modified surface in a flow cell, and used here. These beads contained three different hybridized identimers, Id₁/NotI-ATTO-488, Id₂/SpeI-AF-647, and Id₃/XhoI-ATTO-550. Beads were imaged before and after cycle-by-cycle exposure to two enzymes in a known order (first NotI, then SpeI) as described previously (FIGS. 28A and 28B). Following exposure to NotI, efficient cleavage of the ATTO-488 label from the bead was confirmed by fluorescence imaging in three fluorescence emission channels, 488 nm, 550 nm, and 647 nm, where a significant loss of signal was observed for the ATTO-488 label relative to signals obtained for the two other labels. Images confirming efficient NotI cleavage are represented in a bar graph (FIG. 28C) plotted using data acquired from all 3 emission wavelengths imaged; 488 nm (horizontal stripes), 550 nm (downward diagonal stripes), 647 nm (dotted), and from many beads bearing the same oligos (mean intensity values). The height of the bars in the graph correspond to the relative magnitude of observed emission in all three channels imaged, represented in fluorescence intensity units (FU). The beads were then exposed to a second cycle of orthogonal cleavage by the SpeI enzyme, and imaged again. Following exposure to SpeI, efficient cleavage of the AF-647 label from the bead was confirmed by fluorescence imaging in three fluorescence emission channels, 488 nm, 550 nm, and 647 nm, where a significant loss of signal was observed for the AF-647 label relative to beads before SpeI cleavage, and relative to the remaining signal on the bead (ATTO-550). Images confirming efficient SpeI cleavage are represented in a bar graph (FIG. 100) plotted using data acquired from all 3 emission wavelengths imaged; 488 nm (horizontal stripes), 550 nm (downward diagonal stripes), 647 nm (dotted), and from many beads bearing the same oligos (mean intensity values). The height of the bars in the graph correspond to the relative magnitude of observed emission in all three channels imaged, represented in fluorescence intensity units (FU).

### EXAMPLES

The following examples further describe and demonstrate use of embodiments of the disclosed molecular barcode. The examples are given solely for the purpose of illustration and are not to be construed as limiting use of the molecular barcode. Many variations of these examples are possible without departing from the spirit and scope of the present disclosure.

### Example 1 Fluorophore/PROTEASEsite/Non-DNA (FPND) Identimer Class-Based Molecular Barcodes

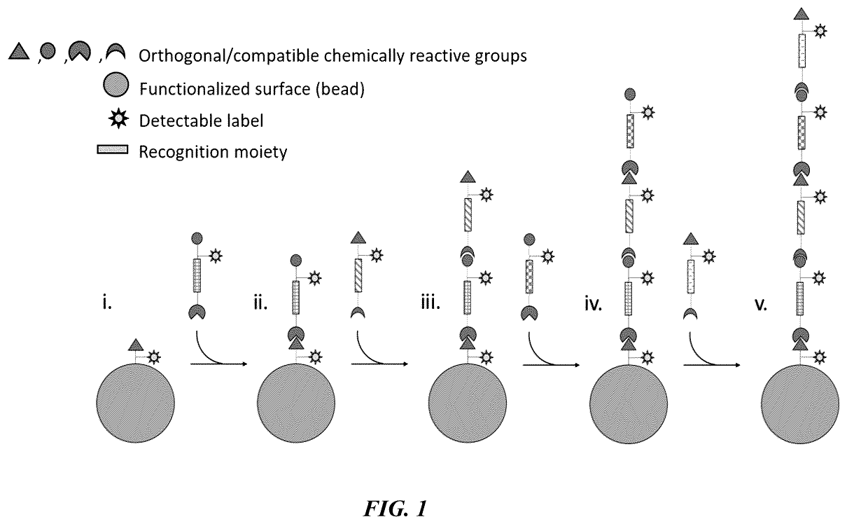

Example 1 illustrates the use of Fluorophore/PROTEASEsite/Non-DNA(FPND) identimers in molecular barcodes for in situ labeling of a material designated for visual decoding (FIGS. 1-3, 15, 16). Generally, FPND identimers comprise fluorophore-based detectable labels operatively connected to polypeptide-based scaffold portions wherein the cleavage site is an orthogonally reactive protease cleavage site and the recognition moiety is a protease cleavage site sequence or a protease recognition sequence of the orthogonally reactive protease.

To confirm the encoding and decoding capacity of FPND identimer-based molecular barcodes a four-cycle orthogonal protease cleavage experiment, may be performed (Experiment 1). Here, a variety of FPND identimer classes may be used concurrently to encode and form molecular barcodes by split-pool ligation to create a molecular barcode combinatorial library without reliance on DNA sequencing-based decoding.

Each molecular barcode in Experiment 1 comprises a set of five identimer tokens (Id₁₋₅) derived from one or more FPND identimer classes. Each Id₁ token is derived from a non-cleavable FPND identimer class and each of the Id₂, Id₃, Id₄, and Id₅ tokens are derived from cleavable FPND identimer classes. Each cleavable FPND identimer is configured to react orthogonally to at least one protease selected from a Tobacco etch virus protease (TEVp), a tobacco vein mottling virus protease (TVMVp), a turnip mosaic virus protease (TUMVp), and a sunflower mild mosaic virus protease (SuMMVp). For example, TEVp is known in the art to have no known off-target substrates in the human proteome and may be used as an orthogonally reactive cleaving agent. And it is known in the art that TEVp, TVMPp1, TUMVp2, and SuMMVp, have been implemented in orthogonal protease regimes. Thus, TEVp should not cleave a molecular barcode at cleavage sites recognized by TVMVp, TUMVp or SuMMV with high efficiency. In other words, each protease should be capable of cleaving a molecular barcode at the cleavage sites of identimers having recognition moieties comprising the respective protease's cleavage site sequence in the presence of substrates recognized by the other proteases. Factor Xa could also be used. Factor Xa cleaves after the arginine residue in its preferred cleavage site Ile-(Glu or Asp)-Gly-Arg. It will not cleave a site followed by a proline or arginine.

In Experiment 1, the FPND identimer scaffold portions comprise a peptide fragment covalently linked through its N- and C-terminal ends to linker reagents containing chemically reactive groups and the detectable labels comprise one or more Q-dots. Q-dot fluorophores are added, respectively, to Id₁, Id₂, Id₃, Id₄, and Id₅ to the modified azide-bearing amino acid in the peptide fragments containing the proteolytic sites. The combination of fluorophores displayed on any formed FPND identimer barcodes in the library (comprising Id₁, Id₂, Id₃, Id₄, and Id₅ tokens as shown here) may or may not have contiguous emission spectra, as the three-dimensional arrangement of the FPND Identimer tokens within the molecular barcodes making up the library will vary.

Limit of detection, specificity, and precision experiments (in triplicate) of proteases acting as cleaving agents may be performed to establish the reproducibility of protease orthogonal reactivity to the FPND Identimer-based molecular barcode.

### A. FPND Identimer Concatenation

As previously described, FPND Identimers can contain orthogonal click chemistry modifications attached at their N- and C-terminal ends. In Experiment, 1, each Id₁, Id₃, and Id₅ identimer class comprises a TCO group at their N-terminal ends, while each Id₂ and Id₄ identimer class comprises a methyltetrazine group at their C-terminal ends. C-termini of Id₁ and Id₃ identimer classes comprise a DBCO group and the Id₂ and Id₄ identimer classes comprise an azide group at their N-terminal ends.

The first identimer attachment (addition of Id₁ for attachment to methyltetrazine-modified beads) is a 45-minute reaction, with incubation at 37° C. Beads are maintained at 1 mg/ml during all reactions, and identimer units are used at a 10 μM concentration for all subsequent additions. Following three wash steps in 1×PBS, the beads are subjected to free TCO in higher concentration (100 μM), to ensure that all methyltetrazine sites are saturated. Following three wash steps in 1×PBS, the beads are ready for addition of the second identimer unit (Id₂). The same reaction conditions are repeated for attachment of Id₂, as well as for each subsequent identimer addition (i.e., Id₃, Id₄, and Id₅) to a FPND molecular barcode. Due to the reaction kinetics of the listed click reactions, following each identimer addition, the reaction is “chased” by addition of the appropriate click reactive group to ensure all reactive sites used for identimer attachment are saturated prior to addition of the next identimer.

### B. Preparation of Each FPND Identimer Library Class

The recognition portions of the Id₁₋₅ identimer classes comprise, respectively, the following peptide sequences: Id₁ is not cleavable and contains no cleavage site; Id₂ comprises KGGSGGGSACVYHQSGGAzGGSC (SEQ ID NO: 1) containing the TUMV cleavage site; Id₃ comprises KGGSGGGSEEIHLQSGGGAzGGSC (SEQ ID NO: 2) containing the SuMMV cleavage site; Id₄ comprises KGGSGGGSETVRFQGGGAzGGSC (SEQ ID NO: 3) containing the TVMV cleavage site; and, Id₅ comprises KGGSGGGSENLYFQSGGAzGGSC (SEQ ID NO: 4) containing the TEV cleavage site.

Generally, FPND Identimers may be generated by first modifying the peptide (through an azide bearing residue installed internally near the C-terminus of all peptides) using DBCO-modified fluorophores for visualization. To accomplish this, 40 μM samples of each peptide can be mixed with a designated DBCO-modified fluorophore at 200 μM, and allowed to react in 1×PBS at 37° C. for at least 3 hours; this reaction can be left overnight at room temperature as well. Fluorophore-modified peptides may then be purified using HPLC, dialysis or desalting columns to remove excess (and any unreacted) DBCO-fluorophore reagent, and to exchange the peptides into NHS-compatible reaction buffer (100 mM Sodium Phosphate buffer, pH 8.5, supplemented with 80 mM KCl). Labeled peptides may also be precipitated using a variety of methods known in the art, and resuspended in NHS-compatible reaction buffer. Labeled peptides may then be brought to a 20 μM concentration in NHS-compatible reaction buffer, and mixed with 100 μM of their designated NHS reagent (NHS-PEG₄-TCO for Id₁, Id₃, and Id₅ peptide fragments; NHS-PEG₄-Azide for Id₂ and Id₄) to enable conjugation overnight at room temperature. N-terminally (singly) click-modified labeled peptides can be purified using HPLC, dialysis or desalting columns to remove excess (and any unreacted) NHS-containing reagent, and to exchange the peptides into 1×PBS. Labeled and singly modified peptides may also be precipitated using a variety of methods known in the art, and resuspended in 1×PBS buffer. Labeled and singly modified peptides may then be brought to a 20 μM concentration in 1×PBS and mixed with 100 μM of their designated maleimide reagent (Maleimide-PEG₄-DBCO for Id₁, Id₃, and Id₅ peptide fragments; Maleimide-PEG₄-Methyltetrazine for Id₂ and Id₄) to enable conjugation overnight at room temperature. Labeled and dual-modified peptides may be purified using HPLC, dialysis or desalting columns to remove excess (and any unreacted) Maleimide-containing reagent, and to exchange the peptides into 1×PBS. Labeled and dual-modified peptides may also be precipitated using a variety of methods known in the art, and resuspended in 1×PBS buffer. FPND Identimers may then be used right away, stored for several days at 4° C. or at −20° C. for longer term storage.

### C. Experiment 1— FPND Identimer-Based Molecular Barcode Validation

Validation of FPND Identimer-based molecular barcode performance may be accomplished using a four-cycle deconvolution/decoding by cleavage experiment to confirm the concatenation of the identimers to form and encode the FPND Identimer-based molecular barcodes and their detectable signal response upon decoding by orthogonally reactive cleavage.

Generally, to detect a detectable signal response, a series of imaging and cleaving steps is performed to decode FPND identimer-based molecular barcodes present on members of the combinatorial library (i.e., a molecular barcode fixed to a bead). This process can be repeated in cycles, whereby the next orthogonal protease can be introduced during each cleavage cycle, followed by an imaging step.

In Experiment 1, TEV protease is introduced in the first cleaving step to decrease observed signal intensity of fluorophore attached only to TEVp-reactive identimers (i.e., Id₅) followed by the first imaging step to visually identify the detectable labels cleaved from Id₅ tokens incorporated in the molecular barcodes comprising the combinatorial library. The cleavage cycles introduce the listed proteases in the following order: TEVp during cycle 1; TVMVp during cycle 2; SuMMVp during cycle 3; and TUMVp during cycle 4.

Id₁ is the first identimer added to the chain, and therefore is directly attached to the solid support (bead); here Id₁ doesn't include a cleavage site and its recognition portion can be an [uncleavable linker]. As outlined above, Id₁ contains the N-terminal TCO modification which can be used for attachment to the solid support (bead); the surface of the solid support is be modified to contain the compatible methyltetrazine moiety for attachment of Id₁. The recognition portions and cleavage sites of Id₂, Id₃, Id₄, and Id₅ are the recognition sequences and cleavage sites of, respectively, TUMVp, SuMMVp, TVMVp, and TEV protease.

As used in Experiment 1, Id₁₋₅ identimers are formed on beads as described herein. The beads are then be exposed in a first cleavage step to decode the molecular barcodes to a solution containing 2 units of TEVp for a 30-60 minute incubation at 30° C. in protease reaction buffer (50 mM Tris-HCl pH 7.5, 0.5 mM EDTA, 1 mM DTT). Following incubation, the beads are imaged in a first imaging step and fluorophores responsive to TEVp are detected. Here, the detectable signal response comprises a reduction of the intensity in the emission wavelength of the detectable labels of FPND identimer tokens dissociated from the molecular barcode during the first cleavage step. Following a wash step, the beads are exposed in a second cleavage step to a solution containing 2 units of TVMVp for a 30-60 minute incubation at 30° C. in protease reaction buffer. Following incubation, the beads are imaged in a second imaging step and fluorophores responsive to TVMVp are detected. Here, the detectable signal response comprises a reduction of the intensity in the emission wavelength of the detectable labels of FPND identimer tokens dissociated from the molecular barcode during the second cleavage step. Following a further wash step, the beads are exposed in a third cleavage step to a solution containing 2 units of SuMMVp for a 30-60 minute incubation at 30° C. in protease reaction buffer. Following incubation, the beads are imaged in a third imaging step and fluorophores responsive to SuMMVp are detected. Here, the detectable signal response comprises a reduction of the intensity in the emission wavelength of the detectable labels of FPND identimer tokens dissociated from the molecular barcode during the third cleavage step. Finally, in a fourth cleavage step the beads are exposed to a solution containing 2 units of TUMVp for a 30-60 minute incubation at 30° C. in protease reaction buffer. Following incubation, the beads are imaged in a fourth imaging step and fluorophores responsive to TUMVp are detected. Here, the detectable signal response comprises a reduction of the intensity in the emission wavelength of the detectable labels of FPND identimer tokens dissociated from the molecular barcode during the cleavage step. After removal of the final cleavable identimer label, the uncleavable label will remain as the strongest signal emitted from the bead, allowing for its identification and potential use for quantification. The sequence of fluorophores for each barcode that was formed during split-pooling and concatenation is determined during visual deconvolution and can be correlated with the bead it was attached to.

### Example 2 Fluorophore/NUCLEASEsite (FNS) Identimer-Based Molecular Barcode

Example 2 illustrates the use of a Fluorophore/NUCLEASEsite (FNS) Identimer-based molecular barcode for in situ labeling of material designed for visual decoding (FIGS. 5, 7, 9).

A set of one or more FNS Identimer classes may be used to encode and form a visual molecular barcode by split-pool ligation to create a combinatorial library of barcodes that do not require DNA sequencing-based decoding. As shown in Example 2, a single barcode may be generated in the same experiment, and in the presence of many different barcode chains being constructed simultaneously. Many endonucleases are known in the art to possess orthogonal reactivity with respect to one another. For example, NotI, XhoI, SpeI, and HindIII possess specificity for dsDNA sequences containing GCGGCCGC, CTCGAG, ACTAGT, and AAGCTT, respectively. Therefore, NotI should not specifically cleave sites recognized by XhoI, SpeI or HindIII with high efficiency. The same is true with respect to each of the nucleases listed, as each should be capable of cleaving (primarily), only their respective substrates in the presence of substrates recognized by the others. This aspect provides orthogonality during identimer-based molecular barcode identification. Each member of the illustrated identimer barcode library contains five classes (Id₁₋₅) consisting of four cleavable Q-Fluorophore/NUCLEASEsite Identimer tokens (Id₂, Id₃, Id₄, and Id₅) as well as one uncleavable identimer token (Id₁).

Experiment 2 is a four-cycle orthogonal nuclease cleavage experiment, confirmed by visual imaging of reactive fluorophores performed to confirm the encoding and decoding of FNS identimer-based molecular barcodes. Experiment 2 comprises a first imaging step to record detectable signal conveyed by the detectable labels making up FNS Identimer-based molecular barcodes present on any member of the library (bead). NotI is introduced in a first cleavage step to decrease observed signal intensity of the fluorophore attached only to the NotI nuclease-reactive identimer, allowing for the visual identification of the label attached to Id₅. This process can be repeated in cycles, whereby the next orthogonal nuclease can be introduced during each cleavage step, followed by an imaging step.

After the first imaging step, each cycle of experiment 2 comprises a cleavage step and an imaging step. Nuclease cleavage agents are applied to the molecular barcode in the following order: NotI during cycle 1; XhoI during cycle 2; SpeI during cycle 3; and HindIII during cycle 4. Imaging after each cycle will enable deconvolution of the order that each visual label making up the FNS Identimer-based molecular barcode was added. Limit of detection, specificity, and precision experiments (in triplicate) of nucleases acting as cleaving agents may be performed to establish the reproducibility of nuclease orthogonal reactivity to the FNS Identimer-based molecular barcode.

In some embodiments, a FNS Identimer scaffold portion includes a ssDNA having a 5′ and 3′ end that is fluorescently labeled with a detectable label through a modified internal base, and is pre-hybridized with a complementary ssDNA to form a dsDNA duplex. The formed dsDNA duplex may encode one or more copies of a single endonuclease restriction sequence (or site type). One having ordinary skill in the art, with the benefit of this disclosure, will understand that efficient dissociation of detectable labels from a formed and encoded molecular barcode may be facilitated by selectively spacing the endonuclease restriction sequences along the scaffold portion. In some embodiments, one strand of the dsDNA duplex containing the nuclease recognition sequence(s) comprises an internal-amino modification for attachment of a pre-designated fluorophore.

As used in Experiment 2, the scaffold portion of each FNS identimer class member comprises the same restriction endonuclease site type, but receives a different and distinguishing fluorophore. The amino-reactive heterobifunctional crosslinking reagent, NHS-PEG₄-TCO, may then be used to chemically modify the installed primary amine on the dsDNA to contain the click-compatible TCO group. The Methyltetrazine-modified fluorophores may then be conjugated with identimer dsDNA duplexes.

As used in Experiment 2, the scaffold portions of FNS identimers comprise unpaired 3′-ends that are compatible for ligation with other FNS Identimer classes to facilitate building a combinatorial chain of identimers by split-pooling. Each round of addition to the growing FNS Identimer molecular barcode will add a unique identimer token to the forming molecular barcode.

As used in Experiment 2, Id₁ is the first identimer added, and therefore is directly attached to the solid support (i.e., bead). Id₁ doesn't include a cleavage site and its recognition portion is an uncleavable linker. Id₁ contains a 5′-amino modified base for attachment to the solid support. As used in Experiment 2, the recognition portions and cleavage sites of Id₂, Id₃, Id₄, and Id₅ are the recognition sequences and cleavage sites of, respectively, HindIII, SpeI, XhoI, and NotI endonucleases.

As used in Experiment 2, the detectable labels of the Id₁₋₅ classes comprise one or more quantum dots. Each FNS identimer class comprises quantum dots having different emission wavelengths that are covalently attached to the FNS identimers in different reaction mixtures (or wells of a plate) for each class-specific dsDNA bearing a specific nuclease site type. As used in Experiment 1, quantum-dot detectable labels are added, respectively, to Id₁, Id₂, Id₃, Id₄, and Id₅ through an internally-modified base located within one or both strands of the dsDNA sequence. The combination of fluorophores displayed on any formed and encoded FNS Identimer-based molecular barcode in the library (Id₁, Id₂, Id₃, Id₄, and Id₅ shown here) may or may not have contiguous emission spectra, as the three-dimensional configuration of identimer tokens comprising the molecular barcodes making up the library is determined by the cycling of orthogonal nucleases as described herein.

### A. FNS Identimer Concatenation

As used in Experiment 2, the FNS Identimer recognition moieties comprise complementary ssDNA strands forming dsDNA duplexes. The FNS identimer cleavage sites comprise specific restriction endonuclease sites included in the dsDNA duplexes and are formed by hybridization, whereby the two strands share significant complementarity, but remain unpaired at their 3′-ends. The unpaired 3′-ends of each FNS Identimer class are designed to be complementary with acceptor 3′-ends on adjacent class members.

In some embodiments, FNS Identimers may be sequentially added in a pre-defined order to form barcodes over sequential rounds of splitting and pooling. Each round of addition will enable concatenation of identimer dsDNA through enzymatic ligation of the 5′- and 3′-ends of the ssDNA fragments of hybridized identimers. The ligation of each identimer may be performed in 1× ligase buffer (50 mM Tris-HCl pH 7.5, 10 mM MgCl₂, 1 mM ATP, 10 mM DTT) in the presence of 500-1,000 U of T4 DNA ligase and 20-80 U of Polynucleotide Kinase over a 25-minute incubation at 37° C. The illustrated Fluorophore/NUCLEASEsite Identimer barcode contains 5 class members joined by enzymatic ligation.

### B. Preparation of FNS Identimers

NotI, XhoI, SpeI, and HindIII are endonucleases possessing specificity for dsDNA sequences containing GCGGCCGC, CTCGAG, ACTAGT, and AAGCTT, respectively. As used in Experiment 2, Id₁ is configured to be uncleavable and the recognition moieties of ID₂₋₅ comprise, respectively: Id₂ comprises ATTTATTTAAGCTTATTA/iAmMC6T/TATTT (SEQ ID NO: 5) containing the HindIII cleavage site; Id₃ comprises ATTTATTTAACTAGTATTA/iAmMC6T/TATTT (SEQ ID NO: 6) containing the SpeI cleavage site; Id₄ comprises ATTTATTTACTCGAGATTA/iAmMC6T/TATTT (SEQ ID NO: 7) containing the XhoI cleavage site; and Id₅ comprises ATTTATTTAGCGGCCGCATTA/iAmMC6T/TATTT (SEQ ID NO: 8) containing the NotI cleavage site. The uncleavable dsDNA of Id₁ may consist of any DNA sequence that is not recognized by any of the nucleases used for recognition of other class members making up formed FNS Identimer-based molecular barcodes.

As used herein, “iAmMC6T” refers to Int Amino Modifier C6 dT, available commercially from Integrated DNA Technologies, Inc. (1710 Commercial Park, Coralville, Iowa 52241, USA). In some embodiments, modified nucleotides (e.g., iAmMC6T) are configured to be far enough away from a recognition moiety so as to not interfere with a cleaving agent's access to a recognition moiety once the modified nucleotide is labeled. In some embodiments, the modified nucleotide is configured to be at least 6 bp away from the recognition moiety. In some embodiments, modified nucleotides placed internally within a looped dsDNA are placed far enough away from each other to avoid FRET-based quenching. In some embodiments, modified nucleotides placed internally within a looped dsDNA are placed far enough away from each other to avoid enzyme recognition sites and facilitate enzyme accessibility.

As used in Experiment 2, each FNS Identimer class is generated by first annealing a complementary strand that together with the class-specific ssDNA strands listed above (Id₂₋₅) to generate the viable dsDNA restriction sites. This is done in a 1:1 molar ratio in NHS-compatible annealing buffer (100 mM Sodium Phosphate buffer, pH 8.5, supplemented with 80 mM KCl) at 95° C. for 5 minutes on a heat block, the heat block is then removed and allowed to cool to room temperature on the benchtop over what amounts to be about a 2-hour time period.

In some embodiments, the identimer class-specific dsDNA bearing the single endonuclease site type may be modified (through the internal-amino modification shown in the sequences above) using NHS-PEG₄-transcyclooctene, such that it contains a compatible chemistry for downstream conjugation. To accomplish this, 100 μM of the labeled dsDNA is mixed with 500 μM NHS-PEG₄-transcyclooctene (TCO) in NHS-compatible annealing buffer (100 mM Sodium Phosphate buffer, pH 8.5, supplemented with 80 mM KCl), and allowed to react at room temperature overnight. TCO-modified dsDNA duplexes may then be purified by desalting to remove excess (and any unreacted) NHS-PEG₄-TCO reagent. Many methyltetrazine-modified fluorophores are commercially available and can be used for labeling of each class-specific identimer in a designated way. To do this, each class specific identimer could be split into different wells, and 100 μM TCO-modified dsDNA of each class could be conjugated to the different methyltetrazine-modified fluorophores used at 200 μM in NHS-compatible annealing buffer. This reaction could proceed for 30 minutes at room temperature. The conjugated FNS Identimer may then be buffer exchanged into storage buffer (50 mM Tris-HCl pH 7.5 supplemented with 100 mM NaCl), and could be used right away or stored for several days at 4° C. or at −20° C. for longer term storage.

### C. FNS Identimer-Based Molecular Barcode Validation

Identification of FNS identimer-based molecular barcodes may be accomplished using a four-cycle deconvolution/decoding by cleavage experiment to confirm the detectable signal response of Id₁₋₅ and confirm the respective sequential concatenation and encoding of the FNS identimer-based molecular barcode.

In some embodiments, molecular barcode libraries may be formed on beads, and the beads may be immobilized on a surface and imaged before and after contact with experimental solutions. Immobilized beads may first be imaged using a standard fluorescence microscope configured with appropriate excitation wavelengths and emission filters to record the combination of detectable signal responses conveyed by the detectable labels making up each molecular barcode in a given field of view or region of interest. All “Hi-Fidelity” versions of the endonucleases listed can function with 100% efficiency in the same buffer (1× CUTSMART buffer available from New England Biolabs, Inc. at 428 Newburyport Turnpike, Rowley, MA 01969, U.S.A.).

As used in Experiment 2, the beads are exposed in a first cleavage step in a solution containing 5 units of NotI-HF endonuclease for a 5-10 minute incubation at 37° C. in CUTSMART buffer (50 mM Potassium Acetate, 20 mM Tris-acetate, 10 mM Magnesium Acetate, 100 μg/ml BSA; buffer pH is 7.9 at 25° C.). Following incubation, the beads may be imaged in a second imaging step to detect detectable signal response conveyed by the fluorophore detectable labels responsive to NotI endonuclease activity. Here, the detectable signal response comprises a reduction of the intensity in the emission wavelength of the detectable labels of FNS identimer tokens dissociated from the molecular barcode during the first cleavage step. Optionally, a washing step may be performed prior to exposing the beads to the second cleavage step.

The second cleavage step comprises exposing the beads to a solution containing 5 units of XhoI-HF for a 5-10 minute incubation at 37° C. in CUTSMART buffer. Following incubation, the beads are imaged in a second imaging step and the detectable signal response conveyed by fluorophore detectable labels responsive to XhoI endonuclease activity is detected. Here, the detectable signal response comprises a reduction of the intensity in the emission wavelength of the detectable labels of FNS identimer tokens dissociated from the molecular barcode during the second cleavage step. Optionally, a washing step may be performed prior to exposing the beads to the third cleavage step.

The third cleavage step comprises exposing the beads to a solution containing 5 units of SpeI-HF for a 5-10 minute incubation at 37° C. in CUTSMART buffer. Following incubation, the beads are imaged in a second imaging step and the detectable signal response conveyed by fluorophore detectable labels responsive to SpeI endonuclease activity is detected. Here, the detectable signal response comprises a reduction of the intensity in the emission wavelength of the detectable labels of FNS identimer tokens dissociated from the molecular barcode during the third cleavage step. Optionally, a washing step may be performed prior to exposing the beads to the fourth cleavage step.

Finally, the fourth cleavage step comprises exposing the beads to a solution containing 5 units of HindIII for a 5-10 minute incubation at 37° C. in CUTSMART buffer. Following incubation, the beads are imaged in a fourth imaging step and the detectable signal response conveyed by fluorophore detectable labels responsive to HindIII endonuclease activity is detected. Here, the detectable signal response comprises a reduction of signal intensity for the emission wavelengths associated with wavelength of the detectable labels of HindIII FNS identimer tokens dissociated from the molecular barcode during the fourth cleavage step.

Optionally, more than one restriction endonuclease may be introduced during the cleavage step of each cycle, but the endonucleases should be introduced under conditions whereby different endonucleases possess significantly different kinetic cleavage rates, and multiple images should be acquired during cleavage.

After removal of the final cleavable identimer label, the uncleavable detectable label will convey the strongest detectable signal emitted from the bead, allowing for its identification. The sequence of fluorophores for each FNS Identimer barcode that was determined during visual deconvolution can be ascribed to each bead attached to it in this way.

### Example 3 Fluorophore/PROTEASEsite/dsDNA (FPD) Identimer-Based Molecular Barcodes

Example 3 illustrates the use of a Fluorophore/PROTEASEsite/dsDNA (FPD) identimer-based molecular barcode for in situ labeling of material designed for decoding both visually as well as in a NGS reaction milieu (FIGS. 6, 8, 10, 11). A set of one or more of FPD identimer classes may be used to encode and form a NGS-compatible visual molecular barcode by split-pool ligation to create a combinatorial library of barcodes that do not require DNA sequencing-based decoding. As shown in Example 3, a single molecular barcode may be generated in the same experiment, and in the presence of many different molecular barcodes chains being formed and encoded simultaneously. As used in Example 3, each molecular barcode in the combinatorial library contains five FPD identimer classes (Id₁₋₅) comprising four cleavable FPD identimer tokens (Id₂, Id₃, Id₄, and Id₅) as well as one uncleavable FPD identimer token (Id₁).

Experiment 3 is a four-cycle orthogonal protease cleavage experiment, confirmed by visual imaging of reactive fluorophores, performed to validate the encoding and decoding of FPD identimer-based molecular barcodes. Experiment 3 comprises a first imaging step to record detectable signal conveyed by the detectable labels of FNS Identimer-based molecular barcodes present on any member of the library (bead). TEV protease are introduced in the first cycle to decrease the signal intensity of the fluorophore detectable labels of TEV protease-reactive FPD identimer tokens incorporated into a molecular barcode, allowing for the visual identification of the label cleaved from Id₅. This process is repeated in cycles, whereby the next orthogonal protease is introduced during each cleavage cycle, followed by an imaging step.

As used in Experiment 3, the protease cleaving agents are introduced during the cleavage steps in the following order: TEVp during cycle 1; TVMVp during cycle 2; SuMMVp during cycle 3; and TUMVp during cycle 4. Imaging after each cycle will enable deconvolution of the three-dimensional arrangement of the detectable labels of FPD identimer tokens incorporated into FPD identimer-based molecular barcodes. Limit of detection, specificity, and precision experiments (in triplicate) of proteases acting as cleaving agents may be performed to establish the reproducibility of protease orthogonal reactivity to the Fluorophore/PROTEASEsite/dsDNA Identimer-based molecular barcode.

### A. FPD Identimer Scaffold Portions

In some embodiments, a FPD identimer scaffold portion comprises a polypeptide fragment covalently linked to a single-stranded DNA (ssDNA) fragment having a 3′ and 5′ end through an internally amino-modified base in the ssDNA, using an amino-reactive heterobifunctional crosslinking reagent (NHS-PEG₄-methyltetrazine). The polypeptide fragment containing the peptide recognition sequence may be modified to contain an N-terminal lysine residue, and can be subsequently modified with a chemical group compatible for attachment to the modified ssDNA using a different amino-reactive heterobifunctional crosslinking reagent (NHS-PEG₄-transcyclooctene). Following conjugation of the peptide fragment to the modified ssDNA oligonucleotide, a specific and complementary ssDNA oligonucleotide (harboring the same information as the ssDNA oligonucleotide attached to the peptide fragment but in reverse-complement orientation) may be hybridized to the ssDNA of the scaffold portion to generate a dsDNA species with unpaired 3′-ends that will be compatible for ligation with other FPD identimer classes to build a combinatorial chain of identimers by split-pooling. Each round of addition to the FPD identimer-based molecular barcodes will add a unique identimer token to the forming molecular barcodes. Attached to the opposing end of the peptide fragment (C-terminus) is a detectable label (fluorophore) as described below. The emission wavelength (observed color) of the detectable label attached to each FPD identimer is correlated with the sequence of the attached dsDNA, thereby coupling information that may be obtained from the visual barcode with information that can be obtained from the formed NGS barcode after DNA sequencing.

As used in Experiment 3, Id₁ is the first identimer added to the chain, and therefore is directly attached to the solid support (bead); here Id₁ doesn't include a cleavage site and its recognition portion may be an uncleavable linker. Id₁ comprises a 5′-amino modified base for attachment to the solid support, and may encode a 5′-constant region for retrieval and downstream NGS library preparation. Id₅ comprises a 3′-capture sequence for the capture of macromolecules from cell lysate or a biological sample. The recognition portions and cleavage sites of Id₂, Id₃, Id₄, and Id₅ are the recognition sequences and cleavage sites of, respectively, TUMVp, SuMMVp, TVMVp, and TEV protease.

### B. FPD Identimer Detectable Labels

As used in Experiment 3, quantum dot fluorophore detectable labels comprising multiple distinguishable detectable labels are selected for combinatorial labeling of the FPD identimers in Example 3. Detectable labels conveying different emission wavelengths of detectable signal may be covalently attached to each FPD identimer class, in different reaction mixtures (or wells of a plate) for each class-specific peptide.

In some embodiments, detectable labels may be added respectively, to Id₁, Id₂, Id₃, Id₄, and Id₅ at the opposing end of their respective scaffold portions (C-terminal end) from that which the dsDNA portion of the identimer is conjugated. The combination of fluorophores displayed on any formed FPD identimer-based barcode in the combinatorial library (Id₁, Id₂, Id₃, Id₄, and Id₅ shown here) may or may not convey detectable signal having a contiguous emission spectra, as the three-dimensional arrangement of the detectable labels of FPD identimer tokens incorporated into molecular barcodes making up the combinatorial library is determined by cycling of orthogonal proteases as described above.

### C. FPD Identimer Concatenation

The sequence of each ssDNA fragment is designed to record the emission wavelength of the attached detectable label. The complementary ssDNA strands making up each FPD Identimer scaffold portion are formed by hybridization, whereby the two strands share significant complementarity, but remain unpaired at their 3′-ends. The unpaired 3′-ends of each FPD identimer class are designed to be complementary with acceptor 3′-ends on adjacent FPD identimer tokens incorporated into a molecular barcode. FPD identimers can be sequentially added in a pre-defined order to form molecular barcodes over sequential rounds of splitting and pooling. Each round of FPD identimer addition will enable concatenation of FPD identimer dsDNA through enzymatic ligation of the 5′- and 3′-ends of the ssDNA fragments of hybridized identimers. The ligation of each identimer may be performed in 1× ligase buffer (50 mM Tris-HCl pH 7.5, 10 mM MgCl₂, 1 mM ATP, 10 mM DTT) in the presence of 500-1,000 U of T4 DNA ligase and 20-80 U of Polynucleotide Kinase over a 25-minute incubation at 37° C. The dsDNA of the final “capping” FPD identimer token may be designed to encode both the wavelength of the detectable label of a FPD identimer as well as a 3′ capture region designed to capture macromolecules from cell lysate. A unique molecular identifier (UMI) may also be included as a contiguous stretch of randomized or semi-randomized bases within a capping FPD identimer or may be added to the NGS barcode with each identimer class in smaller stretches of randomized or semi-randomized bases.

### D. Preparation of FPD Identimers

Identimer class-specific oligopeptides designed to comprise a single protease recognition site may be purchased from a commercial vendor.

As used in Experiment 3, Id₁ is configured to not be cleavable and the recognition portions of Id₂₋₅ comprise peptide sequences including, respectively: Id₂ comprising KGGSGGGSACVYHQSGGSGGSC (SEQ ID NO: 9) containing the TUMV cleavage site; Id₃ comprising KGGSGGGSEEIHLQSGGSGGSC (SEQ ID NO: 10) containing the SuMMV cleavage site; Id₄ comprising KGGSGGGSETVRFQGGGSGGSC (SEQ ID NO: 11) containing the TVMV cleavage site; and, Id₅ comprising KGGSGGGSENLYFQSGGSGGSC (SEQ ID NO: 12) containing the TEV cleavage site.

As used in Experiment 3, each FPD identimer class is generated by first modifying the peptide (through a lysine residue installed at the N-terminus of the peptide) using NHS-PEG₄-transcyclooctene, such that it contains a compatible chemistry for downstream conjugation. To accomplish this, 100 μM peptide is mixed with 500 μM NHS-PEG₄-transcyclooctene (TCO) in NHS peptide reaction buffer (100 mM Sodium Phosphate, pH 8.5, supplemented with 80 mM KCl and 70 mM NaCl), and allowed to react at room temperature overnight. TCO-modified peptides are purified using HPLC to remove excess (and any unreacted) NHS-PEG₄-TCO reagent. 100 μM oligonucleotides containing an internal amino-modification and bearing a 5′-phosphate group are modified in NHS-compatible annealing buffer (100 mM Sodium Phosphate buffer, pH 8.5, supplemented with 80 mM KCl) by incubation with 500 μM NHS-PEG₄-methyltetrazine overnight at room temperature. Methyltetrazine-modified oligonucleotides are then buffer-exchanged into NHS-compatible annealing buffer twice, using 7K MWCO Zeba desalting columns to remove excess and any unreacted NHS-PEG₄-methyltetrazine reagent. TCO-modified peptides of each FPD identimer class are conjugated to the different tetrazine-modified ssDNA oligonucleotides (each containing a different nucleotide sequence that corresponds to the detectable labels conjugated to the identimer) by mixing in a 1:1 ratio at 25 μM each. This reaction is allowed to proceed for 30 minutes at room temperature. Complementary ssDNA oligonucleotides are annealed to form dsDNA by incubation with conjugates in a 1:1 molar ratio in NHS-compatible annealing buffer (100 mM Sodium Phosphate buffer, pH 8.5, supplemented with 80 mM KCl) at 95° C. for 5 minutes on a heat block. The heat block is then removed and allowed to cool to room temperature on the benchtop over what amounts to be about a 2-hour time period. Id₁ class members receive a complementary oligo strand containing a 5′-amino modification for chemical modification and subsequent attachment to the solid support. All complementary ssDNA oligonucleotides contain a 5′-phosphate modification for competent ligation to adjacent identimers during barcode formation. The annealed identimers are then labeled with maleimide-modified fluorophores via the installed cysteine residue at their C-terminal ends, by addition of each fluorophore to each FPD identimer class (each FPD identimer class receives one designated fluorophore, that is recorded within the attached dsDNA). FPD identimers are then buffer exchanged into storage buffer (50 mM Tris-HCl pH 7.5 supplemented with 100 mM NaCl), and can be used right away, stored for several days at 4° C. or at −20° C. for longer term storage.

### E. FPD Identimer-Based Molecular Barcode Validation

Identification of FPD Identimer molecular barcodes may be accomplished using a four-cycle deconvolution/decoding by cleavage experiment to confirm the detectable signal response and confirm the respective forming and encoding of the FPD identimer molecular barcode. In some embodiments, FDP identimer-based molecular barcode libraries may be formed on beads, and the beads may be immobilized on a surface and imaged before and after contact with experimental solutions. Immobilized beads would first be imaged using a standard fluorescence microscope configured with appropriate excitation wavelengths and emission filters to record the combination of visual detectable labels making up each barcode in a given field of view or region of interest.

As used in Experiment 3, the first cleavage step comprises exposing the beads in a first cleaving step to a solution containing 2 units of TEVp for a 30-60 minute incubation at 30° C. in protease reaction buffer (50 mM Tris-HCl pH 7.5, 0.5 mM EDTA, 1 mM DTT). Following incubation, the beads are imaged in a first imaging step and fluorophore detectable labels conveying detectable signal response to TEVp are detected. Here, the detectable signal response comprises a reduction of the intensity in the emission wavelength of the detectable labels of FPD identimer tokens dissociated from the molecular barcode during the first cleavage step. Optionally, a washing step may be performed prior to the second cleavage step.

As used in Experiment 3, the second cleavage step comprises exposing the beads to a solution containing 2 units of TVMVp for a 30-60 minute incubation at 30° C. in protease reaction buffer. Following incubation, the beads are imaged in a second imaging step and fluorophore detectable labels responsive to TVMVp are detected. Here, the detectable signal response comprises a reduction of the intensity in the emission wavelength of the detectable labels of FPD identimer tokens dissociated from the molecular barcode during the second cleavage step. Optionally, a washing step may be performed prior to the third cleavage step.

As used in Experiment 3, the third cleavage step comprises exposing the beads to a solution containing 2 units of SuMMVp for a 30-60 minute incubation at 30° C. in protease reaction buffer. Following incubation, the beads are imaged in a second imaging step and fluorophore detectable labels conveying a detectable signal response to SuMMVp are detected. Here, the detectable signal response comprises a reduction of the intensity in the emission wavelength of the detectable labels of FPD identimer tokens dissociated from the molecular barcode during the third cleavage step. Optionally, a washing step may be performed prior to the third cleavage step.

As used in Experiment 3, the fourth cleavage step comprises exposing the beads to a solution containing 2 units of TUMVp for a 30-60 minute incubation at 30° C. in protease reaction buffer. Following incubation, the beads are imaged in a fourth imaging step and fluorophore detectable labels conveying a detectable signal response to TUMVp are detected. Here, the detectable signal response comprises a reduction of the intensity in the emission wavelength of the detectable labels of FPD identimer tokens dissociated from the molecular barcode during the fourth cleavage step.

After removal by cleavage of the final detectable labels from cleavable identimers, the uncleavable label will remain as the strongest detectable signal conveyed from the bead, allowing for its identification. The sequence of fluorophores for each molecular barcode that was determined during visual deconvolution is represented in the sequence of the NGS barcode, which can be retrieved and correlated with the bead following a NGS experiment.

### F. Example 3 Notes

In some embodiments, the recognition portion of every FPD identimer class comprises essentially identical protease recognition sites.

In some embodiments, an identimer class size is based on how many unique detectable labels are available. With Q-dots, this number is about 10, while with standard fluorophores this number is about four to about six. Dual labeling makes the class size 100 because each class member may be labeled with two Q-dots. This can also be done using combinations of standard fluorophores.

In some embodiments, the structural portion of a set of one or more FPD identimers is constructed by first conjugating different ssDNA strands to the same peptide (it has the same protease recognition site, and the same conjugation chemistry in all cases) through the N-terminal end of the peptide and an internal nucleotide modification sitting in the middle of the DNA sequence. That can be done in different tubes or wells (for example wells 1-10). Then (with the structural portions of the FPD identimers still separated in the 1-10 different tubes/wells), the structural portions are washed or otherwise purified, and only one Q-dot type is attached to each of the structural portions and thereby constitute a FPD identimer class (by the C-terminal ends of the peptides). FPD identimer class construction may be performed in different reactions to generate further FPD identimer classes to include in the set of FPD identimers. This is designed so that we know which ssDNA goes with which Q-dot. Then (with the FPD identimer classes still separated in the 1-10 different tubes/wells), the FPD identimer classes are washed or otherwise purified, and a complementary ssDNA is hybridized to each identimer in the set of FPD identimers (the complementary ssDNA being configured to be complementary to the ssDNA of the structural portions of identimers in the set of FPD identimers) to form a dsDNA with 3′ unpaired ends. One having ordinary skill in the art will understand that the complementary ssDNA will not ligate to the wrong FPD identimer class, thus providing a user a degree of control over how to configure and construct the FPD identimer classes. In some embodiments, a user may configure a FPD identimer class to have a reaction specificity in which, for example, a Class B will ligate essentially only with class A and C; Class 3 will essentially only ligate with Blass B and D and so forth. In some embodiments, construction of a FPD identimer comprises first conjugating the peptide to the Q-dot prior to adding a pre-hybridized dsDNA segment to complete the structural portion and form the FPD identimer.

In some embodiments, a FPD identimer construction process may be repeated for each FPD identimer class (each peptide that contains a different protease recognition site) and the FPD identimer classes may then be arranged for storage in tubes, or in a plate or plates depending on the size of each class and the number of classes. For example, as shown in Example 3, five FPD identimer classes comprising ten identimers each may put in 50 different wells of a plate. Thus, upon making a combinatorial library comprising beads and a set of one or more molecular barcodes, the beads will have a first FPD identimer class attached to them in 10 different wells. The beads may then be washed, pooled, and randomly split into the next 10 wells for addition of a second FPD identimer class providing coverage of the combinatorial diversity of the library (all possible combination of what is now 100 different members, represented across 2 classes). Then the above process is repeated; the beads may be washed, pooled, and randomly split into the next 10 wells for addition of a third FPD identimer class, resulting in 1,000 members of the library represented across 3 classes.

In some embodiments, the FPD identimer construction process repeats until a total of 5 FPD identimer classes have been added—constituting a 1M member combinatorial library. Importantly, the final “capping” FPD identimer class in Example 3 comprises a 3′-capture region for the capture of macromolecules from a reaction mixture (cell lysate for example). This 3′-capture region may be a poly(T) tag for capture of polyadenylated RNAs, or a specific tag for capture of nucleotide-tagged macromolecules used as reporters in an assay.

In terms of decoding formed and encoded molecular barcodes comprising a combinatorial library, prior to a first cleavage step, the emission spectra of the detectable signals conveyed from each identimer token incorporated into the molecular barcode will appear in combination, with multiple overlapping spectra (similar to a rainbow). Thus, prior to a first cleavage step, the three-dimensional arrangement of FPD identimer tokens incorporated into a molecular barcode is not decodable. However, knowing the order in which FPD identimer classes were added to the combinatorial library the three-dimensional arrangement allows for decoding the molecular barcode by cycling the proteases through the cleavage steps one protease at a time and in a known order because each cleaving agent protease will essentially cleave only the detectable labels of FPD identimer tokens derived from FPD identimer classes comprising a recognition portions having orthogonal reactivity to the cleaving agent.

In other words, molecular barcode decoding comprises detecting which detectable signal response (in this embodiment colors) are conveyed during exposure to a corresponding protease. In some embodiments, a rainbow-colored bead will be missing an entire color, or have significantly less intensity for a given color, when we image after exposure to the first protease, then we would wash and add the next protease and a second color will be missing or have significantly less intensity when we image.

### Example 4 Fluorophore/NUCLEASEsite/dsDNA(FND) Identimer-Based Molecular Barcodes

Example 4 illustrates the use of a Fluorophore/NUCLEASEsite/dsDNA (FND) identimer-based molecular barcode for in situ labeling of material designed for decoding both visually as well as in a next generation sequencing (NGS) reaction milieu (FIGS. 5, 7, 8, 9, 11). A series of FND identimer classes may be used to encode and form a NGS-compatible visual molecular barcode by split-pool ligation to create a combinatorial library of barcodes that do not require DNA sequencing-based decoding. As shown in Example 4, a single molecular barcode may be generated in the same experiment and in the presence of many different molecular barcodes being formed and encoded simultaneously.

One having ordinary skill in the art will understand that many endonucleases possess orthogonal reactivity with respect to one another. For example, NotI, XhoI, SpeI, and HindIII possess specificity for dsDNA sequences containing GCGGCCGC, CTCGAG, ACTAGT, and AAGCTT, respectively. Thus, NotI should not specifically cleave sites recognized by XhoI, SpeI or HindIII with high efficiency. In other words, each endonuclease should be capable of cleaving a molecular barcode at the cleavage sites of identimers having recognition moieties comprising the respective endonuclease's cleavage site sequence in the presence of substrates recognized by the other endonucleases, thus facilitating the orthogonal reactivity of the endonuclease cleaving agent during FND identimer-based molecular barcode decoding.

As used in Example 4, a combinatorial library comprising beads and a set of one or more FND identimer-based molecular barcode includes five FND identimer classes (Id₁₋₅) with Id₁ being configured to be an uncleavable identimer class and Id₂₋₄ being configured to be cleavable identimer classes. Experiment 4 is a four-cycle orthogonal nuclease cleavage experiment, confirmed by visual imaging of reactive fluorophores, performed to validate the encoding and decoding of FND identimer-based molecular barcodes on the beads.

Experiment 4 comprises four cycles, each cycle comprising one or more cleavage steps followed by one or more imaging steps. Orthogonal cleaving agent nucleases may be applied one at a time during a cleavage step to the beads of the combinatorial library to facilitate orthogonal cleavage of formed and encoded molecular barcodes on the beads. A preliminary imaging step may be performed to detect detectable signal response conveyed from the detectable labels of FND identimer tokens incorporated into a molecular barcode present on any of the beads.

As illustrated in Experiment 4, NotI is introduced in the first cycle during a first cleavage step to decrease the intensity of detectable signal response convey by the detectable labels of FND identimer tokens reactive to NotI nuclease (in this case Id₅ tokens) allowing for the decoding or visual identification of the detectable labels dissociated from the Id₅ tokens through cleavage. As illustrated in Experiment 4, orthogonally reactive cleaving agent nucleases are introduced during one or more of the cleavage steps in the following order: NotI during cycle 1; XhoI during cycle 2; SpeI during cycle 3; and Hindi II during cycle 4. Detection of detectable signal response conveyed from dissociated detectable labels during one or more of the imaging steps facilitates decoding the three-dimensional arrangement of FND identimer tokens incorporated into the molecular barcodes. Limit of detection, specificity, and precision experiments (in triplicate) of nucleases acting as cleaving agents may be performed to establish the reproducibility of nuclease orthogonal reactivity to the FND Identimer-based molecular barcodes.

### A. FND Identimer Scaffold Composition

In some embodiments, a FND identimer scaffold portion comprises a pre-hybridized double-stranded DNA (dsDNA) having one or more copies of a single endonuclease restriction sequence. One strand of the pre-hybridized dsDNA having the endonuclease recognition sequence(s) bears a 5′-amino modification. The amino-reactive heterobifunctional crosslinking reagent, NHS-PEG₄-TCO, may then be used to chemically modify the primary amine on the pre-hybridized dsDNA to contain the click-compatible TCO group. The pre-hybridized dsDNA is covalently linked to a single-stranded DNA (ssDNA) fragment having a 3′ and 5′ end through an internally amino-modified base in the ssDNA, that can be converted to the click-compatible methyltetrazine group using an amino-reactive heterobifunctional crosslinking reagent (NHS-PEG₄-methyltetrazine). Following conjugation of the pre-hybridized dsDNA fragment to the modified ssDNA oligo, a specific and complementary ssDNA oligonucleotide (harboring the same information as the ssDNA oligonucleotide is attached to the dsDNA fragment but in reverse-complement orientation) and may be hybridized to the ssDNA of the identimer to generate a dsDNA species with unpaired 3′-ends that will be compatible for ligation with other FND identimer classes to facilitate building a combinatorial chain of identimers by split-pooling.

In some embodiments, a labeled ssDNA strand comprising at least one detectable label (in this case, a fluorophore) is annealed to the attached ssDNA strand containing the specific endonuclease site. In some embodiments the labeled ssDNA strand is dually labeled on its opposing 5′- and 3′-ends to include four detectable labels. In some embodiments, the dually labeled ssDNA comprises four different detectable labels, each detectable label having a unique detectable signal response, to provide 16 FND identimer classes. In some embodiments, the detectable labels are operatively connected to the scaffold portion at a spaced-apart distance relative to each other to reduce fluorescent quenching or other non-specific reactions between the detectable labels.

As shown in FIG. 14, the labeled ssDNA strand is dual-labeled on its opposing 5′- and 3′-ends, and the sequence of the associated dsDNA duplex contains two restriction sites of the same type. The emission wavelength (observed color) of the detectable labels of each FND identimer is correlated with the sequence of the attached dsDNA (that is competent for ligation), thereby coupling information that can be obtained from the molecular barcode with information that can be obtained from the formed NGS barcode after DNA sequencing. To avoid cross-reactivity, the designed sequences making up the formed NGS barcode should not contain sequences that may be cleaved by any of the restriction enzymes used. Id₁ is the first identimer added and is therefore directly attached to solid support (bead). Here Id₁ doesn't include a cleavage site and its recognition portion is an uncleavable linker. Id₁ comprises a 5′-amino modified base for attachment to the solid support, and can encode a 5′-constant region for retrieval and downstream NGS library preparation. The structural portions of Id₅ contains a 3′-capture sequence for the capture of macromolecules from cell lysate or a biological sample. The recognition portions and cleavage sites of Id₂, Id₃, Id₄, and Id₅ are the recognition sequences and cleavage sites of, respectively, HindIII, SpeI, XhoI, and NotI endonucleases.

### B. FND Identimer Detectable Labels

Commercially available fluorophores comprising multiple distinguishable labels are selected for combinatorial labeling of the FND identimers in this example. In a preferred embodiment, detectable labels of the FND identimers comprise one or more Q-dots. In some embodiments, fluorophores of different emission wavelengths may be covalently attached to each identimer class, in different reaction mixtures (or wells of a plate) for each class-specific dsDNA bearing a specific nuclease site type. A single molecular barcode made up of five of FND identimer class types is illustrated in Example 4. Fluorophores may be added respectively, to Id₁, Id₂, Id₃, Id₄, and Id₅ at either end (3′-end shown) of the ssDNA strand annealed to the strand attached to the ligation competent dsDNA segment making up the NGS portion of the identimer barcode. The combination of fluorophores displayed on any formed individual FND identimer-based molecular barcode in the combinatorial library (Id₁, Id₂, Id₃, Id₄, and Id₅ shown here) may or may not have contiguous emission spectra, as the three-dimensional arrangement of FND identimer tokens incorporated into the molecular barcodes is determined by the cycling of cleavage steps using orthogonally reactive nucleases described herein.

### C. FND Identimer Concatenation

The sequence of each ssDNA fragment is designed to record the emission wavelength a detectable signal response conveyed from dissociated detectable labels of FND identimer tokens incorporated into a molecular barcode. The complementary ssDNA strands of each FND identimer are formed by hybridization, whereby the two strands share significant complementarity, but remain unpaired at their 3′-ends. In some embodiments, the unpaired 3′-ends of concatenated FND identimer tokens are configured to be complementary with acceptor 3′-ends of adjacent FND identimer tokens. FND identimers may be sequentially added in a pre-defined order to form molecular barcodes over sequential rounds of splitting and pooling. Each round of FND identimer class addition will enable concatenation of identimer dsDNA through enzymatic ligation of the 5′- and 3′-ends of the ssDNA fragments of hybridized identimers. The ligation of each identimer may be performed in 1× ligase buffer (50 mM Tris-HCl pH 7.5, 10 mM MgCl₂, 1 mM ATP, 10 mM DTT) in the presence of 500-1,000 U of T4 DNA ligase and 20-80 U of Polynucleotide Kinase over a 25-minute incubation at 37° C. In some embodiments, a final “capping” FND identimer class comprises dsDNA (making up the NGS portion of the barcode) to encode both the wavelength of the label attached to the identimer polypeptide protease recognition portion, as well as a 3′ capture region designed to capture macromolecules from cell lysate. A unique molecular identifier (UMI) may also be included as a contiguous stretch of randomized or semi-randomized bases within the capping FND identimer class, or may be added to the NGS barcode with each identimer class in smaller stretches of randomized or semi-randomized bases. To avoid cross-reactivity, the configured sequences making up the formed NGS barcode, including those making up the UMI, essentially do not contain sequences that can be cleaved by any of the restriction enzymes used. In some embodiments, computational filtering of configured molecular barcodes may be used to avoid the inclusion of one of the restriction sites used for the decoding by cleavage experiment. The FND identimer barcode contains 5 class members joined by enzymatic ligation.

### D. FND Identimer Class Preparation

NotI, XhoI, SpeI, and HindIII possess specificity for dsDNA sequences containing GCGGCCGC, CTCGAG, ACTAGT, and AAGCTT, respectively. Each FND identimer class-specific ssDNA making up the labeled dsDNA containing one or more restriction endonuclease site types may be purchased from a commercial vendor. As used in Experiment 4, the recognition portions and cleavage sites of the FND identimer classes comprise nucleotide sequences configured, respectively: (Id₁ is not cleavable.); Id₂ comprising/5AmMC6/ATTTATTTAAGCTTATTATTATTT (SEQ ID NO: 13) containing the HindIII cleavage site; Id₃ comprising/5AmMC6/ATTTATTTAACTAGTATTATTATTT (SEQ ID NO: 14) containing the SpeI cleavage site; Id₄ comprising/5AmMC6/ATTTATTTACTCGAGATTATTATTT (SEQ ID NO: 15) containing the XhoI cleavage site; and Id₅, comprising/5AmMC6/ATTTATTTAGCGGCCGCATTATTATTT (SEQ ID NO: 16) containing the NotI cleavage site.

In some embodiments, amino modifications are only on the 5′ end of one of the oligonucleotides making up an FND Identimer scaffold portion that is attached to the bead. Thus, all other dsDNA FND Identimer scaffold portion contain internal amino modifications so that the ends are available for ligation of flanking FND Identimer scaffold portions. For example, in some embodiments the amino modifications are on 5′ ends of all hybridizing FND Identimers, because for each hybridizing duplex, one strand's 5′ end is biotinylated and the other strand of the duplex contains a label attached via NHS-modified fluorophores. In some embodiments, the amino modifications are included within internal positions of the scaffold portions of all identimers in a set of ringed or circular dsDNA-based identimers so that free ends will be available for ligation.

As used herein, “5AmMC6” refers to Amino Modifier C6. Skilled persons will understand that amino modifiers are used to introduce a primary amino group into an oligonucleotide. For example, an amino modifier may be used in conjunction with a NHS ester or isothiocyanate fluorescent detectable labels. Skilled persons will understand that Amino Modifier C6 may be incorporated during oligonucleotide synthesis and can be used to label the 5′ end of an oligonucleotide with a primary amino group at the end of a six-carbon spacer. As used herein, “N-Hydroxysuccinimide” (NHS) refers to an organic compound with the formula (CH₂CO)₂NOH. Skilled persons will understand that N-hydroxysuccinimide esters or “NHS-esters” may be used to site-specifically modify primary amine groups installed within synthesized oligonucleotides that contain such groups at designed locations, similarly to the way proteins may be non-selectively on free amino groups by ester-mediated derivatization (see e.g., Nanda et al., Methods Enzymol., 536:87-94 (2014)). For example, skilled persons will understand that NHS-esters may be used to label to the primary amines (R—NH₂) or proteins, amine-modified nucleotides, and other amine-containing molecules. Thus, as used herein, “NHS fluorophore” refers to any fluorophore conjugated to NHS.

In some embodiments, FND identimer class may be generated by first annealing a complementary, labeled strand that together with the class-specific ssDNA strands listed above (Id₂₋₅), generate the viable dsDNA restriction sites. This may be done in a 1:1 molar ratio in NHS-compatible annealing buffer (100 mM Sodium Phosphate buffer, pH 8.5, supplemented with 80 mM KCl) at 95° C. for 5 minutes on a heat block, the heat block is then removed and allowed to cool to room temperature on the benchtop over what amounts to be about a 2-hour time period. The class-specific dsDNA bearing the single endonuclease site type may be modified (through the 5′-amino modification shown in the sequences above) using NHS-PEG₄-transcyclooctene, such that it contains a compatible chemistry for downstream conjugation. To accomplish this, 100 μM of the labeled dsDNA may be mixed with 500 μM NHS-PEG₄-transcyclooctene (TCO) in NHS-compatible annealing buffer (100 mM Sodium Phosphate buffer, pH 8.5, supplemented with 80 mM KCl), and allowed to react at room temperature overnight. TCO-modified dsDNA duplexes may be purified by desalting to remove excess (and any unreacted) NHS-PEG₄-TCO reagent. 100 μM oligonucleotides containing an internal amino-modification are first annealed to complementary ssDNA to form dsDNA encoding the NGS portion of the barcode by incubation in a 1:1 molar ratio in NHS-compatible annealing buffer (100 mM Sodium Phosphate buffer, pH 8.5, supplemented with 80 mM KCl) at 95° C. for 5 minutes on a heat block, the heat block is then removed and allowed to cool to room temperature on the benchtop over what amounts to be about a 2-hour time period.

As used in Experiment 4, Id₁ class NGS barcode portions receive a complementary oligo strand containing a 5′-amino modification for chemical modification and subsequent attachment to the solid support. All complementary ssDNA oligonucleotides comprise a 5′-phosphate modification for competent ligation to adjacent identimers during barcode formation. The internal amino-modified oligos also bearing 5′-phosphate groups are modified in NHS-compatible annealing buffer by incubation with 500 μM NHS-PEG₄-methyltetrazine overnight at room temperature. Methyltetrazine-modified oligonucleotides are then buffer-exchanged into NHS-compatible annealing buffer twice, using 7K MWCO Zeba desalting columns to remove excess and any unreacted NHS-PEG₄-methyltetrazine reagent. TCO-modified dsDNA of each class are conjugated to the different methyltetrazine-modified ssDNA oligonucleotides (each containing a different nucleotide sequence that corresponds to the detectable label already conjugated to the identimer) by mixing in a 1:1 ratio at 25 μM each. This reaction may be allowed to proceed for 30 minutes at room temperature. The conjugated FND Identimers may then be buffer exchanged into storage buffer (50 mM Tris-HCl pH 7.5 supplemented with 100 mM NaCl), and may be used right away, stored for several days at 4° C. or at −20° C. for longer term storage.

### E. FND Identimer-Base Molecular Barcode Validation

Identification of FND identimer-based molecular barcodes may be accomplished using a four-cycle deconvolution/decoding by cleavage experiment to confirm the detectable signal response conveyed from detectable labels dissociated by cleavage from a molecular barcode to decode its encoded three-dimensional arrangement. In some embodiments, molecular barcode combinatorial libraries may be formed on beads, and the beads may be immobilized on a surface and imaged before and after contact with experimental solutions. Immobilized beads would first be imaged using a standard fluorescence microscope configured with appropriate excitation wavelengths and emission filters to record the combination of visual detectable labels making up each barcode in a given field of view or region of interest. Generally, “Hi-Fidelity” versions of the endonucleases listed herein can function with 100% efficiency in the same buffer (1× CUTSMART buffer available from New England Biolabs, Inc. at 428 Newburyport Turnpike, Rowley, MA 01969, USA).

As used in Experiment 4, the beads are exposed in a first cleavage step to a solution containing 5 units of NotI-HF endonuclease for a 5-10 minute incubation at 37° C. in CUTSMART buffer (50 mM Potassium Acetate, 20 mM Tris-acetate, 10 mM Magnesium Acetate, 100 μg/ml BSA; buffer pH is 7.9 at 25° C.). Following incubation, the beads are imaged in a first imaging step and detectable labels responsive to NotI endonuclease activity are detected. Here, the detectable signal response comprises a reduction of the intensity in the emission wavelength of the detectable labels of FND identimer tokens dissociated from the molecular barcode during the first cleavage step. Optionally, a washing step may be performed prior to the second cleavage step.

As used in Experiment 4, the second cleavage step comprises exposing the beads to a solution containing 5 units of XhoI-HF for a 5-10 minute incubation at 37° C. in CUTSMART buffer. Following incubation, the beads are imaged in a second imaging step and fluorophore detectable labels responsive to XhoI endonuclease activity are detected. Here, the detectable signal response comprises a reduction of the intensity in the emission wavelength of the detectable labels of FND identimer tokens dissociated from the molecular barcode during the second cleavage step. Optionally, a washing step may be performed prior to the third cleavage step.

As used in Experiment 4, the third cleavage step comprises exposing the beads to a solution containing 5 units of SpeI-HF for a 5-10 minute incubation at 37° C. in CUTSMART buffer. Following incubation, the beads are imaged in a third imaging step and fluorophores responsive to SpeI endonuclease activity are detected. Here, the detectable signal response comprises a reduction of the intensity in the emission wavelength of the detectable labels of FND identimer tokens dissociated from the molecular barcode during the third cleavage step. Optionally, a washing step may be performed prior to the fourth cleavage step.

As used in Experiment 4, the fourth cleavage step comprises exposing the beads to a solution containing 5 units of HindIII for a 5-10 minute incubation at 37° C. in CUTSMART buffer. Following incubation, the beads can be imaged in a fourth imaging step and fluorophores responsive to HindIII endonuclease activity are detected. Here, the detectable signal response comprises a reduction of the intensity in the emission wavelength of the detectable labels of FND identimer tokens dissociated from the molecular barcode during the fourth cleavage step.

In some embodiments, more than one restriction endonuclease may be introduced in each cycle of decoding by cleavage, but these must be introduced under conditions whereby different endonucleases possess significantly different kinetic cleavage rates, and multiple images should be acquired during cleavage.

After removal of the final cleavable identimer label, the uncleavable label will remain as the strongest signal emitted from the bead, allowing for its detection and identification. The sequence of fluorophores for each barcode that was determined during visual deconvolution is represented in the sequence of the NGS barcode, which can be retrieved and correlated with the bead following a NGS experiment.

### Example 5 Molecular Barcodes Produced by Split-Pool Ligation of Single-Label and Dual-Label FND Identimers

As disclosed in Example 5, single-label and dual-label Fluorophore/NUCLEASEsite/dsDNA (FND) identimer classes were produced to encode and form molecular barcodes by split-pool ligation to create molecular barcode combinatorial libraries. Multi-cycle orthogonal nuclease cleavage experiments, confirmed by visual imaging of reactive-fluorophore detectable labels, were performed to validate the encoding and decoding of FND identimer-based molecular barcodes. Known cleavage agents were applied to the nuclease cleavage experiments in a known order to facilitate deconvolution of the single-label and dual-label FND identimer classes. The images were then analyzed in reverse chronological order to observe pseudo “signal gain” versus real signal loss.

Single-label and dual-label FND identimer-based molecular barcode combinatorial libraries were generated from a set of five FND identimer classes (FND Id₁₋₅). A set of five NHS-modified fluorophores were conjugated directly to amino-modified bases as described herein. A single-label FND identimer class comprises a recognition moiety that correlates to a single label upon cleavage, whereas a dual-label FND identimer class comprises a recognition moiety that correlates to two labels upon cleavage. For example, a combinatorial library built with an unmixed volume of AF-750 labeled, HindIII-based FND identimers will lose 750 nm spectra upon cleavage by HindIII, whereas a combinatorial library built with a 50/50 ratio of, respectively, AF-750 labeled, HindIII-based FND identimer and ATTO-550 HindIII-based FND identimer, will lose both 550 nm and 750 nm spectra upon cleavage, effectively doubling the mutual information that may be collected during one cleavage cycle.

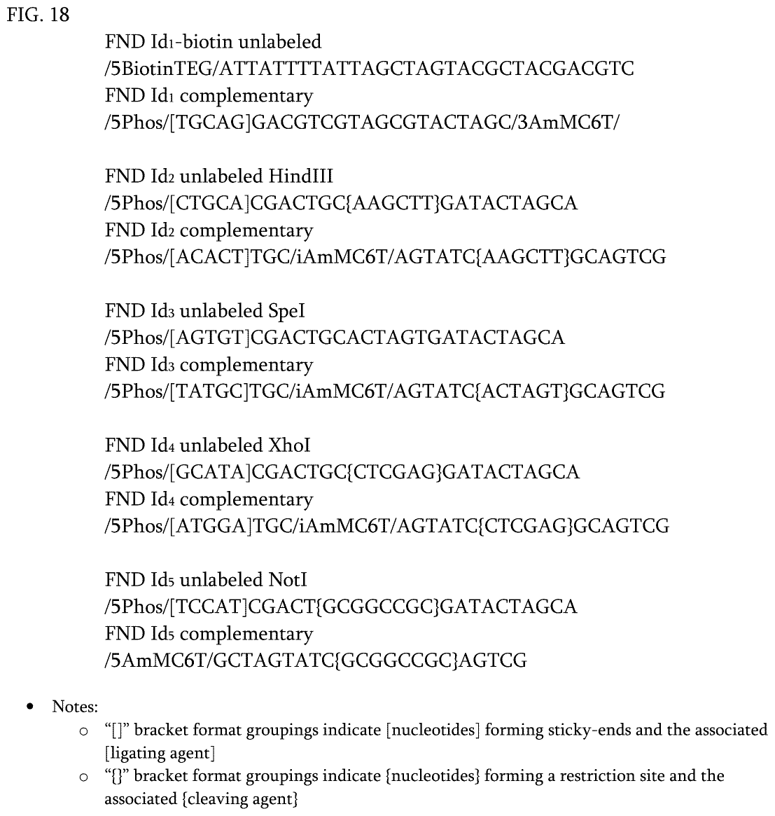

FIG. 18 is a textual representation of a set of five FND identimers (FND Id₁₋₅) having scaffold portions configured with orthogonal sticky-ends, orthogonal recognition moieties and cleavage sites as well as modified nucleotides. As shown in FIG. 18, each scaffold portion of FND Id₁₋₅ comprised an unlabeled single-stranded oligonucleotide configured to anneal with a complementary single-stranded, labeled oligonucleotide to form a duplex oligonucleotide with an unpaired, single-stranded overhang portion, or “sticky-end.” In some embodiments, the unpaired sticky-end may comprise 1 to 5 nucleotides. In some embodiments, the unpaired sticky-end may comprise more than 5 nucleotides. It was observed that more orthogonal sticky-end configurations became possible as the number of its nucleotides increased. In some embodiments, the ligation is a templated ligation. As used herein, “template ligation” or “templated ligation” collectively refer to ligation reactions that are specific to the correct complementary sticky ends. Skilled persons will understand that it would be possible to use as little as one base overhang, as that is the configuration used for NGS adapters commonly ligated onto DNA for sequencing in many existing kits known in the art. In some embodiments, an increased orthogonality is useful for allowing the simultaneous ligation of multiple identimer classes. Thus, in some embodiments, all identimer classes making up a set of identimer classes may be ligated together in a single reaction because each sticky-end pair in the set was configured to be orthogonal to each other. For example, an Id₁ identimer having a sticky-end configured to anneal to a sticky-end of an Id₂ identimer and remain orthogonal to a sticky-end of an Id₃ identimer allows for the selective ligation of Id₁ identimer to Id₂ identimer only. Skilled persons will understand that the process of sticky-end ligation facilitates the selective ligation of a pair of duplex oligonucleotides that have complementary sticky-ends.

### A. FND Identimer Scaffold Composition

FND Id₁₋₅ was produced to perform two orthogonal nuclease cleavage experiments producing combinatorial libraries from both single-label and dual-label FND identimer classes. The scaffold portion of FND Id₁ comprised an unlabeled SEQ ID NO: 17 configured to anneal to a labeled SEQ ID NO: 18. The nucleotides corresponding to positions 1 to 5 of SEQ ID NO: 18 comprised a sticky-end configured to anneal the sticky-end of SEQ ID NO: 19. Positions 1 and 25 were modified, respectively, by 5′ phosphorylation and to be a 3′ Amino Modifier C6 dT.

In some embodiments, a scaffold portion is configured to have a recognition moiety without a peptide or nucleotide sequence that reacts to the known cleaving agents to render it non-cleavable. In some embodiments, a non-cleavable identimer may be useful as a last-visualized identimer since some cleaving agents become less effective as steric hinderance increases. For example, it was observed that HindIII has reduced efficacy when cleaving dsDNA directly from a solid phase, such as a streptavidin bead. It was observed that, in certain instances, once a single label was observed following a cleavage step there was no need to perform the final cleavage step. This is analogous to having an uncleavable final Identimer token that is labeled. For example, in the case of FND Id₁₋₅ described herein, once a single label was observed following SpeI cleavage, there was no need to cleave the HindIII identimer tokens forming the remnant of the molecular barcode. Thus, in some embodiments, a last-visualized identimer may be configured to be cleavable, since once it has been identified by imaging, there would be no need to cleave it.

The scaffold portion of FND Id₂ comprised an unlabeled SEQ ID NO: 19 configured to anneal to a labeled SEQ ID NO: 20. The nucleotides corresponding to positions 1 to 5 of SEQ ID NO: 19 comprised a sticky-end configured to anneal the sticky-end of SEQ ID NO: 18. Positions 13 to 18 of SEQ ID NO: 19 comprised a HindIII recognition moiety and cleavage site. Position 1 of SEQ ID NO: 19 was modified by 5′ phosphorylation. The nucleotides corresponding to positions 1 to 5 of SEQ ID NO: 20 comprised a sticky-end configured to anneal to the sticky-end of SEQ ID NO: 21. Positions 16-21 of SEQ ID NO: 20 comprised a HindIII recognition moiety and cleavage site. Positions 1 and 9 of SEQ ID NO: 20 were modified, respectively, by 5′ phosphorylation and to be a Int Amino Modifier C6 dT.

The scaffold portion of FND Id₃ comprised an unlabeled SEQ ID NO: 21 configured to anneal to a labeled SEQ ID NO: 22. The nucleotides corresponding to positions 1 to 5 of SEQ ID NO: 21 comprised a sticky-end configured to anneal the sticky-end of SEQ ID NO: 20. Positions 13 to 18 of SEQ ID NO: 21 comprised a SpeI recognition moiety and cleavage site. Position 1 of SEQ ID NO: 21 was modified by 5′ phosphorylation. The nucleotides corresponding to positions 1 to 5 of SEQ ID NO: 22 comprised a sticky-end configured to anneal to the sticky-end of SEQ ID NO: 23. Positions 16-21 of SEQ ID NO: 22 comprised a SpeI recognition moiety and cleavage site. Positions 1 and 9 of SEQ ID NO: 22 were modified, respectively, by 5′ phosphorylation and to be Int Amino Modifier C6 dT.

The scaffold portion of FND Id₄ comprised an unlabeled SEQ ID NO: 23 configured to anneal to a labeled SEQ ID NO: 24. The nucleotides corresponding to positions 1 to 5 of SEQ ID NO: 23 comprised a sticky-end configured to anneal the sticky-end of SEQ ID NO: 22. Positions 13 to 18 of SEQ ID NO: 21 comprised a XhoI recognition moiety and cleavage site. Position 1 of SEQ ID NO: 21 was modified by 5′ phosphorylation. The nucleotides corresponding to positions 1 to 5 of SEQ ID NO: 24 comprised a sticky-end configured to anneal to the sticky-end of SEQ ID NO: 25. Positions 16 to 21 of SEQ ID NO: 22 comprised a XhoI recognition moiety and cleavage site. Positions 1 and 9 of SEQ ID NO: 24 were modified, respectively, by 5′ phosphorylation and to be a Int Amino Modifier C6 dT.

The scaffold portion of FND Id₅ comprised an unlabeled SEQ ID NO: 25 configured to anneal to a labeled SEQ ID NO: 26. The nucleotides corresponding to positions 1 to 5 of SEQ ID NO: 25 comprised a sticky-end configured to anneal the sticky-end of SEQ ID NO: 24. Positions 11 to 18 of SEQ ID NO: 25 comprised a NotI recognition moiety and cleavage site. Position 1 of SEQ ID NO: 25 was modified by 5′ phosphorylation. The nucleotides corresponding to positions 11 to 18 of SEQ ID NO: 26 comprised a NotI recognition moiety and cleavage site. Position 1 of SEQ ID NO: 26 was modified to be a 5AmMC6T.

As used herein, “3AmMC6T” refers to 3′ Amino Modifier C6 dT, a modified nucleotide available commercially from Integrated DNA Technologies, Inc. (IDT) (1710 Commercial Park, Coralville, Iowa 52241, USA). As used herein, “5′ Biotin-TEG” or “5BiotinTeg” collectively refer to a biotin molecule attached to a 15-atom, mixed polarity triethelyene glycol spacer, available commercially as a modification that can be installed during synthesis by IDT. Skilled persons will understand that 5′ Biotin-TEG may be incorporated at either the 5′ or 3′ end of an oligonucleotide. As used herein, “5Phos” refers to 5′ phosphorylation, such as, for example, the phosphorylation of an oligonucleotide at its 5′ end. Skilled persons will understand that 5′ Phosphorylation is needed if an oligonucleotide is used as a substrate for a DNA ligase enzyme. As used herein, “5AmMC6T” refers to 5′ Amino Modifier C6 dT, a modified nucleotide available commercially from Integrated DNA Technologies, Inc. (1710 Commercial Park, Coralville, Iowa 52241, USA).

### B. Single-Label and Dual-Label FND Identimer Detectable Label Composition

For the single-label orthogonal nuclease cleavage experiment, the detectable labels of FND Id₁₋₅ were configured accordingly: FND Id₁ was unlabeled; FND Id₂ comprised a single AF-750; FND Id₃ comprised a single AF-647; FND Id₄ comprised a single ATTO-550; and FND Id₅ comprised a single ATTO-488. As disclosed herein, each scaffold portion of FND Id₁₋₅ was labeled by NHS ester-mediated derivatization. AF-750 was used to label the 3′ Int Amino Modifier C6 dT of SEQ ID NO: 20 at position 9. AF-647 was used to label the Int Amino Modifier C6 dT of SEQ ID NO: 22 at position 9. ATTO-555 was used to label the Int Amino Modifier C6 dT of SEQ ID NO: 24 at position 9. ATTO-488 was used to label the 5′ Amino Modifier C6 dT of SEQ ID NO: 26 at position 1.

For the dual-label orthogonal nuclease cleavage experiment, the detectable labels of FND Id₁₋₅ were configured accordingly: FND Id₁ was unlabeled; FND Id₂ comprised approximately 50% AF-750 and approximately 50% ATTO-550; FND Id₃ comprised approximately 50% AF-647 and approximately ATTO-488; FND Id₄ comprised approximately 50% AF-647 and approximately 50% ATTO-550; and FND Id₅ comprised approximately 50% ATTO-488 and approximately 50% AF-750. As disclosed herein, each scaffold portion of FND Id₁₋₅ was labeled by NHS ester-mediated derivatization. AF-750 and ATTO-550 were used to label the 3′ Int Amino Modifier C6 dT of SEQ ID NO: 20 at position 9. AF-647 and ATTO-488 were used to label the Int Amino Modifier C6 dT of SEQ ID NO: 22 at position 9. AF-647 and ATTO-555 was used to label the Int Amino Modifier C6 dT of SEQ ID NO: 24 at position 9. ATTO-488 and AF-750 were used to label the 5′ Amino Modifier C6 dT of SEQ ID NO: 26 at position 1.

As used herein, “ATTO-488” is an ATTO fluorescent dye having an maximum absorption of 501 nm and a maximum fluorescence of 523 nm. Skilled persons will understand that ATTO-488 is excited more efficiently in a range of 480 nm to 515 nm. As used herein, “ATTO-550” is an ATTO fluorescent dye having a maximum absorption of 554 nm and a maximum fluorescence of 576 nm. Skilled persons will understand that ATTO-550 is excited more efficiently in a range of 540 nm to 565 nm. ATTO-488 and ATTO-550 are commercially available from ATTO-Tec GmbH (Martinshardt 7, 57074 Siegen; info@att-tec.com; Product No.: AD 488 and Product No.: AD550.)

As used herein, “AF-405” is an Alexa Fluor 405 dye, a blue-emitting synthetic fluorophore having an excitation peak at 401 nm and an emission peak at 421 nm. As used herein, “AF-647” is an Alexa Fluor 647 dye, a far-red fluorescent dye having an excitation suited for 594 nm or 633 nm laser lines. As used herein, “AF-750” is an Alexa Fluor 750 dye, a bright, near-infrared fluorescent dye having an excitation suited for 633 nm laser line or dye-pumped excitation. AF-405, AF-647, and AF-750 are commercially available from ThermoFisher Scientific, Inc. (168 Third Avenue, Waltham, MA 02451, USA; Cat. No. A30000 for AF-405; Cat. No. A20006 for AF-647; Cat. No. A20011 for AF-750).

In some embodiments, AF-405, AF-647, AF-750, ATTO-488, and ATTO-550 may be used interchangeably to produce different classes of Identimers. Skilled persons will understand that any NHS-conjugated fluorophore (NHS-fluorophore) may be used to label any free amino group by NHS ester-mediated derivatization. NHS-conjugated fluorophores are available commercially from the vendors provided herein.

### C. Single-Label and Dual-Label FND Identimer Detectable Label Construction

First, free amino (NH₂) groups on the labeled oligonucleotides of FND Id₁₋₅ (SEQ ID NO: 18, SEQ ID NO: 20, SEQ ID NO: 22, SEQ ID NO: 24, SEQ ID NO: 26) were labeled with fluorophores using 20 μl of 200 μM oligonucleotide and resuspended in 100 mM NaPO₄ (sodium phosphate buffer) at pH 8.5 and then mixed with 2 μl of 100 mM NHS-Fluorophore (e.g., NHS-ATTO-488, NHS-ATTO-550, NHS-AF-647, or NHS-AF-750 resuspended in anhydrous dimethylformamide (DMF)), in an overnight reaction at room temperature.

The labeled oligonucleotides were then diluted with 80 μl of 2× hybridization buffer (20 mM Tris pH 7.5, 1M NaCl, 1 mM EDTA) to a 100 μl volume to quench any remaining unreacted NHS groups. 100 μl of 40 μM labeled oligonucleotide was added to and mixed with its corresponding unlabeled oligonucleotide as described herein (i.e., SEQ ID NO: 17 mixed with SEQ ID NO: 18; SEQ ID NO: 19 mixed with SEQ ID NO: 20; SEQ ID NO: 21 mixed with SEQ ID NO: 22; SEQ ID NO: 23 mixed with SEQ ID NO: 24; SEQ ID NO: 25 mixed with SEQ ID NO: 26), for subsequent annealing.

### C. FND Identimer Scaffold Portion Annealing

Annealing was then performed by exposing all FND Id₁₋₅ scaffold portion oligonucleotide duplexes to 90° C. for approximately 5 minutes in a heat block and then placing the heat block on the lab bench until it reached room temperature (approximately 1.5 hours).

10 μl of biotinylated FND Id₁ scaffold portion oligonucleotide duplex was diluted 10-fold in 90 μl of hybridization buffer to 200 nM, and 100 μl of each differentially-labeled duplex was mixed with 100 μl of 0.2 mg/ml Dynabeads MyOne Streptavidin T1 beads (streptavidin beads) (available commercially from ThermoFisher Scientific, Inc. 168 Third Avenue, Waltham, MA 02451, USA; Cat. No. 65601) and then pre-washed 3× and resuspended in Hybridization buffer (prior to binding) rapidly by pipetting up and down to provide even coating at 1:1 (volume:volume). Binding was performed at 37° C. on a heat block over 30 minutes with occasional tapping of tubes. Conjugated streptavidin beads were then pulled on a magnet to remove unbound scaffold portion oligonucleotide duplexes. The solution was removed, and the conjugated streptavidin beads were washed: 3× in 300 μl of 2× hybridization buffer supplemented with 0.01% Tween-20 (commercially available from Sigma-Aldrich, Inc., PO Box 14508, St. Louis, MO 68178, USA; Cas No. 9005-64-5); 2× in 100 μl 1×T4 DNA ligase buffer (available commercially from New England Biolabs, Inc. (NEB), 240 County Road, Ipswich, MA 01938-2723, USA; info@neb.com); and, were then resuspended in 1× T4 DNA ligase buffer. The conjugated streptavidin beads were then sonicated for 3 minutes in a water bath sonicator to break up clusters of beads, and stored at 4° C. or on ice for use in downstream ligation reactions.

Ligation steps were performed at temperatures between room temperature or up to 37° C., in the presence of both T4 polynucleotide kinase (PNK) (commercially available from NEB; (5 μl/250 μl ligation reaction); Cat. No. M0201S or Mo201L) and T4 DNA ligase (commercially available from NEB; (5 μl/250 μl ligation reaction); Cat. No. M0202S, M0202T, M0202L, or M0202M) in 1×T4 DNA ligase buffer provided by NEB. On-bead ligations were performed using 0.1 mg/ml streptavidin beads coated with FND Id₁ scaffold portion oligonucleotide duplexes as described herein (washed 2× in 100 μl of 1× ligation buffer provided by NEB), for attachment of FND Id₂ scaffold portion oligonucleotide duplexes. All subsequent ligations of FND Id₃₋₅ scaffold portion oligonucleotide duplexes were performed under the same conditions. Washes between ligations were: 1 wash with 200 μl 2× hybridization buffer supplemented with 0.01% Tween-20, then 2 washes with 200 μl 1× hybridization buffer, then 2 washes with 100 μl of 1× ligation buffer provided by NEB.

In some embodiments, FND identimers may be ligated in solution prior to ligating to the streptavidin beads. For example, the set of FND Id₂ was pre-ligated with the set of FND Id₃ prior to ligation to the streptavidin beads via FND Id₁. This was performed by pre-mixing 5 μl of FND Id₂ scaffold portion oligonucleotide duplexes with 5 μl of FND Id₃ scaffold portion oligonucleotide duplexes (20 μM stock concentration), or pre-mixing 5 μl of FND Id₄ scaffold portion oligonucleotide duplexes with 5 μl of FND Id₅ scaffold portion oligonucleotide duplexes for example, with 25 μl of 10× ligase buffer (NEB), 205 μl of H₂O, and 5 μl of PNK (NEB) and 5 μl of T4 DNA ligase (NEB). These pre-mixed identimer ligations were performed at temperatures between room temperature and 37° C. 5 μl of streptavidin beads prepared as above (0.2 mg beads coated with Id₁ oligo duplexes, which were washed 3× with 200 μl 2× hybridization buffer supplemented with 0.01% Tween-20, then washed twice with 200 μl 1× hybridization buffer, then washed two more times with 100 μl of 1× ligation buffer provided by NEB, and resuspended in 50 μl of ligation buffer) were then pipetted into the bottom of new tubes for addition of the various 250 μl ligation reactions.

Ligations were performed in the presence of PNK and T4 DNA ligase. Between each round of ligation, extensive washing was performed using 0.5-1 mM EDTA to quench any remaining ligase activity, high salt (between 500 mM-1M NaCl) to remove ligase from dsDNA duplexes, and detergent (0.01% Tween-20) to aid in ligase enzyme removal from duplexes and to prevent clumping of beads; the latter could lead to uneven coating of beads during subsequent ligations, so to further prevent this, beads were sonicated for at least 3 minutes prior to imaging, and prior to being exposed to any enzyme catalyzed reactions. For further details, see methods section.

After all ligations for encoding were complete, the streptavidin beads were washed 3× with 200 μl 2× hybridization buffer supplemented with 0.01% Tween-20, 2× with 200 μl 1× hybridization buffer, resuspended in 20 μl of 1× hybridization buffer, sonicated for 3 minutes, and loaded into a flow cell containing a biotin-modified surface for immobilization and subsequent cleavage experiments.

### D. Results

### Encoding Single-Label and Dual-Label FND Identimer-Based Molecular Barcodes

As shown in Example 5, FND identimer-based molecular barcodes were constructed by ligation of differentially-labeled, pre-hybridized oligonucleotide duplexes. Molecular barcodes having 4 different distinguishable labels were constructed from 5 segments (i.e., 5 identimer tokens per molecular barcode) using a combinatorial ligation strategy. As disclosed herein, a set of FND identimers was bound to streptavidin beads via a 5′-biotin modification on one of the oligonucleotide strands of the scaffold portion oligonucleotide duplexes in the set. (Skilled persons will understand that a 5′-biotin modification may be applied to either strand of an oligonucleotide duplex.) Here, the scaffold portion oligonucleotide duplexes of FND Id₁ bound to the streptavidin beads was unlabeled, and generated a 5′ overhang (phosphorylated) compatible for ligation to the scaffold portion oligonucleotide duplexes of FND Id₂.

As disclosed in Example 5, the scaffold portions of FND Id₂ were labeled with AF-750 and comprised both an enzyme-accessible HindIII cleavage site and a 5′ overhang (phosphorylated) compatible for ligation to the scaffold portion oligonucleotide duplexes of FND Id₃. Following ligation of the FND Id₂ to FND Id₁, a sample of these streptavidin beads were immobilized in a flow cell for imaging. Images of the streptavidin beads bound to molecular barcodes containing FND Id₁ and FND Id₂ were taken in 4 fluorescence channels, and intensity values were plotted as shown in FIG. 19. FIG. 19A represents a streptavidin bead labeled with a first labeled dsDNA identimer segment (Id₁/HindIII-AF750) attached through ligation to an unlabeled dsDNA that is attached to the bead through a biotin moiety. FIG. 19B represents a streptavidin bead labeled with a second labeled dsDNA identimer segment (Id₂/SpeI-AF647). FIG. 19C represents a streptavidin bead labeled with a third labeled dsDNA identimer segment (Id₃/XhoI-ATT0550). FIG. 19D represents a streptavidin bead labeled with a fourth labeled dsDNA identimer segment (Id₄/NotI-ATT0488). FIG. 20A represents un-cleaved beads imaged in four fluorescence channels; 647 nm (upward diagonal stripes), 550 nm (downward diagonal stripes), 488 nm (horizontal stripes) and 750 nm (dotted), and mean intensity values obtained for each fluorophore were plotted to the right.

Clear AF-750 label detection was observed on beads containing the FND Id₁/FND Id₂ ligation products (FIG. 19A) suggesting the ligation reaction was efficient, while little to no signal was observed in any of the other fluorescence channels. The streptavidin beads bound to molecular barcodes comprising ligated (i.e., encoded) FND Id₁ and FND Id₂ identimer tokens were then subjected to another round of ligation, whereby the FND Id₃ were ligated (i.e., concatenated) to the growing molecular barcode. As disclosed in Example 5, the scaffold portions of the FND Id₃ were labeled with AF-647 and comprised both an enzyme-accessible SpeI cleavage site and a 5′ overhang (phosphorylated) compatible for ligation to the scaffold portion oligonucleotide duplexes of FND Id₄. Following ligation of the FND Id₃, a sample of the beads were immobilized in a flow cell for imaging. Images of streptavidin beads bound to molecular barcodes comprising ligated (i.e., encoded) FND Id₁/FND Id₂/FND Id₃/identimer tokens were taken in 4 fluorescence channels, and intensity values were plotted (FIG. 19). AF-750 and AF-647 were detected on streptavidin beads bound to molecular barcodes containing the ligated FND Id₁/FND Id₂/FND Id₃/identimer tokens (FIG. 19B) suggesting that the ligation of the FND Id₃ was successful. No signal was observed in the other two fluorescent channels (488 nm or 550 nm emissions) when imaging streptavidin beads bound to the ligated FND Id₁/FND Id₂/FND Id₃/identimer tokens. The streptavidin beads bound to molecular barcodes comprising the ligated (i.e., encoded) FND Id₁/FND Id₂/FND Id₃/identimer tokens were then subjected to another round of ligation, whereby the FND Id₄ were ligated (i.e., concatenated) to the growing molecular barcode. As disclosed in Example 5, the scaffold portions of the FND Id₄ were labeled with ATTO-550 and comprised both an enzyme-accessible XhoI restriction site, and a 5′ overhang (phosphorylated) compatible for ligation to the scaffold portion oligonucleotide duplexes of FND Id₅. Following ligation of the FND Id₄, a sample of the streptavidin beads were immobilized in a flow cell for imaging. Images of the streptavidin beads bound to molecular barcodes containing the ligated (i.e., encoded) FND Id₁/FND Id₂/FND Id₃/FND Id₄/identimer tokens were taken in 4 fluorescence channels, and intensity values were plotted (FIG. 19). AF-750, AF-647, and ATTO-550 were clearly observed on the streptavidin beads bound to molecular barcodes comprising FND Id₁/FND Id₂/FND Id₃/FND Id₄/identimer tokens (FIG. 19C) suggesting ligation of the FND Id₄ was successful. No signal was observed in the remaining fluorescent channel (488 nm emission) when imaging the streptavidin beads bound to molecular barcodes comprising FND Id₁/FND Id₂/FND Id₃/FND Id₄/identimer tokens. The streptavidin beads bound to molecular barcodes comprising FND Id₁/FND Id₂/FND Id₃/FND Id₄/identimer tokens were then subjected to another round of ligation, whereby the FND Id₅ were ligated (i.e., concatenated) to the growing molecular barcode. As disclosed in Example 5, the scaffold portions of the FND Id₅ were labeled with ATTO-488 and comprised both an enzyme-accessible NotI restriction site (i.e., cleavage site). Following ligation of the FND Id₅, a sample of the streptavidin beads were immobilized in a flow cell for imaging. Images of streptavidin beads bound to molecular barcodes comprising FND Id₁/FND Id₂/FND Id₃/FND Id₄/FND Id₅/identimer tokens were taken in 4 fluorescence channels, and intensity values were plotted (FIG. 19). Signal for all four labels (AF-750, AF-647, ATTO-550 and ATTO-488) were observed on the streptavidin beads bound to molecular barcodes comprising FND Id₁/FND Id₂/FND Id₃/FND Id₄/FND Id₅/identimer tokens (FIG. 19D) suggesting ligation of the FND Id₅ was successful. These results suggest that differentially labeled chains of identimers (i.e., molecular barcodes) can be constructed using a splitting and pooling approach to generate combinatorial libraries of labeled beads. The ligated identimer chains shown in FIG. 1, as well as other chains containing other various label combinations were made in the same way (generated on beads), and beads were immobilized in lanes of a flow cell for subsequent decoding experiments.

### Decoding Single-Label and Dual-Label FND Identimer-Based Molecular Barcodes

In some embodiments, the three dimensional arrangement is linear, having one or two open ends. In some embodiments, the three dimensional arrangement is circular, having no open ends. In some embodiments, the three dimensional arrangement is a hairpin formation, having a looped end and an open end. Skilled persons will understand that sticky-end ligation of two or more dsDNA oligonucleotides will generally form a linear chain of segments, each segment ligated together and at least one end, to form a chain.

FIG. 20B represents mean intensity of beads following exposure to the NotI enzyme. FIG. 20C represents mean intensity of beads following exposure to the XhoI enzyme. FIG. 20D represents mean intensity of beads following exposure to the SpeI enzyme. FIGS. 20A-20D show the decoding of a molecular barcode formed and encoded in a three dimensional arrangement by the concatenation of a set of one or more FND Identimers. As shown in FIGS. 20A-20D, molecular barcodes, were formed and encoded from linear, segmented chains of ligated FND Id₁₋₅ identimer tokens and streptavidin beads. As shown in FIGS. 20A-20D, the FND Id₁ identimer tokens were unlabeled; the FND Id₂ identimer tokens were labeled with ATTO-550 and encoded an enzyme-accessible HindIII recognition moiety; the FND Id₃ identimer tokens were labeled with AF-647 and encoded an enzyme-accessible SpeI recognition moiety; the FND Id₄ identimer tokens were labeled with AF-750 and encoded an enzyme-accessible XhoI recognition moiety; and the FND Id₅ identimer tokens were labeled with ATTO-488 and encoded an enzyme-accessible NotI restriction site. For decoding, streptavidin beads bearing the described identimer chain (and label combination) were immobilized on the surface of a flow cell, and were imaged within a single lane of the flow cell. In this decoding experiment, all of the beads in any given field-of-view (FOV) were bearing the same identimer chain. Streptavidin beads in the flow cell lane were imaged prior to being exposed to any restriction enzymes, whereby signal for all four detectable labels was observed (FIG. 20A). To demonstrate controlled removal of one label per cycle, the streptavidin beads in the flow cell lane were first contacted with a solution containing 200 U of NotI enzyme (substantially removed of glycerol by desalting) in 1× CUTSMART® buffer provided by New England Biolabs (Cat. No. B7204), and incubated for 30 minutes at 37° C. in a temperature-controlled heat-block chamber (off of the microscope).

Following incubation with the NotI enzyme, the flow cell lane was flushed with at least 100 μl of 1× CUTSMART® buffer, and the flow cell was placed back on the microscope stage. Images of the same flow cell lane were acquired in all 4 fluorescence channels (from a different FOV, as tracking individual beads was not required for this experiment). Upon imaging, clear removal of the NotI-cleavable label (ATTO-488) was observed from this first cycle of decoding by orthogonal cleavage (FIG. 20B). Signal observed in all other channels (550 nm, 647 nm, and 750 nm) remained high, suggesting the cleavage by NotI was specific and orthogonal with respect to other encoded restriction sites within the identimer chain. The flow cell was then removed from the microscope, and beads were contacted with a solution containing 400 U of XhoI enzyme (substantially removed of glycerol by desalting) in 1× CUTSMART® buffer provided by New England Biolabs (NEB), and incubated for 30 minutes at 37° C. in a temperature-controlled heat-block chamber (off of the microscope). Following incubation with the XhoI enzyme, the flow cell lane was flushed with at least 100 μl of 1× CUTSMART® buffer, and the flow cell was placed back on the microscope stage. Images of the same flow cell lane were then acquired in all 4 fluorescence channels. Upon imaging, clear removal of the XhoI-cleavable label (AF-750) was observed from this second cycle of decoding by orthogonal cleavage (FIG. 20C). In these images, streptavidin beads have been removed of both FND Id₅- and FND Id₄-associated labels, suggesting that like NotI, the XhoI enzyme was also able to perform specific and orthogonal cleavage only in response to its encoded restriction site. The flow cell was then removed from the microscope, and beads were contacted with a solution containing 300 U of SpeI enzyme (substantially removed of glycerol by desalting) in 1× CUTSMART® buffer provided by New England Biolabs (NEB), and incubated for 30 minutes at 37° C. in a temperature-controlled heat-block chamber (off of the microscope). Following incubation with the SpeI enzyme, the flow cell lane was flushed with at least 100 μl of 1× CUTSMART® buffer, and the flow cell was placed back on the microscope stage. Images of the same flow cell lane were then acquired in all 4 fluorescence channels. Upon imaging, it was observed that streptavidin beads subjected to this third cycle of decoding by orthogonal cleavage were removed of SpeI-cleaved AF-647 operatively connected to FND Id₃ (FIG. 20d) and strong signal for only a single remaining label (ATTO-550) was observed. The final label remaining on the bead was clearly distinguishable in images, and therefore subsequent cleavage of the HindIII-cleavable label (FND Id₂) was not necessary. Thus, differentially labeled combinations of identimer chains may be encoded by a split-and-pool ligation strategy, and may subsequently be decoded in massive parallel fashion via cycles of orthogonal cleavage reactions.

### Decoding FND Identimer Tokens Having Single and Dual (Mixed) Detectable Labels

As shown in FIG. 21B, molecular barcodes comprise one unlabeled FND identimer token (FND Id₀) and four dual-labeled FND Identimer tokens (FND Id₁-550/750, FND Id₂-488/647, FND Id₃-750/488, and FND Id₄-488)

FIG. 21A presents a bar graph representing OCS results obtained for single-label dsDNA identimer chains where the order of the cycles was reversed for analysis.

FIG. 21B presents a bar graph representing OCS results obtained for mixed-label dsDNA identimer chains where the order of the cycles was reversed for analysis.

FIGS. 21A and 21B are, respectively, bar graphs showing the decoding of molecular barcodes comprised of differentially labeled FND Identimer tokens having single and dual (mixed) detectable labels. As shown in FIG. 21A, molecular barcodes comprising four single-label FND identimer tokens (encoded using FND identimer classes: FND Id₁-550, FND Id₂-750, FND Id₃-647, and FND Id₄-488) were constructed and then decoded to demonstrate the combinatorial library theoretical capacity of single-label FND Identimer-based molecular barcodes. For example, to construct a set of molecular barcodes comprising dual-labeled identimer tokens, a FND Id₂-488 was mixed at an equimolar ratio with a FND Id₂-550 to generate a total of 10 visually-discernable combinations of detectable labels per dual-label identimer (as opposed to 4 different possibilities per single-label identimer). The visually-discernable combinations for each dual-label FND Identimer that were mixed for constructing dual-labeled identimer chains were 488/488, 488/550, 488/647, 488/750, 550/550, 550-/647, 550/750, 647/647, 647-/750, and 750/750. For these experiments, pre-determined chain configurations (i.e., pre-determined single labels or mixtures of labels at each identimer position in the chain) were constructed on streptavidin beads as described previously (i.e., by rounds of ligation with washes in between). However, single-label chains constructed by in-solution ligation of all identimer segments comprising a chain within a single ligation reaction, then subsequently captured onto streptavidin beads, showed no difference in performance (data not shown). Average bead intensity across greater than 100 beads in each field of view were acquired for streptavidin beads bearing single-label and dual-label identimer chains in all four fluorescence channels at each cycle. Images were acquired for uncut streptavidin beads, streptavidin beads subsequently cleaved with NotI (cycle 1), streptavidin beads subsequently cleaved with XhoI (cycle 2), and streptavidin beads subsequently cleaved with SpeI (cycle 3). Images were then analyzed in reverse-chronological order. For example, images acquired following cycle 3 (cleavage by SpeI) were analyzed as the first images in the series; images acquired following cycle 2 (cleavage by XhoI) were analyzed as the second images in the series; images acquired following cycle 1 (cleavage by NotI) were analyzed as the third images in the series; and uncleaved beads were analyzed as the fourth images in the series. While in reverse chronological order, intensity values for all four fluorescence channels obtained in the “previous” image were subtracted from the “next” image to enable cycle by cycle determination of labels associated with each identimer segment. For example, intensity values obtained for all four fluorescent channels from images acquired following SpeI cleavage were subtracted from intensity values obtained for all four fluorescent channels from images acquired following XhoI cleavage, and this analysis was performed for each cycle in the series. Following analysis, intensity values obtained in all four fluorescence channels were plotted for both single- and dual-labeled chains at each cycle. FIG. 21A shows the sequence or order of labels released from beads bearing single label identimer chains. FIG. 21B shows the order released from streptavidin beads bearing dual-labeled chains. The order (i.e., sequence) of labels released across all cleavage cycles in the single-label experiment was readily resolved; only labeled FND identimer tokens are numbered in the graph (Id₀ is unlabeled and attached to the bead). An imaging error prevented identification of Id₃ in the dual-labeled experiment, but labels attached to flanking identimer segments in the chain(s) were easily resolved. The dsDNA identimer chain sequence that was decoded in the single label experiment was Id₁-550, Id₂-750, Id₃-647, and Id₄-488. The dsDNA identimer chain sequence that was decoded in the dual label experiment was Id₁-550/750, Id₂-488/647, Id₃ was not determined, and Id₄-750/488. These results suggest that combinatorial libraries of fluorescently labeled beads can be encoded and decoded using this approach. For example, the theoretical diversity of such combinatorial libraries (developed based on the experiment shown), would be 256 for single-label chains, and 10,000 for dual-label chains. To decode such libraries at their maximum theoretical diversity would require tracking of individual streptavidin beads.

### Decoding FND Identimer-Based Molecular Barcodes: Tracking 9 Individual Beads Over 4 Images (Single Label Per Identimer, 3 Cleavage Cycles)

As disclosed previously herein, molecular barcodes comprising FND Id₁-550, FND Id₂-750, FND Id₃-647, and FND Id₄-488 tokens (constructed by in-solution ligation of all identimers within a single ligation reaction and then captured onto beads) showed no obvious difference in performance (qualitative and quantitative assessments from various decoding experiments) when compared to FND Identimer-based molecular barcodes constructed step-wise by ligation of each individual identimer with washes in between. Therefore, whole single-label FND Identimer-based molecular barcodes built for the single bead tracking experiment (as shown in FIG. 22) were pre-ligated in solution before being used for coating beads.

To demonstrate the tracking of single beads across all cleavage cycles, streptavidin beads conjugated to molecular barcodes comprising a single, pre-determined sequence of labeled FND Identimers tokens were immobilized in a flow cell and imaged before and after exposure to each cleavage agent (NotI in cycle 1, XhoI in cycle 2, and SpeI in cycle 3). Following imaging of all cycles, 9 individual beads were selected for tracking, images were analyzed in reverse chronological order, values from the “previous” cycle were subtracted as described above, and the resulting intensity values were plotted for all 9 beads at each cycle.

FIG. 22A represents the first of six I beads bearing the same single-label identimer chain sequence used to generate data from individual beads in a FOV tracked over 3 cycles of an OCS experiment. FIG. 22B represents the second of six I beads bearing the same single-label identimer chain sequence used to generate data from individual beads in a FOV tracked over 3 cycles of an OCS experiment. FIG. 22C represents the third bead bearing the same single-label identimer chain sequence used to generate data from individual beads in a FOV tracked over 3 cycles of an OCS experiment. FIG. 22D represents the fourth bead bearing the same single-label identimer chain sequence used to generate data from individual beads in a FOV tracked over 3 cycles of an OCS experiment. FIG. 22E represents the fifth bead bearing the same single-label identimer chain sequence used to generate data from individual beads in a FOV tracked over 3 cycles of an OCS experiment. FIG. 22F represents the sixth bead bearing the same single-label identimer chain sequence used to generate data from individual beads in a FOV tracked over 3 cycles of an OCS experiment.

FIG. 22 shows FND Identimer “orthogonal cleavage sequencing” (OCS) data obtained from the 9 individually tracked streptavidin beads. All streptavidin beads tracked in this experiment showed the expected (correct) order of fluorophore release upon exposure to the pre-determined order of orthogonal cleavage agents. These results suggest that streptavidin beads conjugated to FND Identimer-based molecular barcodes comprising many different uniquely-labeled FND identimer token sequences can be resolved in highly parallel fashion.

### Encoding and Decoding of a 256-Member Combinatorial Bead Library: FND Identimer Molecular Barcodes Comprising Different Color Codes

To demonstrate the encoding of many streptavidin beads in parallel, single-label FND Identimers were incorporated into chains containing differential label combinations in random fashion. Combinatorial molecular barcode construction was performed by splitting and pooling of streptavidin beads in between rounds of FND Identimer ligation steps. To generate a library of fluorescent streptavidin beads with a total diversity of 256, 4 different labels were placed on each of four FND Identimer classes (FND(256) Id₁₋₄) to produce 256 possible label combinations. FND(256) Id₀ was unlabeled and first attached to the streptavidin beads to act as an acceptor for ligation of FND(256) Id₁ (described previously herein when constructing single-label chains). FND(256) Id₁₋₃ were configured to have, respectively, HindII, SpeI, XhoI recognition moieties. More specifically, FND(256) Id₁₋₄ were first labeled with all 4 fluorophores (ATTO-488, ATTO-550, AF-647, and AF-750) by the labeling methods disclosed in Example 5. Each labeled oligonucleotide duplex was kept in separate wells of a standard 96-well plate. In this way, during each round of splitting and pooling, 4 differentially labeled options were available for each FND identimer. In the first round of molecular barcode construction, 0.2 mg/ml beads coated with FND(256) Id₀ were split into 4 wells and exposed to a solution containing 1×T4 DNA ligase buffer supplemented with T4 polynucleotide kinase (PNK), T4 DNA ligase, and one of the four differentially labeled FND(256) Id₁ (i.e., FND(256) Id₁-488, FND(256) Id₁-550, FND(256) Id₁-647, or FND(256) Id₁-750) at a 300 nM concentration. After an incubation (approximately 15 to 20 minutes) to promote efficient ligation of FND(256) Id₁, the streptavidin beads (magnetic) were washed several times in the same wells (in which they were ligated) with a solution used for quenching the ligation reaction, and for washing away un-ligated FND(256) Id₁. Streptavidin beads conjugated to the four differentially-labeled FND(256) Id₁ were then pooled into the same tube for even mixing. Streptavidin beads were then split into 4 wells and exposed to ligation solution containing each of the 4 differentially-labeled options available for FND(256) Id₂. This splitting and pooling procedure was repeated for ligation of all four identimer classes of FND(256) Id₁₋₄ to generate a library of 256 different beads. Using the OCS procedure described previously herein, single beads from the library were tracked, images were analyzed in reverse chronological order as described above (with “previous” cycle subtraction), and the fluorescence values obtained from imaging in all four channels were plotted for each cycle.

FIG. 23 shows OCS sequencing reads for 3 individual streptavidin beads from the 256-member library. The order of fluorophore release from the 3 beads shown in FIG. 23 was readily resolved in this experiment. Taken together with the dual-labeling experiment, these data suggest that a library of 10,000 beads could be encoded and decoded using the methods described here. With the addition of more identimers in the chain, or with the use of additional individually detectable labels (such as quantum dots for example), much larger libraries can be encoded and decoded using OCS.

### Example 6 Encoding and Decoding Ring Identimer-Based Molecular Barcodes

### A. Ring Identimer Composition and Construction

As disclosed in Example 6, three identimer classes (Ring Id₁₋₃) were constructed having scaffold portions comprising ligated rings of dsDNA (referred to herein as “Ring Identimers” or “Ring Id”) and were used to construct a set of Ring Id-based molecular barcodes. The scaffold portions of each of Ring Id₁₋₃ were configured to comprise one or more restriction sites and amino-modified bases for attachment to a streptavidin bead (via NHS-LC-biotin; commercially available from Sigma-Aldrich, Inc., PO Box 14508, St. Louis, MO 68178, USA; CAS No.: 35013-72-0), and NHS-fluorophores (available commercially from ThermoFisher Scientific, Inc. 168 Third Avenue, Waltham, MA 02451, USA; Cat. No. 65601). All oligonucleotide-streptavidin conjugations were carried out as described previously herein.

The scaffold portion of Ring Id₁ comprised a Hp_Dual_NH2_XhoI oligonucleotide (SEQ ID NO: 27) and a Hp_iNH2_XhoI bead oligonucleotide (SEQ ID NO: 28). The nucleotide corresponding to position 1 of SEQ ID NO: 27 was modified by 5′ phosphorylation. Positions 14 and 20 of SEQ ID NO: 27 were Int Amino Modifier C6 dT modified nucleotides. Positions 34 to 37 of SEQ ID NO: 27 comprised a sticky-end configured to anneal to the sticky end of SEQ ID NO: 28. Positions 13 to 22 of SEQ ID NO: 27 comprised a stem-loop feature. The nucleotide corresponding to position 1 of SEQ ID NO: 28 was modified by 5′ phosphorylation. Position 17 of SEQ ID NO: 28 was an Int Amino Modifier C6 dT modified nucleotide. Positions 34 to 37 of SEQ ID NO: 28 comprised a sticky-end configured to anneal to the sticky end of SEQ ID NO: 27. Positions 13 to 21 of SEQ ID NO: 27 comprised a stem loop feature.

The scaffold portion of Ring Id₂ comprised a Hp_Dual_NH2_SpeI oligonucleotide (SEQ ID NO: 29) and a Hp_iNH2_SpeI bead oligonucleotide (SEQ ID NO: 30). The nucleotide corresponding to position 1 of SEQ ID NO: 29 was modified by 5′ phosphorylation. Positions 14 and 20 of SEQ ID NO: 29 were Int Amino Modifier C6 dT modified nucleotides. Positions 34 to 37 of SEQ ID NO: 29 comprised a sticky-end configured to anneal to the sticky end of SEQ ID NO: 30. Positions 13 to 21 of SEQ ID NO: 29 comprised a stem loop feature. The nucleotide corresponding to position 1 of SEQ ID NO: 30 was modified by 5′ phosphorylation. Position 17 of SEQ ID NO: 30 was an Int Amino Modifier C6 dT modified nucleotide. Positions 34 to 37 of SEQ ID NO: 30 comprised a sticky-end configured to anneal to the sticky end of SEQ ID NO: 29. Positions 13 to 21 of SEQ ID NO: 30 comprised a stem loop feature.

The scaffold portion of Ring Id₃ comprised a Hp_Dual_NH2_NotI oligonucleotide (SEQ ID NO: 31) and a Hp_iNH2_NotI bead oligonucleotide (SEQ ID NO: 32). The nucleotide corresponding to position 1 of SEQ ID NO: 31 was modified by 5′ phosphorylation. Positions 16 and 22 of SEQ ID NO: 31 were Int Amino Modifier C6 dT modified nucleotides. Positions 38 to 41 of SEQ ID NO: 31 comprised a sticky-end configured to anneal to the sticky end of SEQ ID NO: 32. Positions 13 to 21 of SEQ ID NO: 31 comprised a stem loop feature. The nucleotide corresponding to position 1 of SEQ ID NO: 32 was modified by 5′ phosphorylation. Position 17 of SEQ ID NO: 32 was an Int Amino Modifier C6 dT modified nucleotide. Positions 34 to 37 of SEQ ID NO: 32 comprised a sticky-end configured to anneal to the sticky end of SEQ ID NO: 31. Positions 13 to 21 of SEQ ID NO: 32 comprised a stem loop feature.

A set of Hp_iNH2_XhoI bead oligonucleotides, a set of Hp_iNH2_SpeI bead oligonucleotides and, a set of Hp_iNH2_NotI bead oligonucleotide (collectively referred to as “Hp_iNH2 oligonucleotides”) were synthesized in preparation for conjugation to a set of streptavidin beads. The Hp_iNH2 oligonucleotides were combined with amine-reactive biotin (NHS-LC-biotin) to conjugate the biotin to the Hp_iNH2 oligonucleotides at their free amines (i.e., at nucleotides corresponding to position 17 for all three Hp_iNH2 oligonucleotides. Each set of the biotinylated Hp_iNH2 oligonucleotides was suspended to final concentration of 200 nM.

A set of Hp_Dual_NH2_XhoI oligonucleotides, a set of Hp_Dual_NH2_SpeI oligonucleotides, and a set of Hp_Dual_NH2_NotI oligonucleotides (referred to collectively as “Hp_Dual_NH2 oligonucleotides”) were synthesized and were labeled with fluorophores in preparation for ligation to their respective Hp_Dual_NH2 oligonucleotides. Amine-reactive ATTO-550 (NHS-ATTO-550) was combined with the set of Hp_Dual_NH2_XhoI oligonucleotides to dually label them at the nucleotides corresponding to positions 14 and 20. Amine-reactive ATTO-647 (NHS-ATTO-647) was combined with the set of Hp_Dual_NH2_SpeI oligonucleotides to dually label them at the nucleotides corresponding to positions 14 and 20. Amine-reactive ATTO-488 (NHS-ATTO-488) was combined with the set of Hp_Dual_NH2_NotI oligonucleotides to dually label them at the nucleotides corresponding to positions 16 and 22. Each set of the labeled Hp_Dual_NH2 oligonucleotides was suspended to final concentration of 200 nM.

To form and encode the set of Ring Id-based molecular barcodes, all three sets of biotinylated Hp_iNH2 oligonucleotides were first bound to the set of streptavidin beads by methods previously described herein. The set of streptavidin beads was washed to remove unbound oligonucleotides (3× with 20002× hybridization buffer supplemented with 0.01% Tween-20, then washed twice with 200 μl 1× hybridization buffer), and then washed two more times with 100 μl of 1× ligation buffer provided by NEB, resuspended in 50 μl of ligation buffer, and sonicated for 3 minutes to break up any clumps of beads prior to ligation reactions.

Ligation reactions were carried out as described previously herein, using 0.2 mg/ml of streptavidin beads coated with members from all three sets of Hp_iNH2 oligonucleotides and introducing each set of Hp_Dual_NH2 oligonucleotides in a separate round of ligation. In other words, the set of streptavidin beads was ligated: firstly to the set of Hp_Dual_NH2_XhoI oligonucleotides via stick-end ligation to bead-conjugated Hp_iNH2_XhoI bead oligonucleotides in a first ligation cycle; secondly to the set of Hp_Dual_NH2_SpeI oligonucleotides via sticky-end ligation to bead-conjugated Hp_iNH2_SpeI bead oligonucleotides in a second ligation cycle; and then thirdly to the set of Hp_Dual_NH2_NotI oligonucleotides via Hp_iNH2_NotI bead oligonucleotides in a third ligation cycle to complete the construction of Ring Id₁₋₃ and form the set of a set Ring Id-based molecular barcodes. Thus, the three-dimensional arrangement of each Ring Id-based molecular barcodes comprised sets of encoded Ring Id₁₋₃ tokens conjugated and a single streptavidin bead.

### B. Decoding Ring Id-Based Molecular Barcodes

After the three ligation rounds of encoding were complete, the streptavidin beads were washed 3× with 200 μl 2× hybridization buffer supplemented with 0.01% Tween-20, 2× with 200 μl 1× hybridization buffer, resuspended in 20 μl of 1× hybridization buffer, sonicated for 3 minutes, and loaded into a flow cell containing a biotin-modified surface for immobilization and subsequent cleavage experiments.

In some embodiments, OCS-compatible streptavidin bead libraries may be used for the co-encoding of other molecules whose components are coordinated with the OCS code. For example, barcodes within nucleic acid capture oligonucleotides used in NGS workflows (as described herein, or as a separate molecule attached to the same bead), and this can be accomplished by coordinate ligation. In some embodiments, identimer configurations that can withstand synthetic chemical reactions are useful to enable encoding of chemical libraries. OCS-compatible libraries can be constructed from scaffolds portions comprised of polymers other than DNA to address this (as described previously herein). Skilled persons will understand DNA is largely susceptible to degradation when its linear 5′- and 3′-ends exposed and is therefore more protected from degradation by exposure to some chemical reactions if circularized.

To further explore the efficacy of different identimer three-dimensional arrangements in OCS workflows, labeled combinations of circular ssDNA rings containing long regions of dsDNA encoding different restriction enzyme recognition sites were constructed step-wise on beads as described previously herein (FIG. 25). Three different ssDNA hairpin oligonucleotides Hp_iNH2 oligonucleotides were attached to a streptavidin bead, each containing 5′ overhangs that were compatible for ligation with only one of three different labeled hairpin oligonucleotides. One labeled hairpin was introduced at a time in ligation solution as described above, over 3 rounds of construction (encoding). Images were taken of beads immobilized in a flow cell in the relevant fluorescence channels (to acquire data for 488 nm, 550 nm, and 647 nm emissions) following each round of ring identimer construction. Intensity values were obtained from averages of many streptavidin beads in a given FOV, and plotted for each label beside raw images. It was clear from imaging that beads gained fluorescence intensity in only the expected channel upon step-wise ligation with each labeled hairpin.

This process can be repeated in a splitting and pooling procedure as described previously, with ligation steps separated by washing steps prior to each step of pooling. These results show that identimers can be constructed as DNA rings, and that these structures can be encoded in combinations.

To test the ring structures in an OCS workflow, beads containing three formed ring identimers were immobilized in a flow cell and exposed to a solution containing 300 U of the SpeI enzyme (FIG. 8A). Images were taken before and after cleavage by SpeI, and total intensity values obtained from beads were plotted for the three relevant fluorescence channels (FIG. 8B). Indeed, once ligated, the circular product comprises a fully formed, enzyme-accessible dsDNA restriction site compatible for cleavage by SpeI. Taken together, these results show that identimers can be constructed as rings of DNA, and that they can be encoded in combinations to make OCS-compatible libraries.

### Example 7 Encoding and Decoding Hairpin Identimer-Based Molecular Barcodes

To explore efficacy of different identimer three dimensional structures in OCS workflows, fluorescent ssDNA hairpin identimers comprising two differential labels separated by an enzyme-accessible SpeI cleavage site were constructed (FIG. 24A) by ligation of a labeled and biotinylated dsDNA acceptor with a dual-labeled hairpin by methods previously disclosed herein. Images were acquired from streptavidin beads (immobilized in a flow cell as described previously) that were coated FSH Identimers in both fluorescence channels, before and after (FIG. 24B) exposure to 300 U of SpeI in 1× CUTSMART® Buffer (NEB). Acquired images show clear removal of the AF-750 following 5 minutes of exposure to 300 U of the SpeI enzyme at 37° C., while strong signal from the AF-647 label remained on the bead. Values from many beads in each FOV were summed and averaged (individual beads were not tracked here), then fluorescence intensity values were corrected to percentage values for visual representation of total % signal loss (750 nm emission) from beads following SpeI cleavage (FIG. 24C). The loss in AF-750 signal following cleavage was ˜82% in this experiment. These results suggest that the hairpin structure designed here can function as an identimer of two segments, allowing for combinations of different hairpins containing different combinations of restriction sites and visually-discernable labels to be used in the construction and generation (encoding) of OCS-compatible libraries.

As disclosed in Example 7, three identimer classes (HairPin Id₁₋₃) were constructed having scaffold portions comprising a stem duplex portion and a loop portion (referred to herein as “HairPin Identimers”) and were used to construct a set of HairPin Id-based molecular barcodes. The stem duplex portion of each of HairPin Id₁₋₃ comprised first and second oligonucleotides configured to hybridize and form a dsDNA duplex having a free sticky-end available for ligation. Upon forming a duplex, the first and second oligonucleotides comprised first and second restriction endonuclease recognition portions (referred to herein, respectively, as “RE1 Site” and “RE2 Site”). The first oligonucleotide of each of HairPin Id₁₋₃ was configured to have a 5AmMC6 modified nucleotide available for biotinylating the duplex. The second oligonucleotide of each of HairPin Id₁₋₃ was configured to have an internal iAmMC6T modified nucleotide available for fluorescent labeling by a NHS fluorophore. As used in Example 7, each loop portion of HairPin Id₁₋₃ comprised a ssDNA Hp_Dual_NH2 oligonucleotide (SEQ ID NO: 39) configured to form a dsDNA duplex with a stem-loop structure. The nucleotide corresponding to position 1 of SEQ ID NO: 39 was modified by 5′ phosphorylation. Positions 14 and 20 of SEQ ID NO: 39 were Int Amino Modifier C6 dT modified oligonucleotides. Positions 13 to 21 comprise a stem-loop structure. Each loop portion was configured to have one or more iAmMC6T modified nucleotides at positions within the stem-loop structure available for fluorescent labeling by a NHS fluorophore. The scaffold portions of each of HairPin Id₁₋₃ were configured to comprise one or more recognition moieties and amino-modified bases for attachment to a streptavidin bead (via NHS-LC-biotin; commercially available from Sigma-Aldrich, Inc., PO Box 14508, St. Louis, MO 68178, USA; CAS No.: 35013-72-0), and NHS-fluorophores (available commercially from ThermoFisher Scientific, Inc. 168 Third Avenue, Waltham, MA 02451, USA; Cat. No. 65601). All oligonucleotide-streptavidin conjugations were carried out as described previously herein.

The first oligonucleotide of a stem duplex portion of HairPin Id₁ comprised an Ab_stem1_SpeI_XhoI oligonucleotide (SEQ ID NO: 33). The nucleotide corresponding to position 1 of SEQ ID NO: 33 was an Amino Modifier C6 modified nucleotide. Positions 17 to 22 and 31 to 36 of SEQ ID NO: 33 comprised, respectively, a SpeI recognition moiety and a XhoI recognition moiety. Positions 41 to 44 of SEQ ID NO: 33 comprised a sticky-end configured to anneal to the sticky end of SEQ ID NO: 39. The second oligonucleotide of a stem duplex portion of HairPin Id₁ comprised an Ab-stem1 comp oligonucleotide (SEQ ID NO: 34). The nucleotide corresponding to position 1 of SEQ ID NO: 34 was modified by 5′ phosphorylation. Position 14 of SEQ ID NO: 34 was an Int Amino Modifier C6 dT modified nucleotide. Positions 5 to 10 and 18 to 23 of SEQ ID NO: 28 comprised, respectively, a XhoI recognition moiety and a SpeI recognition moiety.

The first oligonucleotide of a stem duplex portion of HairPin Id₂ comprised an Ab_stem2_HindIII_SpeI oligonucleotide (SEQ ID NO: 35). The nucleotide corresponding to position 1 of SEQ ID NO: 35 was modified by Amino Modifier C6. Positions 16 to 21 and 30 to 35 of SEQ ID NO: 35 comprised, respectively, a HindIII recognition moiety and a SpeI recognition moiety. Positions 40 to 43 of SEQ ID NO: 35 comprised a sticky-end configured to anneal to the sticky end of SEQ ID NO: 39. The second oligonucleotide of a stem duplex portion of HairPin Id₂ comprised an Ab-stem2_comp oligonucleotide (SEQ ID NO: 36). The nucleotide corresponding to position 1 of SEQ ID NO: 36 was modified by 5′ phosphorylation. Position 14 of SEQ ID NO: 36 was an Int Amino Modifier C6 dT modified nucleotide. Positions 5 to 10 and 18 to 23 of SEQ ID NO: 36 comprised, respectively, a HindIII recognition moiety and a SpeI recognition moiety.

The first oligonucleotide of a stem duplex portion of HairPin Id₃ comprised an Ab_stem3_EcoRI_HindIII oligonucleotide (SEQ ID NO: 37). The nucleotide corresponding to position 1 of SEQ ID NO: 37 was modified by Amino Modifier C6. Positions 16 to 21 and 30 to 35 of SEQ ID NO: 37 comprised, respectively, a EcoRI recognition moiety and a HindIII recognition moiety. Positions 40 to 43 of SEQ ID NO: 33 comprised a sticky-end configured to anneal to the sticky end of SEQ ID NO: 39. The second oligonucleotide of a stem duplex portion of HairPin Id₃ comprised an Ab-stem3 comp oligonucleotide (SEQ ID NO: 38). The nucleotide corresponding to position 1 of SEQ ID NO: 38 was modified by 5′ phosphorylation. Position 14 of SEQ ID NO: 34 was an Int Amino Modifier C6 dT modified nucleotide. Positions 5 to 10 and 18 to 23 of SEQ ID NO: 28 comprised, respectively, a HindIII recognition moiety and a EcoRI recognition moiety.

A set of Ab_stem1_SpeI_XhoI oligonucleotide, a set of Ab-stem1_SpeI_XhoI oligonucleotides, a set of Ab_stem2_HindIII_SpeI, a set of Ab-stem2_comp oligonucleotide, a set of Ab_stem3_EcoRI_HindIII oligonucleotide, and a set of Ab_stem3_comp oligonucleotides (collectively referred to herein as “stem oligonucleotides”) were synthesized in preparation for duplex formation and conjugation to a set of streptavidin beads.

As used in Example 7, all oligonucleotides were ordered from IDT as either purified oligos (HPLC), or as standard desalted oligos. Standard desalted oligonucleotides containing 5′-amino modifications were first desalted or precipitated to remove any excess free amine carried over from synthesis. The 5′-amino group of the stem oligonucleotides (200 μM) was modified with an appropriate chemistry for attachment to MyOne T1 streptavidin beads by adding NHS-LC-Biotin resuspended in anhydrous DMF (commercially available from Sigma-Aldrich, Inc., PO Box 14508, St. Louis, MO 68178; Cas No.: 68302-57-8) to a final concentration of approximately 2.0 mM, and was allowed to react overnight at room temperature in NHS Conjugation Buffer (NCB: 100 mM NaPO₄ pH 8.5). This reaction was then buffer exchanged two times into water using 7K MWCO Zeba desalting columns (commercially available from ThermoFisher Scientific, Inc., 168 Third Avenue, Waltham, MA 02451, USA; Cat. No. 89891) to remove all excess unreacted biotin.

Labeling of internal-amino modified oligonucleotides (i.e., Ab_stem1_comp, Ab_stem2_comp, Ab_stem3_comp, HP_Dual_NH2) with various NHS-fluorophores was performed as described previously herein (1 mM NHS-fluorophore reagent was reacted with 200 μM oligo in NHS conjugation buffer overnight at room temperature in the dark to prevent fluorophore photobleaching). These reactions were quenched the following day by diluting 10-fold with 2× Hybridization buffer (20 mM Tris-HCl pH 7.5/1M NaCl/1 mM EDTA) to a concentration of 20 μM.

As shown in FIG. 24A, Ab_stem1_SpeI_XhoI was biotinylated, Ab_stem1_comp was labeled with NHS-AF-647, and the two oligonucleotides were annealed at a 1:1.2 molar ratio (respectively; 10 μM biotinylated oligonucleotide: 12 μM labeled oligonucleotide) in 1× hybridization buffer by heating for 5 minutes at 90° C., and slowly cooling to RT over about 30 minutes in a heat block. This annealed, biotinylated and 647-labeled oligonucleotide duplex was ligated in solution to the HP_Dual_NH2 oligonucleotide, which was pre-labeled with a single fluorophore type (NHS-AF750 shown in the example; Thermo) as described above. The in-solution ligation was carried out by mixing the annealed duplex at 200 nM in 1× T4 DNA Ligase buffer (NEB) with AF750-labeled HP_Dual_NH2 oligonucleotide at a final concentration of 400 nM, with 5 μl T4 PNK (NEB) and 5 μl of T4 DNA ligase (NEB) in a 2500 ligation reaction for 30 minutes at 37° C. Ligations were captured directly onto MyOne T1 streptavidin beads pre-washed as described above (2 washes with 2× Hybridization buffer) prior to binding with biotinylated oligonucleotides in the ligation reaction. Binding was initiated by mixing 250 μl of washed beads at 0.2 mg/ml in 2× Hybridization buffer with the 2500 ligation reaction, and was allowed to proceed at 37° C. for 30 minutes. Beads were then washed 3× with 200 μl 2× Hybridization buffer supplemented with 0.01% Tween-20, 2× with 200 μl 1× Hybridization buffer, resuspended in 20 μl of 1× Hybridization buffer, sonicated for 3 minutes, and loaded into a flow cell containing a biotin-modified surface for immobilization and subsequent cleavage experiments.

### Example 9 Encoding and Decoding Molecular Barcodes Produced by ssDNA Hybridization (Hyb) Identimers

As disclosed in Example 9 a set of three Hyb Id-based molecular barcodes were formed and encoded from three identimer classes (Hyb Id₁₋₃), in which each identimer class comprised scaffold portions having a first hybridizing oligonucleotide and a second hybridizing oligonucleotide, the hybridizing nucleotides configured to hybridize to each other and form a biotinylated, fluorescently labeled, dsDNA duplex having one or more orthogonal cleavage sites (referred to collectively as “Hybridization Identimers” or “Hyb Id”) that were used to construct a set of Hyb Id-based molecular barcodes. The scaffold portions of Hyb Id₁₋₃ comprised, respectively, a XhoI recognition moiety, a SpeI recognition moiety, and a NotI recognition moiety.

The scaffold portion of Hyb Id₁ comprised a Hyb_5NH2_XhoI bead first hybridizing oligonucleotide (SEQ ID NO: 40) (also referred to herein as “Hyb_5NH2_XhoI bead oligonucleotide”) and a Hyb_5NH2_XhoI_comp second hybridizing oligonucleotide (SEQ ID NO: 41) (also referred to herein as “Hyb_5NH2_XhoI_comp oligonucleotide”). The nucleotide corresponding to position 1 of SEQ ID NO: 40 was Amino Modifier C6 modified. Positions 22 to 27 of SEQ ID NO: 40 comprised a XhoI recognition moiety (also referred to in Example 9 as “RE3 site”). The nucleotide corresponding to position 1 of SEQ ID NO: 41 was Amino Modifier C6 modified. Positions 8 to 13 of SEQ ID NO: 41 comprised a XhoI recognition moiety.

The scaffold portion of Hyb Id₂ comprised a Hyb_5NH2_SpeI bead first hybridizing oligonucleotide (SEQ ID NO: 42) (also referred as “Hyb_SpeI_XhoI bead oligonucleotide”) and a Hyb_5NH2_SpeI_comp second hybridizing oligonucleotide (SEQ ID NO: 43) (also referred to herein as “Hyb_5NH2_SpeI_comp oligonucleotide”). The nucleotide corresponding to position 1 of SEQ ID NO: 42 was Amino Modifier C6 modified. Positions 21 to 26 of SEQ ID NO: 42 comprised a SpeI recognition moiety (also referred to in Example 9 as “RE2 site”). The nucleotide corresponding to position 1 of SEQ ID NO: 43 was Amino Modifier C6 modified.

The scaffold portion of Hyb Id₃ comprised a Hyb_5NH2_NotI bead first hybridizing oligonucleotide (SEQ ID NO: 44) (also referred to herein as “Hyb_5NH2_XhoI bead oligonucleotide”) and a Hyb_5NH2_XhoI_comp second hybridizing oligonucleotide (SEQ ID NO: 45) (also referred to herein as “Hyb_5NH2_XhoI_comp oligonucleotide”). The nucleotide corresponding to position 1 of SEQ ID NO: 44 was Amino Modifier C6 modified. Positions 22 to 27 of SEQ ID NO: 44 comprised a NotI recognition moiety (also referred to in Example 9 as “RE3 site”). The nucleotide corresponding to position 1 of SEQ ID NO: 41 was Amino Modifier C6 modified.

Amine-reactive ATTO-550 (NHS-ATTO-550) was combined with a set of Hyb_5NH2_XhoI_comp oligonucleotides to label them at their 5′ free amines (i.e., the nucleotide corresponding to position 1 of SEQ ID NO: 41). Amine-reactive AF-647 (NHS-AF-647) was combined with a set of Hyb_5NH2_SpeI_comp oligonucleotides to label them at their 5′ free amines (i.e., the nucleotide corresponding to position 1 of SEQ ID NO: 43). Amine-reactive ATTO-488 (NHS-ATTO-488) was combined with a set of Hyb_5NH2_NotI_comp oligonucleotides to label them at their 5′ free amines (i.e., the nucleotide corresponding to position 1 of SEQ ID NO: 45).

The labeled sets of: Hyb_5NH2_XhoI_comp oligonucleotides, Hyb_5NH2_SpeI_comp oligonucleotides, and Hyb_5NH2_NotI_comp oligonucleotides (collectively referred to in Example 9 as “comp oligonucleotides”) were biotinylated as described already herein, and all three were added to MyOne T1 streptavidin beads for coating. The comp oligonucleotides were labeled with various NHS-fluorophore reagents as described above. For combinatorial encoding of beads coated with biotinylated oligonucleotides, one labeled hybridizing oligonucleotide was introduced at a time during splitting and pooling cycles. These labeled hybridizing oligonucleotides were introduced at a concentration of 200 nM during each encoding cycle, in 0.5× Hybridization buffer. Labeled strands were incubated with beads for 30 minutes at 37° C. with occasional tapping of tubes to promote capture. In between each cycle of encoding, beads were washed 3× with 200 μl 2× Hybridization buffer supplemented with 0.01% Tween-20, 2× with 200 μl 1×

Hybridization buffer, resuspended in 20 μl of 1× Hybridization buffer, and sonicated for 3 minutes. After three rounds of encoding identimers by hybridization were complete, beads were washed 3× with 200 μl 2× Hybridization buffer supplemented with 0.01% Tween-20, 2× with 200 μl 1× Hybridization buffer, resuspended in 20 μl of 1× Hybridization buffer, sonicated for 3 minutes, and loaded into a flow cell containing a biotin-modified surface for immobilization and subsequent cleavage experiments.

Amine-reactive ATTO-550 (NHS-ATTO-550) was combined with a set of Hyb_5NH2_XhoI_comp oligonucleotides to label them at their 5′ free amines (i.e., the nucleotide corresponding to position 1 of SEQ ID NO: 41). Amine-reactive AF-647 (NHS-AF-647) was combined with a set of Hyb_5NH2_SpeI_comp oligonucleotides to label them at their 5′ free amines (i.e., the nucleotide corresponding to position 1 of SEQ ID NO: 43). Amine-reactive ATTO-488 (NHS-ATTO-488) was combined with a set of Hyb_5NH2_NotI_comp oligonucleotides to label them at their 5′ free amines (i.e., the nucleotide corresponding to position 1 of SEQ ID NO: 45). End of Example 9.

### Imaging System Dynamic Range

In some embodiments, methods for imaging may be limited by the dynamic range of the imaging system (see Weissleder et al., IEEE J. Sel. Top Quantum Electron; January-February; 25(1):6801507 (2019)). For example, when constructing libraries as described herein, some streptavidin beads will have multiple copies of the same fluorophore, and any streptavidin beads found to saturate signal in any of the fluorescence channels may be difficult to resolve. Thus, in some embodiments, use of high dynamic range imaging will improve upon the library diversity that one could construct using the compositions and methods disclosed herein. For example, three images are acquired for each experimental data point: one taken at low exposure, one taken at mid-exposure, and one taken at a higher exposure. Those three images are mathematically stitched back together to create one continuous “image” with very high dynamic range. In this way, streptavidin beads containing very few copies of a fluorophore can be imaged in the same field of view (experiment) as streptavidin beads that contain many copies of that fluorophore. Streptavidin beads with very few copies of a fluorophore need a higher exposure to get their values up into a range where they can be accurately quantified, and beads with many copies of a fluorophore need a lower exposure to get their values down below saturation, and into a range where they can be accurately quantified. Thus, in some embodiments, use of high dynamic range imaging allows for streptavidin beads of greater deviation into the same experiment to increase the diversity of libraries constructed with the compositions and methods disclosed herein.

It will be apparent to those having skill in the art that many changes may be made to the details of the above-described embodiments without departing from the underlying principles of the disclosure.

1. A molecular barcode comprising a set of one or more identimers, each identimer in the set of identimers comprising a set of one or more detectable labels operatively connected to a scaffold portion, the scaffold portion comprising:
a recognition moiety that is orthogonal to the recognition moiety of at least one identimer in the set of identimers; and
a cleavage site operatively connected to the set of one or more detectable labels, concatenated to each other in a three-dimensional arrangement to encode and form the molecular barcode.

2. The molecular barcode of claim 1, whereby applying a cleavage agent orthogonally reactive to the recognition moiety cleaves the molecular barcode at the cleavage site of at least one identimer to dissociate the one or more detectable labels from three-dimensional arrangement according to orthogonality of the identimers and thereby induce a detectable signal response to decode the molecular barcode.

3. The molecular barcode of claim 1 or claim 2, wherein the scaffold portion comprises polypeptides, amino acids, cyclic peptides, nucleic acids, or a combination thereof.

4. The molecular barcode of any one of claims 1-3, wherein the scaffold portion comprises double-stranded DNA (dsDNA), the recognition moiety comprises a nuclease recognition moiety, and the one or more detectable labels comprise a fluorophore.

5. The molecular barcode of any one of claims 1-4, wherein the scaffold portion comprises double-stranded DNA, the recognition moiety comprises a protease recognition moiety, and the one or more detectable labels comprise a fluorophore.

6. The molecular barcode of any one of claims 1-4, wherein the scaffold portion comprises double-stranded DNA, the recognition moiety comprises a chemical cleavage recognition moiety, and the one or more detectable labels comprise a fluorophore.

7. The molecular barcode of any one of claims 1-4, wherein the scaffold portion comprises amino acids, the recognition moiety comprises a protease recognition moiety, and the one or more detectable labels comprise a fluorophore.

8. The molecular barcode of any one of claims 1-4, wherein the scaffold portion comprises amino acids, the recognition moiety comprises a chemical cleavage recognition moiety, and the one or more detectable labels comprise a fluorophore.

9. The molecular barcode of any one of claims 1-8, wherein the recognition moiety comprises the cleavage site.

10. The molecular barcode of any one of claims 1-9, further comprising an identimer linker between each identimer in the set of identimers.

11. The molecular barcode of claim 10, wherein the identimer linker is selected from a group of click chemistry moieties, native chemical ligation linkers, and enzyme-mediated ligation linkers.

12. The molecular barcode of any one of claims 1-11, wherein the recognition moiety of at least one identimer in the set of identimers comprises a chemical linker, peptide, or nucleic acid.

13. The molecular barcode of any one of claims 1-12, wherein at least one or more of the detectable labels in the set of identimers comprises a fluorophore.

14. The molecular barcode of any one of claims 1-13, wherein each recognition moiety in the set of identimers comprises at least one moiety selected from a protease recognition moiety, an endonuclease recognition moiety, an epitope recognizable by an affinity reagent, a nucleic acid probe recognition moiety, a modified peptide side chain, and an unnatural peptide side chain.

15. The molecular barcode of any one of claims 1-14, wherein the molecular barcode is attached to a bead.

16. The molecular barcode of claim 15, wherein the bead is further attached to a test agent.

17. The molecular barcode of claim 15, wherein the bead is further attached to chemical building block.

18. The molecular barcode of any one of claims 1-17, further comprising a material having mutual information with the three-dimensional arrangement of the molecular barcode.

19. A method of classifying material comprising:
introducing into a milieu, a set of one or more molecular barcodes of any of claims 1 through 18, wherein at least one molecular barcode in the set has mutual information with a material;
selectively applying orthogonally reactive cleaving agents to the set of molecular barcodes to cleave the molecular barcodes at the cleavage sites that are reactive to the cleaving agents to dissociate the detectable labels of identimers from the three-dimensional arrangements according to the orthogonality of the identimers and thereby induce a detectable signal response to decode the molecular barcodes.

20. A method of screening, comprising:
introducing a plurality of test constructs into a milieu, wherein each test construct comprises a test agent operatively coupled to a molecular barcode of any one of claims 1-18, wherein the three-dimensional arrangement of the molecular barcode has mutual information with the test agent;
screening the plurality of test constructs against a set of one or more targets;
detecting an activity of one or more of the test constructs;
selectively applying orthogonally reactive cleaving agents to the plurality of test constructs to cleave coupled molecular barcodes at the cleavage sites that are reactive to the cleaving agents to dissociate the detectable labels of identimers from the three-dimensional arrangements according to the orthogonality of the identimers and thereby induce a detectable signal response to decode the couple barcodes and thereby identify test agents with activity against the set of targets.

21. The method of claim 19 or claim 20, wherein the detectable signal response is induced without enrichment.

22. The method of claim 20 or claim 21, wherein selectively applying orthogonally reactive cleavage agents to the plurality of test constructs comprises applying the cleavage reagents sequentially.

23. The method of claim 22, wherein applying the cleavage reagents sequentially comprises applying a single cleaving agent iteratively or with two or more cleaving agents sequentially.

24. The method of any one of claims 19-23, wherein the detectable signal response comprises detecting the presence or absence of one more or more dissociated detectable labels or the modulation of a signal frequency or signal amplitude.

25. A method for generating a molecular barcode, comprising:
providing a set of one or more identimers, each identimer in the set of identimers comprising: a set of one or more detectable labels operatively connected to a scaffold portion, the scaffold portion comprising a recognition moiety that is orthogonal to the recognition moiety of at least one identimer in the set of identimers and a cleavage site operatively connected to a set of one or more detectable labels;
selecting first and second identimers from the set of identimers and concatenating the first identimer to the second identimer in a three-dimensional arrangement to encode and form a molecular barcode;
(optionally) selecting a identimer from the set of identimers and concatenating it to the molecular barcode to modify the three dimensional arrangement and further encode and form the molecular barcode;
(optionally) repeating one to nth times the step of selecting a identimer from the set of identimers and concatenating it to the molecular barcode.

26. The method of claim 25, further comprising operatively attaching the first identimer to a material.

27. The method of claim 26, wherein the material is a surface.

28. The method of claim 27, wherein the surface comprises an outer or inner surface of a microparticle or bead.

29. The method of claim 27 or claim 28, comprising operatively attaching the first identimer to the surface by a high-affinity binding protein.

30. The method of claim 25, further comprising attaching the first identimer to a three-armed linker.

31. The method of claim 25, wherein providing a set of one or more identimers further comprises configuring each identimer in the set of identimers to concatenate to a preceding identimer and to facilitate concatenation of the identimers in a linear three-dimensional arrangement and thereby form and encode the molecular barcode sequentially.

32. The method of claim 31, wherein configuring the set of identimers to bind specifically to a preceding identimer comprises concatenating by ligation to, extension from, or synthesizing onto, the preceding identimer.

33. The method of any one of claims 25-33, further comprising:
providing a set of one or more chemical building blocks, each chemical building block in the set of chemical building blocks being configured to concatenate to a preceding chemical building block;
selecting a first chemical building block from the set of chemical building blocks and concatenating it to the first identimer;
(optionally) selecting a chemical building block from the set of chemical building blocks configured to concatenate to the preceding chemical building block and concatenating it to the preceding chemical building block and thereby form a series of chemical building blocks; and
(optionally) repeating one to Nth times the step of selecting a chemical building block from the set of chemical building blocks configured to concatenate to the preceding chemical building block and concatenating it to the preceding chemical building block.

34. The method of claim 33, wherein concatenating the series of chemical building blocks comprises the use of non-nucleic acid compatible reactions.

35. A system for encoding and decoding a molecular barcode comprising:
a barcode encoding system configured to introduce into a milieu a set of one or more encoded molecular barcodes of any of claims 1 through 8, wherein at least one molecular barcode in the set has mutual information with a material;
a cleavage system configured to selectively apply orthogonally reactive cleaving agents to milieu to cleave the set of molecular barcodes at the cleavage sites of identimers having recognition moieties that are reactive to the cleaving agents to dissociate the detectable labels from the three-dimensional arrangement of molecular barcodes in the set of molecular barcodes according to the orthogonality the identimers and thereby induce a detectable signal response from one to decode the set of molecular barcodes;
(optionally) an illumination system configured to selectively convey light to the set of molecular barcodes to induce the detectable signal response;
a detection system for detecting the detectable signal response from the set of molecular barcodes; and
a processor coupled to the illumination system and the optical detection system and configured to facilitate decoding the set of molecular barcodes.


SEQUENCE LISTING (45 sequences)

SEQ ID NO: 1 - protein, 21 aa, Artificial Sequence
  Peptide sequence containing TUMV cleavage site.
  SITE (17)..(21): An azide bearing residue is installed internally near the C-terminus.
KGGSGGGSACVYHQSGGGGSC

SEQ ID NO: 2 - protein, 22 aa, Artificial Sequence
  Peptide sequence containing a SuMMV cleavage site.
  SITE (17)..(21): An azide bearing residue is installed internally near the C-terminus.
KGGSGGGSEEIHLQSGGGGGSC

SEQ ID NO: 3 - protein, 21 aa, Artificial Sequence
  Peptide sequence containing a TVMV cleavage site.
  SITE (17)..(21): An azide bearing residue is installed internally near the C-terminus.
KGGSGGGSETVRFQGGGGGSC

SEQ ID NO: 4 - protein, 21 aa, Artificial Sequence
  Peptide sequence containing TEV cleavage site.
  SITE (17)..(21): An azide bearing residue is installed internally near the C-terminus.
KGGSGGGSENLYFQSGGGGSC

SEQ ID NO: 5 - DNA, 24 bp, Artificial Sequence
  Nucleotide sequence containing a HindIII cleavage site. The nucleotide corresponding to position 19 is modified by Int Amino Modifier C6.
atttatttaagcttattattattt

SEQ ID NO: 6 - DNA, 25 bp, Artificial Sequence
  Nucleotide sequence containing a SpeI cleavage site. The nucleotide corresponding to position 20 is modified by Int Amino Modifier C6.
atttatttaactagtattattattt

SEQ ID NO: 7 - DNA, 25 bp, Artificial Sequence
  Nucleotide sequence containing an XhoI cleavage site. The nucleotide corresponding to position 20 is modified by Int Amino Modifier C6.
atttatttactcgagattattattt

SEQ ID NO: 8 - DNA, 27 bp, Artificial Sequence
  Nucleotide sequence containing a NotI cleavage site. The nucleotide corresponding to position 22 is modified by Int Amino Modifier C6.
atttatttagcggccgcattattattt

SEQ ID NO: 9 - protein, 22 aa, Artificial Sequence
  Peptide sequence containing a TUMV cleavage site.
KGGSGGGSACVYHQSGGSGGSC

SEQ ID NO: 10 - protein, 22 aa, Artificial Sequence
  Peptide sequence containing a SuMMV cleavage site.
KGGSGGGSEEIHLQSGGSGGSC

SEQ ID NO: 11 - protein, 22 aa, Artificial Sequence
  Peptide sequence containing a TVMV cleavage site.
KGGSGGGSETVRFQGGGSGGSC

SEQ ID NO: 12 - protein, 22 aa, Artificial Sequence
  Peptide sequence containing a TEV cleavage site.
KGGSGGGSENLYFQSGGSGGSC

SEQ ID NO: 13 - DNA, 25 bp, Artificial Sequence
  Nucleotide sequence containing a HindIII cleavage site. The nucleotide corresponding to position 1 is modified by a Amino Modifier C6.
catttatttaagcttattattattt

SEQ ID NO: 14 - DNA, 16 bp, Artificial Sequence
  Nucleotide sequence containing a SpeI cleavage site. The nucleotide corresponding to position 1 is modified by a Amino Modifier C6.
actagtattattattt

SEQ ID NO: 15 - DNA, 25 bp, Artificial Sequence
  Nucleotide sequence containing a XhoI cleavage site. The nucleotide corresponding to position 1 is modified by a Amino Modifier C6.
atttatttactcgagattattattt

SEQ ID NO: 16 - DNA, 27 bp, Artificial Sequence
  Nucleotide sequence containing a NotI cleavage site. The nucleotide corresponding to position 1 is modified by a Amino Modifier C6.
atttatttagcggccgcattattattt

SEQ ID NO: 17 - DNA, 31 bp, Artificial Sequence
  Biotinylated with 5BiotinTeg at position 1.
attattttattagctagtacgctacgacgtc

SEQ ID NO: 18 - DNA, 25 bp, Artificial Sequence
  Positions 1 to 5 comprise a sticky end configured to anneal to the sticky end of SEQ ID NO 19. The nucleotides corresponding to positions 1 and 25 are modified, respectively, by 5′ phosphoylation and a 3′ Amino Modifier C6 dT modified nucleotide.
tgcaggacgtcgtagcgtactagct

SEQ ID NO: 19 - DNA, 28 bp, Artificial Sequence
  Positions 1 to 5 comprise a sticky end configured to anneal to the sticky end of SEQ ID NO 18. Positions 13-18 comprise a HindIII cleavage site. Positions 1 is modified by 5Phos.
ctgcacgactgcaagcttgatactagca

SEQ ID NO: 20 - DNA, 28 bp, Artificial Sequence
  Positions 1 to 5 comprise a sticky end configured to anneal to the sticky end of SEQ ID NO 21. Positions 16-21 comprise a HindIII cleavage site. Positions 1 and 9 are modified by, respectively, 5Phos and iAmMC6T.
acacttgctagtatcaagcttgcagtcg

SEQ ID NO: 21 - DNA, 28 bp, Artificial Sequence
  The nucleotides corresponding to positions 1 to 5 comprise a sticky end configured to anneal to the sticky end of SEQ ID NO 20. Positions 13 to 18 comprise a SpeI recognition moiety and cleavage site. Position 1 is modified by 5Phos.
agtgtcgactgcactagtgatactagca

SEQ ID NO: 22 - DNA, 28 bp, Artificial Sequence
  Positions 1 to 5 comprise a sticky end configured to anneal to the sticky end of SEQ ID NO 23. Positions 16 to 21 comprise a SpeI recognition moiety and cleavage site. Positions 1 and 9 are modified by, respectively, 5Phos and iAmMC6T.
tatgctgctagtatcactagtgcagtcg

SEQ ID NO: 23 - DNA, 28 bp, Artificial Sequence
  Positions 1 to 5 comprise a sticky end configured to anneal to the sticky end of SEQ ID NO 23. Positions 13 to 18 comprise a Xho1 recognition moiety and cleavage site. Position 1 is modified by 5Phos.
gcatacgactgcctcgaggatactagca

SEQ ID NO: 24 - DNA, 28 bp, Artificial Sequence
  Positions 1 to 5 comprise a sticky end configured to anneal to the sticky end of SEQ ID NO 25. Positions 16 to 21 comprise an XhoI recognition moiety and cleavage site. Positions 1 and 9 are modified by, respectively, 5Phos and iAmMC6T.
atggatgctagtatcctcgaggcagtcg

SEQ ID NO: 25 - DNA, 28 bp, Artificial Sequence
  Positions 1 to 5 comprise a sticky end configured to anneal to the sticky end of SEQ ID NO 24. Positions 11 to 18 comprise a NotI recognition moiety and cleavage site. Position 1 is modified by 5Phos.
tccatcgactgcggccgcgatactagca

SEQ ID NO: 26 - DNA, 23 bp, Artificial Sequence
  Position 1 comprises a 5AmMc6 modified nucleotide. Positions 11 to 18 comprise a NotI recognition moiety and cleavage site.
tgctagtatcgcggccgcagtcg

SEQ ID NO: 27 - DNA, 37 bp, Artificial Sequence
  Position 1 is 5Phos modified. Postions 14 and 20 are iAmMC6T modified nucleotides. Positions 34 to 37 comprise a sticky-end configured to anneal to the sticky end of SEQ ID NO 28.
  stem_loop (13)..(22)
cgagctcgtcggtttttttttccgacgagctcgaaat

SEQ ID NO: 28 - DNA, 37 bp, Artificial Sequence
  Position 1 is 5Phos modified. Postion 17 is a iAmMC6T modified nucleotide. Positions 34 to 37 comprise a sticky-end configured to anneal to the sticky end of SEQ ID NO 27.
  stem_loop (13)..(21)
cgcgctcgtcggtttttttttccgacgagcgcgattt

SEQ ID NO: 29 - DNA, 37 bp, Artificial Sequence
  Position 1 is 5Phos modified. Postions 14 and 20 are iAmMC6T modified nucleotides. Positions 2 to 7 and 27 to 32 are SpeI recognition moieties. Positions 34 to 37 comprise a sticky-end configured to anneal to the sticky end of SEQ ID NO 30.
  stem_loop (13)..(21)
cactagtgtcggtttttttttccgacactagtgcaga

SEQ ID NO: 30 - DNA, 37 bp, Artificial Sequence
  Position 1 is 5Phos modified. Postion 17 is a iAmMC6T modified nucleotide. Positions 34 to 37 comprise a sticky-end configured to anneal to the sticky end of SEQ ID NO 29.
  stem_loop (13)..(21)
cgcgctcgtcggtttttttttccgacgagcgcgtctg

SEQ ID NO: 31 - DNA, 41 bp, Artificial Sequence
  Position 1 is 5Phos modified. Postions 16 and 22 are iAmMC6T modified nucleotides. Positions 38 to 41 comprise a sticky-end configured to anneal to the sticky end of SEQ ID NO 32. Positions 2 to 9 and 29 to 36 comprise NotI recognition moieties.
  stem_loop (15)..(23)
cgcggccgcgtcggtttttttttccgacgcggccgcgttac

SEQ ID NO: 32 - DNA, 37 bp, Artificial Sequence
  Position 1 is 5Phos modified. Postion 17 is a iAmMC6T modified nucleotide. Positions 34 to 37 comprise a sticky-end configured to anneal to the sticky end of SEQ ID NO 31.
  stem_loop (13)..(21)
cgcgctcgtcggtttttttttccgacgagcgcggtaa

SEQ ID NO: 33 - DNA, 43 bp, Artificial Sequence
  Position 1 is 5AmMc6 modified. Positions 17 to 22 and 31 to 36 comprise, respectively, a SpeI regonition moiety and a XhoI recognition moiety. Positions 41 to 44 comprise a sticky-end configured to anneal to the stick-end of SEQ ID NO 39.
tttttttatttatttactagtatttatttctcgagatttattt

SEQ ID NO: 34 - DNA, 32 bp, Artificial Sequence
  Position 1 is 5Phos modified. Position 14 is a iAmMC6T modified nucleotide. Positions 5 to 10 and 18 to 23 and comprise, respectively, a XhoI recognition moiety and a SpeI recognition moiety.
aaatctcgagaaataaatactagtaaataaat

SEQ ID NO: 35 - DNA, 43 bp, Artificial Sequence
  Position 1 is 5AmMc6 modified. Positions 17 to 22 and 31 to 36 comprise, respectively, a HindIII regonition moiety and a SpeI recognition moiety. Positions 41 to 44 comprise a sticky-end configured to anneal to the stick-end of SEQ ID NO 39.
tttttttatttatttaagcttatttatttactagtatttattt

SEQ ID NO: 36 - DNA, 32 bp, Artificial Sequence
  Position 1 is 5Phos modified. Position 14 is a iAmMC6T modified nucleotide. Positions 5 to 10 and 18 to 23 and comprise, respectively, a HindIII recognition moiety and a SpeI recognition moiety.
aaatactagtaaataaataagcttaaataaat

SEQ ID NO: 37 - DNA, 43 bp, Artificial Sequence
  Position 1 is 5AmMc6 modified. Positions 16 to 21 and 30 to 35 comprise, respectively, a EcoRI regonition moiety and a HindIII recognition moiety. Positions 40 to 43 comprise a sticky-end configured to anneal to the stick-end of SEQ ID NO 39.
tttttttatttatttgaattcatttatttaagcttatttattt

SEQ ID NO: 38 - DNA, 32 bp, Artificial Sequence
  Position 1 is 5Phos modified. Position 14 is a iAmMC6T modified nucleotide.
aaataagcttaaataaatgaattcaaataaat

SEQ ID NO: 39 - DNA, 37 bp, Artificial Sequence
  Position 1 is 5Phos modified. Positions 14 and 20 are iAmMc6T modified nucleotide. Positions 13 to 21 comprise a stem-loop structure.
  stem_loop (13)..(21)
cgcgctcgtcggtttttttttccgacgagcgcgaaat

SEQ ID NO: 40 - DNA, 34 bp, Artificial Sequence
  Position 1 modified by 5AmMC6. Positions 22 to 27 comprise a XhoI recognition moiety.
tttttttatttattttccgacgagctcagcattt

SEQ ID NO: 41 - DNA, 20 bp, Artificial Sequence
  Position 1 modified by 5AmMC6. Positions 8 to 13 comprise a SpeI recognition moiety.
aaatgctgagctcgtcggaa

SEQ ID NO: 42 - DNA, 33 bp, Artificial Sequence
  Position 1 modified by 5AmMC6. Positions 21 to 26 comprise a SpeI recognition moiety.
tttttttatttattttagatactagtgatactt

SEQ ID NO: 43 - DNA, 20 bp, Artificial Sequence
  Position 1 modified by 5AmMC6.
aagtatcactagtatctaaa

SEQ ID NO: 44 - DNA, 35 bp, Artificial Sequence
  Position 1 modified by 5AmMC6. Positions 21 to 26 comprise a NotI recognition
tttttttatttattttcatagcggccgcgtatctt

SEQ ID NO: 45 - DNA, 22 bp, Artificial Sequence
  Position 1 modified by 5AmMC6.
aagatacgcggccgctatgaaa